(19) United States
(12) Patent Application Publication
(10) Pub. No.: US 2022/0323464 A1
(43) Pub. Date: Oct. 13, 2022

(54) COMPOSITION, FORMULATIONS AND METHODS OF MAKING AND USING BOTANICALS AND NATURAL COMPOUNDS FOR THE PROMOTION OF HEALTHY BRAIN AGING

(71) Applicant: The Brain Health Project, LLC, Boca Raton, FL (US)

(72) Inventor: Morris Notelovitz, Boca Raton, FL (US)

(21) Appl. No.: 17/531,404

(22) Filed: Nov. 19, 2021

Related U.S. Application Data
(63) Continuation of application No. 17/206,374, filed on Mar. 19, 2021.
Application No. 17/206,374 is a continuation of application No. 16/992,456, filed on Aug. 13, 2020, now abandoned.
Application No. 16/992,456 is a continuation of application No. 16/717,283, filed on Dec. 17, 2019, now abandoned.
Application No. 16/717,283 is a continuation of application No. 16/403,449, filed on May 3, 2019, now abandoned.
Application No. 16/403,449 is a continuation of application No. 16/129,786, filed on Sep. 12, 2018, now abandoned.
Application No. 16/129,786 is a continuation of application No. 15/872,504, filed on Jan. 16, 2018, now abandoned.
Application No. 15/872,504 is a continuation of application No. 15/475,626, filed on Mar. 31, 2017, now abandoned.
Application No. 15/475,626 is a continuation of application No. 15/241,883, filed on Aug. 19, 2016, now abandoned.
Application No. 15/241,883 is a continuation of application No. 14/785,565, filed on Oct. 19, 2015, now abandoned.
(60) Provisional application No. 61/812,956, filed on Apr. 17, 2013.

(51) Int. Cl. A61K 31/593; A23L 33/10; A61K 31/4748; A61K 31/353; A61K 31/592; A61K 31/566; G01N 33/68

(52) U.S. Cl. CPC A61K 31/593; A23L 33/10; A61K 31/4748; A61K 31/353; A61K 31/592; A61K 31/566; G01N 33/6896; A23V 2002/00; G01N 2800/28; G01N 2800/52; A61K 2300/00

(57) ABSTRACT

The present disclosure provides compositions and formulations comprising botanicals and natural compounds for the promotion of healthy brain aging in adults, especially adult women, and for prevention of age associated neurodegenerative changes resulting in cognitive, memory and executive dysfunction including modulation of the age related predisposition to mild cognitive impairment, Alzheimer's disease, hormonal and other dementia related conditions. The present disclosure also provides methods of using the compositions and formulations in treating and preventing neurodegenerative changes resulting in cognitive, memory and executive dysfunction.

### RELATED APPLICATION

This application claims the benefit of priority to U.S. Application 61/812,956, filed Apr. 17, 2013, hereby expressly incorporated by reference in its entirety.

### BACKGROUND

The physiologic aging of the human brain is associated with cellular, molecular and functional changes that frequently results in neurocognitive frailty: reduced cognition, memory, mood and executive function. Healthy brain aging is subject to the neurobiology of identifiable genetic factors and the influence of modifiable neuronal and glial cell modulators. The latter will determine neuronal survival; the synthesis and function of neurotrophins and their effect on neurogenesis; synaptic activity and control of its neurotransmitter long term potential; cellular dysfunction associated with inflammatory signals and oxidative stress; metabolic abnormalities linked to insulin resistance and its disruption of the vital pathways regulating brain energy requirements and neuronal survival, and the integrity of the blood brain barrier (Glorioso and Sibille 2011; Uranga et al 2010; Park and Reuer Lorenz 2009; de la Monte 2012; Zlokovic 2008).

When women with underlying neurodegenerative disease are excluded, normal brain aging is not characterized by neuronal death. The cognitive decline is the result of neuronal dendritic arbor shrinkage and a reduction in synaptic density and plasticity (Glorioso and Sibille 2011). The degree to which this occurs determines the continuation of normal cognition (healthy aging) versus cognitive dysfunction and resulting functional impairment (unhealthy aging). The latter also has the potential to stimulate and promote underlying neurological disease related genes into a pro-disease direction. Examples include subjects with a genetic variant of the APOE e4 mutation (Mayeux 2010) and women with insulin resistant type two diabetes (de la Monte 2012).

Various factors are involved in promoting and maintaining brain health. Both environmental and genetic factors may play a role in healthy brain aging. As examples, neurogenesis, oxidative stress, apoptosis, and healthy blood brain barrier are involved in promoting and maintaining brain health. Different growth factors, such as neurotrophins and transforming growth factors, and neurotransmitters may be involved in neurogenesis and neuroprotection of the brain. Other factors may be involved in maintaining the molecular pathways that govern the function of the brain as a person ages.

### SUMMARY

The present application provides compositions comprising Huperzine A or a derivative or analog thereof; one or more estrogens and/or phytoestrogens; and a vitamin D. The estrogen can be selected from the group consisting of estradiol, conjugated equine estrogens (CEE), any active estrogenic ingredients of CEE, estrone, estriol, esterified estrogens, any derivative, analog, or metabolite of the mammalian estrogen and combinations thereof. The estradiol can be 17-beta estradiol, estradiol valerate, ethinyl estradiol, any other estradiol derivative, analog, or metabolite thereof, and combinations thereof. The phytoestrogen can be a soy phytoestrogen. The phytoestrogen can be selected from the group consisting of an isoflavone, a coumestan, a lignan, synthetic analogs and derivatives thereof, and combinations thereof. Examples of Vitamin D, include but are not limited, to calcitriol, doxercalciferol, paricalcitol, cholecalciferol (vitamin D3), ergocalciferol (vitamin D2), analogs and derivatives thereof, Vitamin D receptor agonists and modulators, and combinations thereof. The components of the composition can be natural or endogenous molecules, synthetic molecules, and combinations thereof. The natural or endogenous molecule can be from a mammalian source.

The composition can be a pharmaceutical composition or a nutraceutical composition. In some embodiments, the pharmaceutical composition contains one or more synthetic components. In other embodiments, the nutraceutical composition contains one or more natural components. In other embodiments, the composition can include a combination of synthetic and natural components.

In one embodiment, the pharmaceutical composition comprises genistein, vitamin D3, synthetic Huperzine A, and 17-beta estradiol. In another embodiment, the pharmaceutical composition comprises Huperzine A, genistein and vitamin D in the form of synthetic compounds. The amount of genistein can be about 25 mg to about 40 mg or about 30 mg. The amount of vitamin D3 can be about 600 iu, the amount of Huperazine A can be about 100 mcg, and the amount of 17-beta estradiol can be about 0.2 mg to about 0.5 mg or about 0.3 mg. In another embodiment, the composition comprises genistein and 17-beta estradiol in addition to vitamin D and Huperzine A.

In one embodiment, the composition comprises from about 0.01 mg to about 150 mg of Huperzine A or a analog or derivative thereof, from about 0.01 mg to about 1000 mg of at least one phytoestrogen, and from about 200 iu to about 5000 iu of vitamin D, an analog thereof, or a vitamin D receptor agonist and modulator. In another embodiment, the composition comprises about 50 mcg, about 175 mcg, about 275 mcg, or about 375 mcg of Huperzine A. In one embodiment, the analog or derivative can be a synthetic analog or derivative thereof.

In one aspect, the composition comprises Huperzine A, soy isoflavone, and vitamin D. In another aspect, the composition comprises from about 40 mcg to about 400 mcg of Huperzine A, about 110 mg of soy isoflavone, and about 1200 iu of vitamin D.

The composition can include one or more additives. Additives can be selected from the group consisting of coffee, xanthine alkaloids, chlorogenic acid, sweeteners and combinations thereof. Examples of xanthine alkoid include, but are not limited to, caffeine, theobromine, paraxanthine, and combinations thereof. The sweetener can be a low glycemic sweetener, such as sucromalt, tagatose, isomalt, sucralose, acesulfame potassium, analogs and derivatives thereof, and combinations thereof.

In one embodiment, the composition comprises from about 10 mg to about 100 mg of xanthine alkaloid and/or from about 10 g to about 100 g of a sweetener. In another embodiment, the composition comprises Huperzine A, soy isoflavone, vitamin D, caffeine, and sucromalt. In one aspect, the composition comprises from about 40 mcg to about 400 mcg of Huperzine A, about 110 mg of soy isoflavone, about 1200 iu of vitamin D, about 75 mg of caffeine, and about 75 g of sucromalt.

The composition described herein is a pharmaceutical composition and further comprises one or more pharmaceutically acceptable carriers or excipients. In one embodiment, the composition is formulated for immediate release, extended release, or timed release. The composition can be formulated for oral administration, topical administration, transdermal administration, mucosal administration, buccal administration and combinations thereof. The composition can be formulated in the form of a tablet, a capsule, a powder, an emulsion, a suspension, a syrup, a solution, a gel, and a patch.

As described herein, the composition is useful for preventing, inhibiting, retarding, or treating neuronal degeneration in a subject. Accordingly, provided herein are methods of using the claimed composition to prevent, inhibit, retard, or treat neuronal degeneration in a subject, wherein the method comprises administering an effective amount of the disclosed pharmaceutical composition to a subject in need thereof.

Described herein is also a method of preventing, inhibiting, retarding, or treating decline in cognitive function, executive function, and/or memory in a subject, wherein the method comprises administering an effective amount of the disclosed pharmaceutical composition to a subject in need thereof. In one embodiment, the subject is at risk for or is being treated for type II diabetes. In another embodiment, the subject is receiving hormone therapy including selective estrogen modulators. The hormone is estrogen can be synthetic or mammalian. The subject can have hypercholesterolemia or be at risk for developing cardiovascular disease. The subject can also have osteoporosis or osteopenia.

Described herein is a method of treating an increased risk of a cognitive function, executive function, or memory disorder in a subject, wherein the method comprises administering an effective amount of the disclosed pharmaceutical composition to a subject in need thereof.

Described herein is a method of modulating, treating, inhibiting, retarding, or preventing oxidative stress in the central nervous system of a subject, wherein the method comprises administering an effective amount of the disclosed pharmaceutical composition to a subject in need thereof.

Described herein is a method of promoting healthy brain aging of a subject, wherein the method comprises administering an effective amount of the disclosed pharmaceutical composition to a subject in need thereof.

Described herein is a method of promoting neuronal cell dendritic arborization and synaptic long term potential, wherein the method comprises administering an effective amount of the disclosed pharmaceutical composition to neuronal cells.

Described herein is a method of stimulating the production of neurotrophins and neurotransmitters, wherein the method comprises administering an effective amount of the disclosed pharmaceutical composition to neuronal cells. The neurotrophins are selected from the group consisting of brain derived neurotrophic factor, nerve growth factor, and combinations thereof. The neurotransmitters are selected from the group consisting of serotonin, glutamate, acetylcholine, and combinations thereof.

Described herein is a method of inhibiting apoptosis of neuronal cells, wherein the method comprises administering an effective amount of the disclosed pharmaceutical composition to neuronal cells.

Described herein is a method of inducing neurogenesis of cells, wherein the method comprises administering an effective amount of the disclosed pharmaceutical composition to stem cells or progenitor cells. The cells are neural stem cells or neural progenitor cells.

Described herein is a method of inhibiting apoptosis of neuronal cells, wherein the method comprises administering an effective amount of the disclosed pharmaceutical composition to neuronal cells. The method comprises promoting the expression of Bcl-2 and/or inhibiting the expression of P53 or Bax.

Described herein is a method of inhibiting the formation of amyloid plaques, wherein the method comprises administering an effective amount of the disclosed pharmaceutical composition to neuronal cells expressing amyloid precursor protein (APP). The method comprises stimulating cleavage of APP via the alpha secretase pathway and inhibiting beta and gamma secretase pathways.

Described herein is a method of inhibiting the formation of neurofibrillary tangles, wherein the method comprises administering an effective amount of the disclosed pharmaceutical composition to neuronal cells expressing tau protein. The method comprises deacetylating the tau protein.

Described herein is a method of inhibiting activation of microglial cells, wherein the method comprises administering an effective amount of the disclosed pharmaceutical composition to microglial cells. The method further comprises inhibiting secretion of inflammatory cytokines.

Described herein is a method of inducing the expression of sirtuin genes, wherein the method comprises administering an effective amount of the disclosed pharmaceutical composition to neuronal cells or glial cells expressing sirtuin genes. The sirtuin gene is a SIRT1 gene.

Described herein is a method of maintaining the integrity of the blood brain barrier (BBB), wherein the method comprises administering an effective amount of the disclosed pharmaceutical composition to endothelial cells of the BBB.

Described herein is a method of facilitating glucose transport across the BBB, wherein the method comprises administering an effective amount of the disclosed pharmaceutical composition to endothelial cells of the BBB.

Described herein is a method of inhibiting insulin resistance in neuronal cells, wherein the method comprises administering an effective amount of the disclosed pharmaceutical composition to the neuronal cells.

Described herein is a method of inducing insulin sensitivity in neuronal cells, wherein the method comprises administering an effective amount of the disclosed pharmaceutical composition to the neuronal cells.

Described herein is a method of promoting an increase in efflux of beta amyloid from neuronal cells into the blood stream, wherein the method comprises administering an effective amount of the disclosed pharmaceutical composition to neuronal cells.

Described herein is a method of enhancing the bioactivity of vitamin D in neuronal cells, wherein the method comprises sequenced absorption of an effective amount of the disclosed pharmaceutical composition.

The methods described herein comprise administering an effective amount the disclosed pharmaceutical composition to cells in a subject in need thereof or in need of such treatment.

Described herein is a method of preventing, modulating, or treating mild cognitive impairment and/or Alzheimer's disease, wherein the method comprises administering an effective amount of the disclosed pharmaceutical composition to a subject diagnosed with mild cognitive impairment and/or Alzheimer's disease.

Described herein is a method for alleviating the symptoms of mild cognitive impairment and/or Alzheimer's disease, wherein the method comprises administering an effective amount of the disclosed pharmaceutical composition to a subject diagnosed with mild cognitive impairment and/or Alzheimer's disease.

Described herein is a method of preventing, retarding, or substantially inhibiting mild cognitive impairment and/or Alzheimer's disease, wherein the method comprises administering an effective amount of the disclosed pharmaceutical composition to a subject at risk of developing mild cognitive impairment and/or Alzheimer's disease.

Described herein is a method of preventing, retarding, or treating dementia, wherein the method comprises administering an effective amount of the disclosed pharmaceutical composition to a subject in need thereof.

Described herein is a method of promoting an increase in efflux of beta amyloid from neuronal cells into the blood stream, wherein the method comprises administering an effective amount of the disclosed pharmaceutical composition to neuronal cells.

Described herein is a method of individualizing the dosage of the disclosed composition for the promotion of brain health and treatment of cognitive dysfunction and age related dementia including mild cognitive impairment and Alzheimer's Disease, wherein the method comprises administering the disclosed pharmaceutical composition to a subject in need thereof.

Described herein is a method of measuring and/or monitoring the absorption of bioactive levels of the disclosed pharmaceutical composition, wherein the method comprises administering the composition to a subject in need thereof and measuring and/or monitoring the absorption of bioactive levels of the components of the composition to determine whether an optimal level has been reached. An optimal level may be a range of concentration or level. An optimal level indicates that the subject is receiving an effective amount of the components for promoting brain health or treatment or prevention of diseases or conditions associated with mild cognitive impairment (MCI) or Alzheimer's disease (AD). An optimal level can also indicate that the subject is receiving an effective amount of components for treatment of an adjuctive disease or condition. The adunctive disease can be selected from the group consisting of hypercholesteremia, metabolic syndrome, type II diabetes, obesity, osteopenia, osteoporosis, hypertension, post menopausal hormone replacement therapy and combinations thereof.

Described herein is a method of measuring and monitoring the bioactive brain health protective efficacy of the disclosed pharmaceutical composition, wherein the method comprises administering the pharmaceutical composition to a subject in need thereof, and measuring and/or monitoring the bioactive brain health protective efficacy. The method comprises measuring and monitoring the levels of biomarkers of brain function such as BDNF, NGF, AChE, ChAT, inflammatory markers, markers of the Wnt/beta catenin pathway, such as Dkk-1 and combinations thereof.

Described herein is a method of treating a subject in need thereof and promoting or protecting brain health of the subject, the method comprising identifying a subject diagnosed with one or more diseases selected from the group consisting of hypercholesteremia, metabolic syndrome, type II diabetes, obesity, osteopenia, osteoporosis, hypertension and post menopausal women on hormone replacement therapy and combinations thereof, and administering an effective amount of the disclosed pharmaceutical composition to the subject to treat the one or more diseases and to protect or promote the brain health of the subject.

Described herein is a method of treating an individual with one or more of hypercholesteremia, metabolic syndrome, type II diabetes, obesity, osteopenia, osteoporosis, hypertension and post menopausal hormone replacement therapy, wherein the method comprises administering individualized dosages of the disclosed pharmaceutical composition to a subject in need thereof in addition to the specific treatment for their primary disease.

Described herein is a method of activating alpha-secretase activity, wherein the method comprises administering an effective amount of the disclosed pharmaceutical composition to cells associated with the processing of APP or to a subject in need thereof.

Described herein is a method of inhibiting beta secretase activity and/or the gamma secretase activity, wherein the method comprises administering an effective amount of the disclosed pharmaceutical composition to cells associated with beta secretase activity and/or the gamma secretase activity or to a subject in need thereof.

Described herein is a method of inhibiting accumulation of beta amyloid in the brain of a subject, wherein the method comprises administering an effective amount of the disclosed composition to a subject in need thereof.

Described herein is a method of promoting efflux of soluble non-amyloidogenic amyloid precurson protein metabolites in a subject, wherein the method comprises administering an effective amount of the pharmaceutical composition to a subject in need thereof.

Described herein is a method of inhibiting phosphorylation of tau protein in a subject, wherein the method comprises administering an effective amount of the disclosed pharmaceutical composition to a subject in need thereof. The subject can be a subject diagnosed with AD.

Described herein is a method of inhibiting inflammation in a subject, wherein the method comprises administering an effective amount of the disclosed pharmaceutical composition to a subject in need thereof.

Described herein is a method of inhibiting cytokine levels in the brain of a subject, wherein the method comprises administering an effective amount of the disclosed pharmaceutical composition to a subject in need thereof.

Described herein is a method of inhibiting oxidative stress in a subject, wherein the method comprises administering an effective amount of the disclosed pharmaceutical composition to a subject in need thereof.

Described herein is a method of inhibiting neuronal apoptosis in a subject, wherein the method comprises administering an effective amount of the disclosed pharmaceutical composition to a subject in need thereof.

Described herein is a method of modulating NMDA receptors in a subject, wherein the method comprises administering an effective amount of the disclosed pharmaceutical composition to a subject in need thereof.

Described herein is a method of inhibiting glutamate toxicity in a subject, wherein the method comprises administering an effective amount of the disclosed pharmaceutical composition to a subject in need thereof.

Described herein is a method of protecting and maintaining the blood brain barrier, wherein the method comprises administering an effective amount of the disclosed pharmaceutical composition to a subject in need thereof.

Described herein is a method of neuroprotection of the brain of a subject, wherein the method comprises administering an effective amount of the disclosed pharmaceutical composition to a subject in need thereof.

Described herein is a method of promoting neurogenesis in a subject, wherein the method comprises administering an effective amount of the disclosed pharmaceutical composition to a subject in need thereof.

Described herein is a method of promoting expression of one or more proteins associated with neurogenesis in a subject, wherein the method comprises administering an effective amount of the disclosed pharmaceutical composition to a subject in need thereof, and wherein the one or more proteins are selected from the group consisting of BDNF, NGF, BMP, Shh, Notch and combinations thereof.

Described herein is a method of activating wnt/beta catenin signaling pathway in a subject, wherein the method comprises administering an effective amount of the disclosed pharmaceutical composition to a subject in need thereof.

Described herein is a method of inhibiting Dkk-1 in a subject, wherein the method comprises administering an effective amount of the disclosed pharmaceutical composition to a subject in need thereof.

Described herein is a method of inhibiting GSK-3 beta antagonist in a subject, wherein the method comprises administering an effective amount of the disclosed pharmaceutical composition to a subject in need thereof.

Described herein is a method of enhancing neurotransmission in a subject, wherein the method comprises administering an effective amount of the disclosed pharmaceutical composition to a subject in need thereof.

Described herein is a method of stimulating acetylcholine synthesis in a subject, wherein the method comprises administering an effective amount of the disclosed pharmaceutical composition to a subject in need thereof.

Described herein is a method of stimulating acetylcholine transferase activity in a subject, wherein the method comprises administering an effective amount of the disclosed pharmaceutical composition to a subject in need thereof.

Described herein is a method of inhibiting cholinesterase activity in a subject, wherein the method comprises administering an effective amount of the disclosed pharmaceutical composition to a subject in need thereof.

Described herein is a method of stimulating serotonin synthesis in a subject, wherein the method comprises administering an effective amount of the disclosed pharmaceutical composition to a subject in need thereof.

Described herein is a method of stimulating synthesis of insulin in the brain of a subject, wherein the method comprises administering an effective amount of the disclosed pharmaceutical composition to a subject in need thereof.

Described herein is a method of stimulating the wnt/beta catenin pathway in a subject, wherein the method comprises administering an effective amount of the disclosed pharmaceutical composition to a subject in need thereof.

Described herein is a method of stimulating the binding of beta catenin to its receptor in a subject, wherein the method comprises administering an effective amount of the disclosed pharmaceutical composition to a subject in need thereof.

Described herein is a method of stimulating the synthesis of beta catenin in a subject, wherein the method comprises administering an effective amount of the disclosed pharmaceutical composition to a subject in need thereof.

Described herein is a method of stimulating synaptic transmission in a subject, wherein the method comprises administering an effective amount of the disclosed pharmaceutical composition to a subject in need thereof.

Described herein is a method of inhibiting accumulation of oxygen radicals in brain of a subject, wherein the method comprises administering an effective amount of the disclosed pharmaceutical composition to a subject in need thereof.

Described herein is a method of enhancing supply of oxygen and/or glucose to the brain of a subject, wherein the method comprises administering an effective amount of the disclosed pharmaceutical composition to a subject in need thereof.

Described herein is a method of enhancing cerebral blood flow in a subject, wherein the method comprises administering an effective amount of the disclosed pharmaceutical composition to a subject in need thereof.

Described herein is a method of promoting insulin sensitivity in the brain of a subject, wherein the method comprises administering an effective amount of the disclosed pharmaceutical composition to a subject in need thereof.

The methods described herein can involve comparing the results of a subject at risk or diagnosed for a disease or condition with the results of a known healthy subject. Likewise, the methods of promoting, enhancing, stimulating, inhibiting, reducing, or decreasing the levels of one or more specific factors in a subject or cells can involve comparing with a control, wherein the control has a known result or is a known healthy subject, or known healthy cells.

Described herein is a method of assuring that an individual subject in need thereof has bioactive levels of the components of the disclosed pharmaceutical composition in their blood, the method comprising administering the disclosed pharmaceutical composition to the individual subject and measuring and/or monitoring the absorption of bioactive levels of the components of the disclosed pharmaceutical composition and comparing the measured and/or monitored levels of the three active components with: (1) a predetermined baseline level of each active ingredient for the individual subject; and (2) optimal bioactive levels of each active ingredient to determine if there has been a positive change in levels compared with the baseline and whether the levels fall within the optimal bioactive levels or ranges and if the results show that there has not been a positive change with respect to the baseline levels and/or the levels are not within the optimal bioactive levels or ranges, then adjusting and/or supplementing the administration of each active ingredient until a favorable change with respect to the baseline levels and/or levels or ranges within the optimal bioactive levels are achieved.

The subject can be a mammal. The mammal can be a human, a rat, a mouse, a dog, or a pig. The subject is in need of treatment or in need of the administration of the nutraceutical composition. The subject is a patient in need of treatment or in need of administration of the pharmaceutical composition. The subject or patient can be a female and the methods described herein can be used to treat, prevent, or monitor a female subject.

As described herein lower doses are provided for promoting healthy brain aging while higher dosages are provided to the cognitively impaired, for example subjects having mild cognitive impairment or suffering from AD. The lower dosage can be formulated as a nutraceutical composition, while the higher dosage can be formulated as a pharmaceutical composition.

Described herein are methods of treating female subjects at risk for developing mild cognitive impairment or AD comprising administering the disclosed composition. The methods could be combined with disease specific therapies such as for diabetes, obesity, osteopenia, osteoporosis, hypertension, cardiovascular disease and combinations thereof. Other diseases and conditions include metabolic syndrome, neuronal damage, post concussion, PTSD, stroke, Huntington's disease, schizophrenia and combinations thereof.

Biomarkers, such as inflammatory markers or growth factors, are used to determine the absorption of the components of the disclosed composition for adjustment of the dosages as needed to aid in long term treatment of asymptomatic women. The increase or decrease in the presence of a particular biomarker is compared with the level of the same biomarker in a known healthy women or a known level in a healthy woman.

Described herein is a method of making the composition comprising mixing the components of the composition to form the composition.

Described herein is a kit comprising the composition, wherein the kit comprises the components in effective amounts for treatment or prevention of disease or condition or for monitoring and/or measuring the components of the composition for determining whether a subject is being effectively treated.

### BRIEF DESCRIPTION OF THE DRAWINGS

FIG. 1: Structures of Estradiol and Isoflavones. FIG. 1 shows similarity in the molecular structures of the predominant natural estrogen in women, 17-beta estradiol, and the two principle estrogens in soy isoflavone extract: genistein and daidzein.

FIG. 2: Neuronal Estrogen and Phytoestrogen Signaling Pathways. FIG. 2 shows metabolic bioconversion of both estradiol and isoflavone phytoestrogens when consumed orally. Estradiol is converted into estrone by the liver and then via 17 beta hydroxysteroid dehydrogenase activity, metabolized in peripheral tissue into bioactive estradiol. Isoflavone glycosides (daidzen and Genistin) are absorbed in the gastrointestinal tract, where the glucoside moiety converts both into bioactive aglycone molecules (daidzein; genistein). All have variable affinity for the estrogen receptors alpha and beta and the membrane GPR 30 receptor.

FIGS. 3A and 3B: Genistein Induces Phosphorylation of Estrogen Receptor and Genistein Increases Protein Expression of Neurotrophic Factors. FIG. 3 shows genistein has a comparable rapid—albeit-slightly attenuated ability to phosphorylate the estrogen receptor when compared with 17-beta estradiol. Genistein increases the protein expression of the neurotrophins—BDNF and NGF.

FIG. 4: High-soy diets increase dendritic spine density in the hippocampus and prefrontal cortex of rats and improve memory for spatial location (Luine et al). Surgically menopausal adult female rats fed high phytoestrogen diet show enhanced spine density in brain regions subserving memory (top graph) and enhanced memory for the placement of objects (lower graph) compared with controls fed low phytoestrogen diets for 7-9 weeks. *** p<0.001 for higher spine density in hippocampus versus prefrontal cortex; ** p<0.01 for greater spine density with high versus low phytoestrogen diet; * p<0.05 for enhanced memory for object placement with high versus low phytoestrogen diet (Luine et al).

FIG. 5: Soy supplements improve performance on executive function tasks in postmenopausal women (Duffy et al). In a clinical trial, postmenopausal women (mean age 50-65) randomized to receive 60 mg total isoflavones/day from a soy isoflavone supplement (Solgen) showed significant improvements on two executive function tests, the IDED (Intradimensional/Extradimensional Shift; a test of mental flexibility) and the SoC (Stockings of Cambridge; a test of planning ability), compared with women randomized to receive placebo.** p<0.01 for enhanced performance with soy supplementation. * p<0.05 for enhanced performance with soy supplementation (Duffy et al).

FIG. 6: Genistein Increases Expression of BDNF and NGF: comparison with 17-beta estradiol (Xu et al). Genistein increases expression of the neurosteroids, BDNF and NGF, in a dose dependent manner, with the highest dose, having a lesser effect than that following treatment with 17-beta estradiol. Inhibiting the estrogen receptor blocks the expression of both BDNF and NGF.

FIG. 7: Neuronal Estrogen and Neurosteroids. Endogenous synthesis. The metabolic pathway for the endogenous synthesis of the estrogen and androgen sex steroids peripherally and in the brain, and their respective bioactive metabolites.

FIG. 8: Plasma huperzine A concentrations obtained after a single ER capsule etc.

FIG. 9: Predicted plasma huperzine A concentrations.

FIG. 10: Brain aging and Pathophysiologic Changes in Molecular & Cellular pathways. An overview of the layered and multiple molecular and cellular pathways influencing brain aging, with the sites of the disclosed composition's modulating“check and balance” of individual ingredient bioactivity.

FIG. 11: Complementing Non-amyloidogenic Metabolism of APP.

Complementing and “balanced” pathways stimulating the non-amyloidogenic metabolism of amyloid precursor protein by the components of the disclosed composition, with an enhanced excretion of soluble beta amyloid metabolites.

FIG. 12: Single Pathway Brain Health Modulators vs Multiple Site Activity of the Components of the Disclosed Composition: Neuronal Glucose Insulin Imbalance. Factors involved in neurodegeneration associated with brain insulin resistance, and a comparison of the single pathway modulation of marketed treatments for Azlheimer's Disease compared to the multiple pathway of the disclosed composition's sites of bioactivity.

FIG. 13: Balancing the regulation of Wnt/beta catenin and Dkk1. An overview of the Wnt/beta catenin glycoprotein pathway, resulting in the binding of beta catenin to an intranuclear T-cell factor thus initiating the transcription of the brain cells target genes and function. Sites of the disclosed composition's stimulation of the Wnt signaling and its “balancing” inhibition of three Wnt inhibitors: Dkk1; GSK-3 beta; Acetylcholinesterase.

FIG. 14: Wnt/beta-catenin: Correlating Osteopenia with Risk for Alzheimer's Disease. Women with osteoporosis are at increased risk of developing Alzheimer's disease. Both the brain and bone have similar Wnt driven pathways that can either result in increased neurogenesis or new bone formation respectively. An increase in Dkk1 inhibitory activity (middle panel) produces an imbalance in the both pathways with potential neuropenia (cognitive impairment) osteopenia (fracture risk). Vitamin D and estrogen have positive agonist effects.

FIG. 15: Adult Neural Stem Cell Neurogenesis. Adult stem cell neurogenesis takes place throughout adult life in the subgranular zone of the hippocampal dentate gyrus, and in the subventricular zone of the lateral ventricle. These complex neural pathways are regulated by a number of integrated growth factors and neurotrophins in an environment of physiologic hypoxia. These pathways are positively modulated by the bioactivity of the disclosed composition's ingredients and additives.

### DETAILED DESCRIPTION

### Compositions

The present application recognizes the need to define the multiple neurologic pathways involved and to formulate combinations of natural ingredients that address and “normalize” the physiologic metabolic changes associated with brain aging per se from that of an underlying latent neurologic disease. Successful management requires viable neurons, and thus the need for early recognition (health promotion) and treatment (disease prevention) of the underlying disorder.

The present application applies the bioactivity of its combined ingredients to a number of established and inter-related molecular pathways that govern the function of the aging mammalian brain thus promoting both healthy brain aging and the prevention and/or inhibition of neurodegenerative conditions and certain neuropsychiatric disorders, with special application to cognitive, memory and mood dysfunction.

Huperzine has been reported to have selective and long-term inhibition of brain AChE with few side effects (Tang, Acta Pharmacol. Sinica 17:481 (1996)). Estrogen has been shown to reduce the incidence of Alzheimer's disease (AD) and related dementias, relieve symptoms of Alzheimer's disease, preserve cholingergic function, and improve cognitive function in post menopausal women and in patients with Alzheimer's diseases (Sherwin, Ann. NY Acad. Sci. 743:213 (1994)).

The present application provides compositions comprising Huperzine A or a derivative or analog thereof, one or more phytoestrogens, and a vitamin D. The composition can include additives. The composition can be a pharmaceutical composition or a nutraceutical composition. The present application provides methods of administering to a subject or promoting brain health and/or preventing neurodegnerative conditions. The subject is a patient in need of treatment or in need of administration of the pharmaceutical composition such as, for example, a subject exhibiting one or more symptoms associated with brain aging, a neurodegenerative condition and/or a neuropsychotic disorder.

Described herein are methods of treating subjects in need thereof or in need of treatment, wherein the disclosed compositions are administered to the subject once daily or twice daily. In one embodiment, the disclosed compositions are administered twice daily for immediate release. In another embodiment, the disclosed compositions are administered once daily for extended release.

The components of the disclosed compositions can be provided, for example, in amounts and/or in a sequence or order to act synergistically to provide enhanced effects. The effects can be therapeutic and enhanced as compared to a composition consisting essentially of Huperazine A and an estrogen, such as a phytoestrogen. The effects are enhanced about two times, about five times, about 10 times, about 20 times, or more as compared to a control composition consisting essentially of Huperazine A and an estrogen or phytoestrogen.

As an example of the components working synergistically and complementarily, genistein, a phytoestrogen, increases the production of vitamin D receptor and promotes its in situ synthesis while decreasing its catabolism. Huperazine and vitamin D are known to increase the production of NGF and soy estrogen and caffeine are known to increase the production of BDNF.

The components of the disclosed composition can be a natural or endogenous product or a synthetic product or combinations thereof.

Provided herein are compositions comprising a combination of botanicals and natural compounds, each of which have validated and experimentally proven biologic efficacy in improving and/or modulating relevant physiologic metabolic pathways associated with the recognized alteration in memory, cognitive and executive function in aging adult women. The composition and formulation disclosed herein addresses the optimization of normal “healthy” brain aging (health promotion) and also changes associated with “unhealthy” brain aging including: women with pre-existing risk factors for cognitive and related dysfunction and those who are predisposed to or have early evidence and/or symptoms of the pathologic features associated with mild cognitive impairment and Alzheimer's Disease (prevention).

The disclosed composition and formulation can also be provided as a nutraceutical complement for use together with marketed drug therapy for cognitive dysfunction and memory loss and as an adjunct with drugs used to treat conditions that are recognized risk factors for AD. This includes drugs for the treatment of type II diabetes, hormonal therapy for post menopausal women, lipid lowering drugs for hypercholesteremia and for obesity, drugs for treatment of the metabolic syndrome, drugs for treating osteoporosis and combinations thereof.

The composition described herein comprises a core of three ingredients: a blend of huperzine A; soy isoflavones and vitamin D, such as vitamin D3 (1,25-(OH)2 D3). Each have similar and/or complementing efficacy on the cellular physiology, function and neurologic pathways relative to memory, cognition and executive function. (Also referred to as a “Broad Based Balanced Bioactive Brain Blend”™—BBBBB™.)

Moreover, one or more additives can be added to the disclosed composition to form a “blend” to address specific medical conditions and/or clinical preference and/or choice of consumption. Examples of two additives are: caffeine and sucromalt (Cargill, Xtend®)—a nutritive low-glycaemic sweetener.

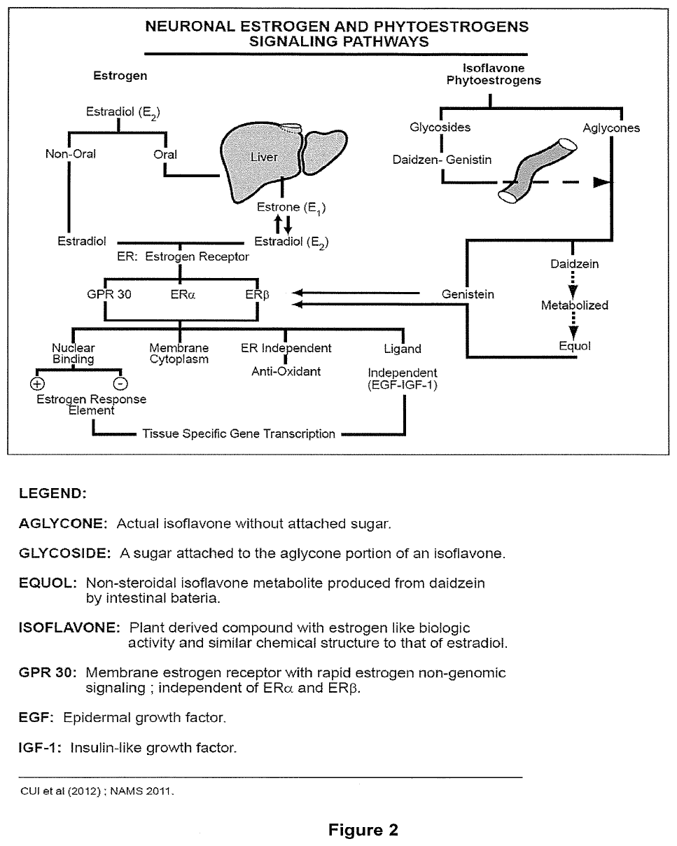

Exemplary combinations are formulated to take account of their individualized and combined pharmacokinetic and phamacodynamic profiles and are adjusted to meet the clinical intent of promoting brain health and/or preventing cognitive and related neurologic dysfunction. Exemplary embodiments provide combination products with additive, synergistic and/or complementary function. The latter addresses both sides of the “checks and balance” associated with many biologic functions. For example, Huperzine A prevents the breakdown of acetylcholine and soy isoflavones (genistein) up regulates its synthesis (see FIGS. 2, 3, and 4 in U.S. Pat. No. 6,524,616).

### Huperzine A

The composition described herein comprises Huperzine A. The Huperzine A can be an analog and derivative thereof. The Huperzine A, including its analogs and derivatives thereof, can be a synthetic molecule. Huperzine A (HupA) is a well-described and researched natural cholinesterase inhibitor (Wang et al). HupA inhibits acetylcholinesterase (AChE) in the cerebral cortex and importantly in the hippocampus. Acetylcholine synthesis is markedly reduced in AD (Wang et al).

Huperzine A is a novel Lycopodium alkaloid that was first isolated from the Huperzia serrata Trev and Chinese folk herb Qian Cheng Ta. It is a potent and selective brain AChE inhibitor with greater potency and fewer side effects than other currently-available AChE inhibitors. The lack of systemic side effects is attributed to HupA's negative effect on the systemic acetylcholinesterase inhibitor, butyrylcholinesterase (BuChE).

Although HupA is unable to retard neurodegeneration in patients with established AD, it does have properties that stimulate neurogenesis; provide neuroprotection; stimulate neurotransmission and most importantly regulate beta amyloid precursor protein (APP) metabolism and in so doing lessen the accumulation of both beta amyloid plaques and tau neurofibrillary tangles. The key to HupA's protective potential is its early use, so that viable and responsive neurons are still available to respond both to and with other co-administered neuroprotecting compounds.

Neurotransmitter Activity: HupA produces a more prolonged increase in ACh when compared with all other cholinesterase inhibitors. Although there is a regional variation, the maximal increase occurs in areas associated with memory and cognition: frontal and parietal cortex and the hippocampus. The time course of cortical AChE inhibition with HupA mirrors the increase in ACh at the same dose, thus confirming that the increase in extracellular ACh is primarily due to the inhibition of cortical AChE.

Brain norepinephrine (NE) and dopamine (DA) levels are also increased following systemic administration of HupA but not serotonin (5-HT). The effect is greater for DA than it is for NE. It is postulated that the effect of HupA on DA and NE is regulated by presynaptic ACh muscarinic and/or nicotinic receptors, thus contributing to the memory improvement following treatment with HupA (Wang 2006). Protection against glutamate-induced cytotoxicity: HupA protects against glutamate induced cytotoxicity. This was demonstrated in rat hippocampal neuronal cells. In a dose dependent manner, HupA acted as a non-competitive and reversible inhibitor of the NMDA receptors, via a competitive interaction with polyamine binding sites (Zhang and Hu 2001).

Neuroprotection: Plaques characteristic of AD are caused by the deposition of beta amyloid and are typical of lesions found in the brains of patients with AD. This process is initiated in part by oxygen radicals that leads to neurodegeneration. HupA protects against H₂O₂ by increasing antioxidant enzymes (Zhang et al 2002); HupA protects against cellular damage when exposed to oxygen-glucose deprivation (OGD) by alleviating the disturbances of oxidative and energy metabolism (Zhou et al 2001); HupA reduces oxygen free radicals in both animal experiments and clinical trials (Shang et al); HupA provides neuroprotection by modulating the intracellular Ca++ level including the transcription of calmodulin in hippocampal neurons (Lu et al 2004); decreasing apoptosis of neural cells after exposure to stressors such as H2O2; beta amyloid peptides and OGD are significantly reduced following administration of HupA and with the normalization of the anti-apoptopic Bcl-2 genes with attenuation of the pro-apoptopic Bax and P53 genes (Xiao et al 2002; Wang 2006); finally, HupA protects mitochondrial activity. In summary, the neuroprotective effect of HupA is achieved via multiple mechanisms.

Neurogenesis: The regulation of nerve growth factor (NGF) synthesis and its release is governed via cholinergic mechanisms. HupA increases the NGF regulated enhancement of neuron survival and function probably via its inhibition of AChE, as shown by the associated neurite outgrowth with the level of AChE expression (Tang et al 2005). NGF and its TrkA receptor mediate the neuroprotective actions of HupA (Wang et al 2006).

Amyloid Precursor Protein Processing: Beta amyloid is derived from a larger polypeptide amyloid precursor protein (APP). There are two pathways for the processing of APP: a non-amyloidogenic end point which is modulated via a SIRT1 directed gene encoding alpha secretase pathway. This cleaves the APP away from the toxic beta amyloid peptide and also reduces tangle formation by deacetylating tau. Metabolism via the beta and gamma pathways has the reverse effect: an increase in both extracellular beta amyloid neuronal plaque formation and intracellular tau tangles (Guerente 2011). HupA directs APP metabolism toward the non-amyloidogenic alpha secretase pathway (Peng et al 2007).

Pharmacokinetics: HupA is rapidly absorbed, is widely distributed in the body and is eliminated at a moderate rate. The elimination of HupA in elderly volunteers is slightly lower than that in the younger subjects. The definitive pK study for HupA was published in 2008 (Li et al 2008). Healthy subjects received 0.2 mg of pure huperzine A orally. Plasma levels rose rapidly after administration peaking at about hour 1.2 to 1.3. The plasma levels declined rapidly over the next 24 hours and a terminal half life of approximately 6 hours was determined. Over the major part of the day plasma levels ranged from 0.3 to about 1.0 ng/ml where 0.6 ng/ml was determined to be the optimal level. Values above this concentration produce unnecessary exposure of tissues to brief high levels of huperzine and an increased loss due to excretion. Doses of 0.15 mg twice daily has been shown to be effective for the treatment of MCI (Du et al 1996).

Based on the above data, a controlled-release formulation of HupA would be required for a once a day administration.

Clinical Studies: The efficacy and safety of HupA have been studied in a number of clinical trials, principally in China and some in the US (Wang et al 2006; Little et al 2008). Most of the studies were conducted in patients with established AD. In one of the larger trials involving some 819 patients with AD, treatment with HupA in a dose of 0.03-0.4 mg/day resulted in an improvement of their memory, cognitive skills and activities of daily living. Another double blinded randomized clinical trial evaluated patients with possible or probable AD taking 0.1-0.2 mg of HupA twice daily. Cognitive function was measured with the MMSE (Mini-mental State Examination Scale), the ADAS-Cog (Alzheimer's Disease Assessment Scale-Cognitive Subscale), the ADAS-non-Cog (which measures mood and behavior and activities of daily living (ADL). All showed significant improvement at week 6 and further improvement at week 12. The proportion of patients with a four point improvement on the ADA Scog was 56% in the active group and 12.5% in the placebo group (Zang et al 2002).

A longer term study extending over 48 weeks confirmed significant improvement in cognition at all time points (Wang et al 2006).

There have been fewer studies in the US (Little et al 2008). As with the trials in China, the use of HupA (in doses as high as 200 mcg b.i.d. and even 400 mcg b.i.d.) confirmed its ability to be pharmacologically effective (inhibiting AChE levels in all tested subjects by 50% or more without any significant BuChE inhibition) and clinically safe. However, the reported clinical improvement was not as robust as that noted in Chinese literature.

Summary: The mixed HupA clinical trial results may be due to differences in the populations studied and to the presence and extent of existing neurologic damage in the chosen test subjects. HupA cannot reverse the function of significantly damaged neurons, characteristic of patients with well defined clinical AD.

Given HupA's broad range of experimentally proven brain protective mechanisms, the composition described herein is designed to be used in women with functionally responsive neurons. This includes women who are asymptomatic and otherwise healthy, post menopausal women with cognitive and memory complaints and for two additional categories: women with risk factors for AD and women with symptoms of early MCI. In each instance, formulations will include additional bioactive brain health promoting compounds and the doses of each adjusted according to the clinical indication for the use of the disclosed composition and according to individual patient's response.

### Estrogen

The composition described herein comprises estrogen in addition to Huperzine A and vitamin D. The estrogen can be selected from the group consisting of estradiol, conjugated equine estrogens (CEE), any active estrogenic ingredients of CEE, estrone, estriol, esterified estrogens, and any derivative, analog, or metabolite of the mammalian estrogen and combinations thereof. The estradiol is 17-beta estradiol, estradiol valerate, ethinyl estradiol, or any other estradiol derivative or analog, or metabolite thereof. The estrogen can be natural or endogenous molecule, or a synthetic molecule. The natural or endogenous molecule can be from a mammalian source. The estrogen can be an analog or derivative, and the analog or deverivature may be a natural or synthetic molecule.

Estrogen is known to have neuroprotective effects and cognitive function. Estrogen promotes brain health and protects cognition in both perimenopausal and postmenopausal women, provided the estrogen therapy (ET) in the latter group is initiated close to the time of menopause. Early treatment modulates the compromising neurobiological changes associated with “normal” aging (Voytko et al 2009; Maki 2006; Gilles and Mcarthur 2010). The majority of observational studies confirm that estrogen users perform significantly better than non users on tests of verbal fluency, verbal memory, and spatial working memory (Sherwin and Henry 2008).

The age related difference in ET response may be due to the recognized alteration in estrogen receptor (ER) amount, distribution, integrity and post receptor signaling pathways found in aging blood vessels and brain (Smiley and Khalil 2009; Gilles and Mcarthur 2010). Hence the need for early intervention.

Brain imaging: Studies have shown that women on hormonal therapy had larger hippocampi compared to non users (Lord et al 2008) greater grey and white matter volume (Erickson et al 2005), and less shrinkage of cortical tissue over 5 years (Raz et al 2004).

Hormonal users also had increased cerebral blood flow and connectivity in areas related to cognition and memory: frontal-temporal cortex and hippocampus (Maki and Resnick 2000; Ottowitz et al 2008)

Neurogenesis: Numerous mechanisms have been identified including the proliferation of human cortical neural progenitor cells (Brinton 2009); spinogenesis and the regulation of dendritic spine number and contacts via multiple synaptic boutons (Lamprecht and Le Doux 2004). An increase in spine density in the hippocampus is associated with enhanced learning and memory (Lamprecht and Le Doux 2004). Conversely, decreased synapse density and synaptic dysfunction precede AD (Shankar et al 2008)

Neurotransmission: Estrogen regulates the synaptic plasticity and the genesis of new circuits potentiating synaptic transmission via glutamate and NMDA receptors (Woolley 2007). Recent studies in women have also demonstrated that the acetylcholine (Ach), dopaminergic and serotonergic systems of neurotransmission are all responsive to hormonal therapy (Voytko et al 2009).

Of particular relevance, estrogen deficiency results in decreased choline acetyltransferase (ChAT) activity, and ChAT, brain derived neurotrophic factor (BDNF), and nerve growth factor (NGF) mRNA's all of which can be reversed with estrogen supplementation (Luine, V N 1985; Gibbs R B et al 1994; Singh M et al 1995; Singh M et al 1994; Sohrabji F et al 1995).

Estrogen receptors and estrogen synthesis: More recently, in addition to the two classically identified estrogen receptors—ER alpha and ER beta—a third membrane receptor has been identified—GPR30. Also a new family of coregulatory proteins and the discovery of brain (local as opposed to peripheral) estrogen synthesis via a calcium dependent phosphorylation of an aromatase enzyme, and its effect—especially on the rapid response to estrogen—of neurotransmission (Charlier et al 2010).

Estrogen and brain glucose regulation: Estrogen up regulates the production of the glucose transporter GLUT1 in both the endothelial tissues and in the cerebral cortex (Dormire 2009; Cheng et al 2001). Estrogen also increases insulin sensitivity.

### Phytoestogens and Isoflavones

The composition disclosed herein comprises phytoestrogen in addition to Huperzine and vitamin D. The composition can also include an estrogen in addition to phytoestrogen.

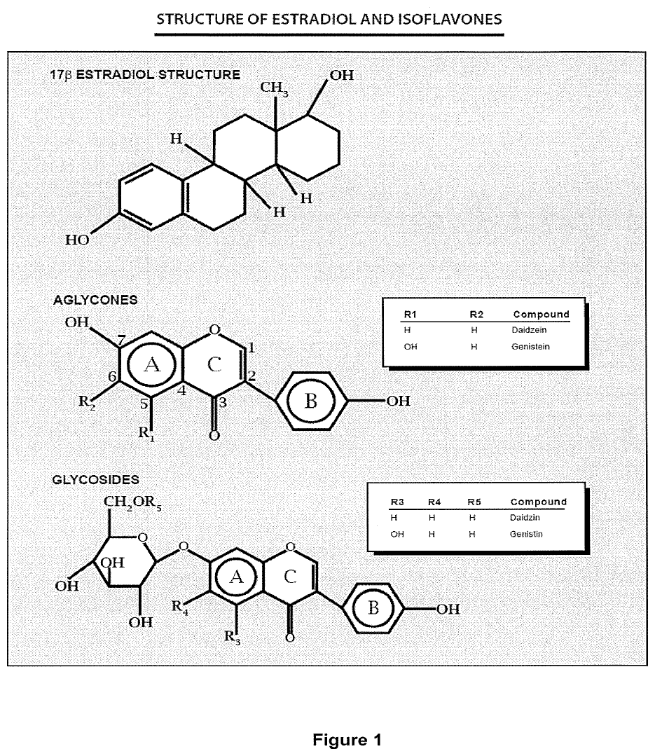

Structure and source: Phytoestrogens are natural compounds found in many plants and have estrogen like activity in mammals including humans. There are two chemical categories—coumestans and isoflavones—with a molecular structure similar to the 17-carbon structure of estradiol (FIG. 1). The phytoestrogen can be selected from the group consisting of an isoflavone, a coumestan, a lignan, analogs and derivatives thereof, and combinations thereof. The phytoestrogen can be a natural or endogenous molecule or a synthetic molecule.

Isofavones include the bioactive constituents: genistein, daidzein, glycitein, biochanin A and formononetin. Genistein and daidzein, the main components in the disclosed composition, are found in high concentrations in soybeans and soy products and also red clover, kudzu and the American groundnut. Dietary phytoestrogens are efficiently absorbed from the gastrointestinal tract. Genistein, daidzein and equol are the main absorbed metabolic products of isoflavones, generated by colonic bacteria that remove a glycoside moiety (King et al 1998; Clarkson et al 2011). The relative amounts of genistein and daidzein are the main determinants of the bioactive components of soy supplementation, although the therapeutic outcome may vary when the individual isoflavones are administered alone or in combination. Two other efficacy variables are: an individuals ability to metabolize daidzein to equol. This occurs in about 30% of American women. Equol binds to both ER's but has a particular affinity for the ER beta; soy protein is the second factor and is derived by extracting it out of the whole soy bean. Soy protein is usually rich in isoflavones (Clarkson et al 2011). The biological effects of isoflavones and their metabolites are mediated via many pathways some of which are not estrogen-dependent (FIG. 2).

Isoflavones and cognitive function: Soy phytoestrogens act as estrogen agonists and have protective effects on neurons, including cholinergic neurons via ER's alpha and beta. Genistein has a high affinity for ER beta, that is similar to endogenous circulating 17-beta estradiol, but an affinity for ER alpha that is 20 times lower than that of estradiol (Kuiper et al 1997).

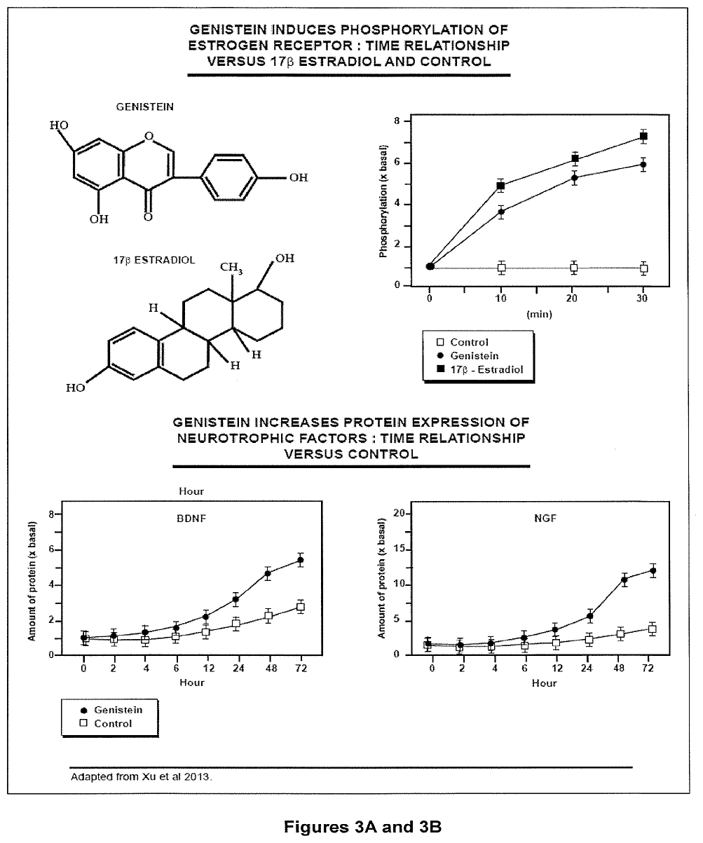

Soy phytoestrogens up-regulate the mRNA levels of choline acetyltransferase (ChAT), an enzyme linked to acetylcholine synthesis and cholinergic function) and also BDNF (Pan et al 1999). Since both ER alpha and ER beta mRNA's are present in the frontal cortex and hippocampus, and are responsive to both estradiol and soy phytoestrogens, it has been established that soy phytoestrogens via their direct interaction with ER alpha and ER beta preserve cholinergic activity in these regions (Pan et al 1999). (See FIGS. 3A and 3B.)

Genistein up regulates aromatize expression (Fiorelli et al 1999). Brain estrogens have been shown to be lower than normal in women with AD. This may be associated with the observation that aromatase expression is altered in AD brains due to a single nucleotide polymorphism in the CYP 19 aromatase gene (Gilles and McArthur 2010).

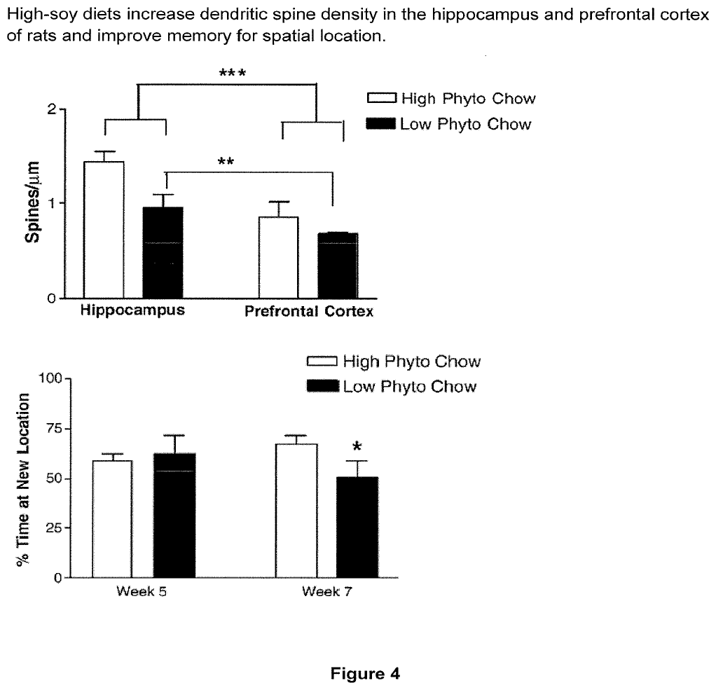

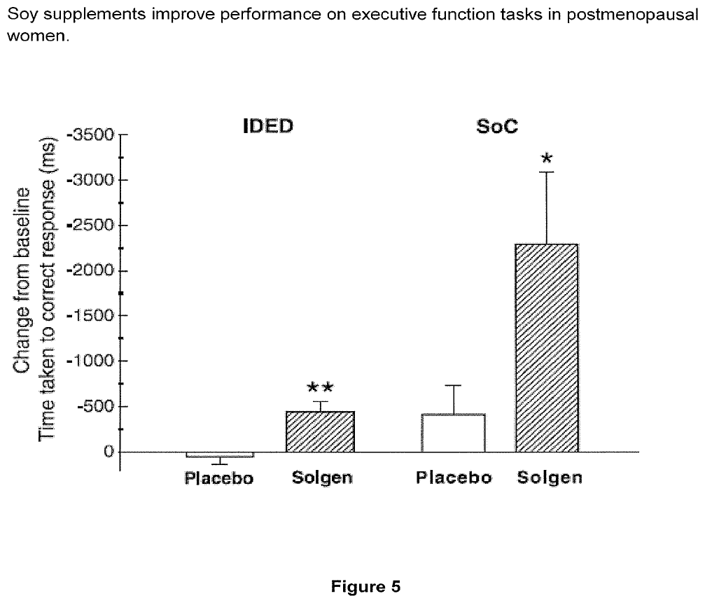

Additional experimental evidence includes an increase in the spine density in the hippocampus and prefrontal cotex of female rats after soy isoflavone treatment that was associated with a significantly greater improvement in spatial memory when compared with placebo treated control rats (Luine et al 2006; see FIG. 4). This experimental data is reflected in a clinical trial of postmenopausal women (mean age 50-65) who were randomized to receive 60 mg total isoflavones/day from a soy isoflavone supplement (Solgen) and showed a significant improvement in two tests of executive function compared to matched controls on placebo (Duffy et al 2003; see FIG. 5).

Soy isoflavones have a number of non-estrogen mediated CNS protective effects: soy phytoestrogens act as aniti-oxidents to protect neurons from oxidative damage and apoptosis (Atlante et al 2010); soy phytoestrogens may increase cerebral blood flow thereby improving the oxygen and nutrient supply to brain cells; genistein inhibits the inflammation and associated endothelial dysfunction implicated in insulin resistance (Gao et al 2013) and by reducing free fatty acids reduces insulin resistance (Lei et al 2011); genistein may actually increase insulin sensitivity by up-regulating the PPAR genes (Ronis et al 2009); genistein has a direct positive effect on pancreatic beta cells and through an effect on cAMP/PKA signaling regulates epigenetic factors associated with type 2 diabetes (Gilbert 2012) and obesity (Behloul and Wu 2013), both of which are risk factors for cognitive decline.

Genistein has anti-inflammatory actions by suppressing tumor necrosis factor alpha induced inflammation by modulating reactive oxygen species/Akt/nuclear factor kB and adenosine monophosphate-activated protein kinase signaling pathways (Li et al 2014).

Genistein up-regulates the vitamin D receptor (VDR) its transcription and expression through the ER and MAPK signaling pathway (Gilad et al 2006); genistein and daidzein increase the expression of CYP27B1 mRNA and suppress CYP24 mRNA expression, the enzymes that respectively activate and deactivate vitamin D synthesis (Gilad et al 2006).

### Pharmacokinetics and Bioavailability.

There is extensive literature on the pharmacokinetics of soy isoflavones administered as natural compounds of soy foods, isolated isoflavone extracts, supplements, pure compounds and also as stable-isotope labeled analogs. Overall, the apparent bioavailability of these isoflavones are similar.

The rates of absorption of the isoflavones daidzein and genestein are distinctly different from those of daidzein in their aglycone form. This determines the ultimate efficacy of isoflavones. Aglycones are rapidly absorbed and reach peak concentrations within 1 to 3 hours, depending on whether the isoflavones are taken with or without a meal. The effect of a meal is to delay absorption and shift the Tmax value. Beta-glycoside conjugate peak plasma concentrations of isoflavones typically occur 4 to 10 hours later due to the need for prior hydrolysis by the intestinal brush border beta-glycosidases. This is a rate limiting and time dependent process.

The t_{1/2} of all isoflavones in healthy subjects is similar and is typically 6 to 12 hours. The clearance rate of genistein is significantly slower than that of daidzein, thus explaining why the plasma concentrations are typically 1.5 to 2.0 times higher than that of daidzein. The extent of isoflavone conjugation varies, with species such as mice having a higher proportion of un-conjugated plasma isoflavones compared to humans. While conjugation can take place in both the liver and enterocytes, the most extensive conjugation occurs by intestinal UDP-glucuronyltransferase on a first pass uptake.

Unlike endogenous estrogens which are extensively bound to sex hormone binding globulin and albumin, both genistein and equol are only 45% to 50% protein bound (Claarkson et al 2011; Bloedon et al 2002; Anupongsanugool et al 2005).

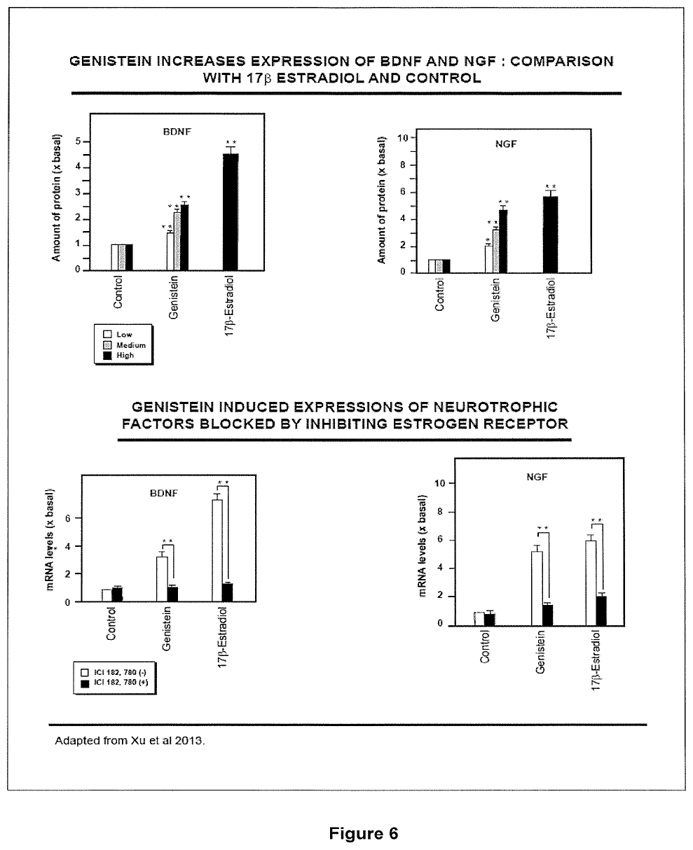

Differentiating the Bioactivity of 17-Beta Estradiol from Genistein (See FIG. 6).

The isoflavone genistein is a plant estrogen that binds to estrogen receptors in both animals and humans, but has two main distinguishing features that are unique to its biologic activity, and to its use—by itself or together with estradiol: genistein (more so than the other isoflavones) has a greater affinity for the ER beta than for the ER alpha receptor, and possesses both estrogen agonist and estrogen antagonist activity. ER beta has a higher affinity for the brain and bone; ER alpha for the breast and endometrium. By down regulating the ER alpha receptor, genistein acts as a SERM (selective estrogen receptor modulator) (Clarkson et al 2011). Genistein is also a tyrosine kinase inhibitor. Together with its anti-proliferative effects, inhibition of angiogenesis and induction of apoptosis, it is “protective” to breast tissue and the endometrium (Clarkson et al 2011).

Dietary isoflavones reduce the circulating and intra-breast concentrations of estradiol in monkeys, with a corresponding decrease in uterine and breast tissue proliferation (Wood et al 2006; Wood et al 2007). This has been confirmed in other animal models. Neither daidzein or equol have been shown to have chemopreventive properties (Lamartiniere et al 2002). These observations are reflective of a number of clinical studies that confirm the life long consumption of soy food and the low prevalence of breast cancer in Asian women (Clarkson et al 2011) and even the lack of tumor promoting effects in breast cancer patients (Shu et al 2009). Dutch women with high circulating genistein levels also had a reduced cancer risk (Verheus et al 2007). Genistein may even have a potential additive/synergistic effects in the chemotherapy of certain cancers: HER 2 over-expressed breast cancer (Seo et al); small cell lung cancer (Zhu et al 2012) and lung adenocarcinoma (Zhou et al 2012).

Genistein and daidzein induce alkaline phosphatase activity in the endometrium, but at one millionth the potency of estradiol (Kayisili et al 2002). As with the breast, a number of large scale studies have correlated high intakes of soy isoflavones with lowered endometrial cancer risk (Clarkson et al 2011). Studies in both normal post menopausal women and women with a history of breast cancer noted neither an increase in Ki-67 expression (a measure of endometrial hyperactivity), endometrial thickening or an abnormal change in endometrial histology (Clarkson et al 2011). One 5 year study in women taking 150 mg isoflavones vs. a placebo found that 70% of the treated group had an atrophic or non-assessable endometrium compared with 81% in the control group (Unfer et al 2004). A lower dose of genistein in its aglycone form (54 mg) was as successful as the use of a traditionally prescribed progestin (norethisterone acetate) in reducing endometrial hyperplasia without atypia (Bitto et al 2010).

Twelve weeks of a daily 30 mg dose of synthetic genistein administered to 84 postmenopausal women, did not induce endometrial thickening or hyperplasia (Evans et al 2011).

In the methods described herein, genistein has proven experimental and human clinical data to support and validate its cognitive enhancing and brain health promoting bioactivity and in addition, its lack of adverse effect on estrogen sensitive organs: the breast and endometrium. Provided herein are methods for promoting cognitive health in women at risk of or with a past history of cancer. The methods described herein provide for the use of both low dose estradiol and synthetic genistein, together with synthetic Huperzine A and vitamin D as a treatment for MCI and early AD.

### Epidemiology and Clinical Trials.

Epidemiolgy: The prevalence of AD is lower in women living in Asia and has been attributed to two main factors: a higher lifelong intake of soy protein and isoflavones and a greater ability to produce equol from daidzein. Among older Japanese the daily intake of soy was calculated to be approximately 10 g/day, which when expressed as mean estimates of aglycone equivalents ranged from 30 to 50 mg/day. A similar daily mean soy protein and isoflavone intake was noted in Shanghai (Clarkson et al 2011; Yang et al 2009). By comparison, the estimated isoflavone intakes in Caucasian women in a recent US study averaged <0.5 mg/day compared with >18 mg/day in women of Japanese ethnicity. About 40% of non-Asian women in this study consumed no daidzein or genistein containing products (Huang M H et al 2002).

About 20 to 30% of Western adults will produce equol when fed soy isoflavones which is significantly lower than the 50 to 60% frequency of equol producers reported in adults living in Asia and consuming soy foods (Setchell and Cole 2006). Equol has a high systemic bioavailability and relatively slow plasma clearance, and may explain the greater efficacy of soy studies in Asians compared with those conducted in Western adults (Setchell et al 2002).

Clinical trials: A number of appropriately designed randomized placebo controlled trials are discussed in and allow for the following conclusions (Clarkson et al 2011).

Women <65 years of age: soy and soy isoflavones have a positive effect on a number of cognitive functions including but not limited to working memory, executive function, verbal memory, and figural memory—depending upon the actual domains included in the study design.

Women >65 years of age: the results are mixed and trend to a null effect on cognitive outcome.

Young vs older postmenopausal women: soy improved verbal memory, fluency attention in the early postmenopausal group (ages 50-59) but not in the older group (age range 60-74).

Soy replete diets: Soy supplementation in women with adequate soy isoflavone in their daily diet showed no cognitive benefits in both young and older postmenopausal women (group age range 55 to 76).

These results are consistent with the estrogen therapy (ET) “critical window hypothesis”. Neurons that are healthy and have not been deprived of endogenous estrogen for a significant time, benefit with respect to their survival and function (the healthy cell concept). Conversely, prolonged exposure of unhealthy neurons to estrogens may actually exacerbate existing neuronal damage. This may be due in part to the change in ER expression with aging and their loss of sensitivity to the estrogen ligand (Gilles and McArthur 2010).

### Vitamin D.

The disclosed composition comprises vitamin D in addition to Huperazine A and at least one estrogen and/or phytoestrogen. Examples of vitamin D includes but are not limited to calcitriol, doxercalciferol, paricalcitol, cholecalciferol (vitamin D3), ergocalciferol (vitamin D2), analogs and derivatives thereof, Vitamin D receptor agonists and modulators, and combinations thereof. Vitamin D is a neurosteroid with a defined role in brain function and in various neurological disorders including cognitive decline (Stewart et al 2010; Harmset al 2011). The vitamin D may be a natural or endogenous molecule, or a synthetic molecule.

Vitamin D receptor modulators that have disease specific actions relevant to brain health and to risk factors associated with cognitive impairment eg VS-105, a vitamin D receptor modulator with cardiovascular protective effects (Wu-Wong J R, KawaiM, Chen Y-W, Nakane M. VS-105: a novel vitamin D receptor modulator with cardiovascular protective effects. British J Pharmacol 2011; 164: 551-560).

Similarly, there are a number of new analogs of 1 alpha, 25 (OH)2 D3 (AVD) that have been developed based on their crystal structure with various/differing functional profiles (Carlberg C, Molnar F, Mourino A. Vitamin D receptor ligands: the impact of crystal structures. Expert Opin Ther Pat 2012; 22: 417-435).

Although traditionally regarded as a “vitamin” synthesized in skin from precursor substrates (7-dehydrocholesterol) and from certain vitamin D rich foods, it is now well established that vitamin D is a member of the super family of nuclear steroid transcription regulators, with vitamin D receptors (VDR) present in most—if not all—tissues and organs.

The two way bioconversion of the biologically inert substrate—7 dehydrocholesterol—into active vitamin D3 is mediated by a two step activation involving Vitamin D3, 25-hydroxylase enzyme, and the 25-hydroxyvitamin D3-1alpha-hydroxylase enzymes. Both of these enzyme systems are localized in the brain confirming that the brain activates the vitamin D precursor directly and is not dependent on the plasma levels of 1,25-(OH)2D3 (active vitamin D3-AVD3). This enzymatic bioconversion has been demonstrated in cells essential for cognition and memory including neurons, glial cells, and activated microglial cells. The nuclear functions of the AVD3 are mediated through the expression of the VDR in relevant anatomical areas of the brain: frontal cortex, temporal frontal lobes and hippocampus (Garcion et al 2002).

### Genomics of the VDR and Vitamin D Metabolism:

VDR: The VDR is the mediator of its natural ligand—AVD3—and the latter's multiple cellular growth and differentiating effects. The gene encoding the VDR has several polymorphism that determine its tissue level activity. The longer protein (ff allele) is a less active transcriptional activator than the FF genotype. This translates into the varying efficacy of vitamin D activity in tissues such as muscle, bone and breast tissue and therefore the level of vitamin D supplementation required by individuals (depending on their genotype) for “normal” organ function (Chen et al 2005). This may have similar implications for brain function.

### Balanced AVD3 Metabolism: Synthesis (Formation) and Catabolism (Breakdown).

There are two enzymes of the cytochrome-P450-hydroxylase family that are responsible for the synthesis of vitamin D (25-D3-1 alpha-hydroxylase) and its catabolism (1,25-D3-24-hydroxylase). The respective genes encoding these enzymes are CYP27B1 and CYP24. The balance between the two determines AVD3's ultimate cellular activity. Genistein up regulates CYP27B1 and down regulates CYP24 in both the colon and breast tissue via the beta estrogen receptor (Cross et al 2004). ER beta is the predominant ER isoform in the brain.

### Age and Vitamin D Metabolism:

Although the ability to absorb vitamin D is not altered by aging, its metabolism from sun light exposure to skin is reduced by about 50% from age 20 to 80 years (Holick 2006). Since vitamin D deficiency is strongly correlated with cognitive impairment in the elderly (see later), age adjusted supplemental doses of vitamin D is a necessary to meet the brain's physiologic needs.

### Neuroprotection.

AVD3 regulates the synthesis of nerve growth factor (NGF) (Neveu et al 1994 (a); Cornet et al 1998) and up regulates the synthesis of other neurotrophins: neurotrophin3 (NT3) (Neveu et al 1994 (b)) and glial cell line derived neurotrophic factor (GDNF) (Naveilhan et al 1996). Stimulation of these neurotrophins has been correlated with a neuroprotective effect (Wang et al 2000).

AVD3 modulates neuronal Ca++ homeostatsis by down regulating calcium channels in hippocampal neurons and hence excess excitotoxic insults; AVD3 also modulates calcium activity by inducing the synthesis of Ca++ binding proteins (Brewer et al 2001).

AVD3 inhibits the synthesis of inducible nitric oxide synthase (iNOS). The latter produces NO with the potential to damage both neurons and oligodendrocytes when produced at high levels (Garcion et al 1998; Dawson et al 1996).

By increasing the expression of gamma-glutamyl transpeptidase activity, AVD3 protects the glutathione cycle cross talk between neurons and astrocytes.

The astrocytes anchor neurons to their blood supply, regulate the neuronal chemical environment and recycle synaptic neurotransmitters. They also contribute to the integrity of the BBB (Dringen et al 2000).

Neurotransmission: AVD3 increases choline acetyltransferase (AChE) and hence an increase in brain acetylcholine (ACh) synthesis (Sonnenberg et al 1986).

Down-regulation of microglial activation: Activated microglia play a key role in chronic neurodegenerative disorders. When activated—by the death of neighboring neurons—the microglia promote further death and dysfunction by attacking other neurons and astrocytes. This results from the excess generation of NADPH—a potent generator of superoxide. When combined with nitric oxide, neuronal cells are sensitized to excessive levels of intracellular calcium and glutamate mediated excitotoxicity, resulting in the inability of astrocytes to sequester and metabolize the glutamate with subsequent apoptosis of neurons (McCarty 2006).

Activated microglia also produce a range of inflammatory cytokines including cyclooxygenase (COX-2), that further potentiates the neurons sensitivity to glutamate induced death. The cytokines from activated microglia stimulate the neuronal production of beta amyloid precursor protein (beta APP), and its conversion to beta Amyloid (Ge et al 2002).

The proportion of activated microglia increases as a function of age, and is one factor that explains why chronic neurodegenerative disorders are more common in the elderly (Rozovsky et al 1998).

Microglial cells express the vitamin D receptor with a resultant inhibition of iNOS synthesis and other activating agonists. AVD3 also boosts astrocyte production of glial-derived neurotrophic factor (GDNF) offering another protective mechanism. Dietary doses of AVD3 attenuate microglia activation (Wergeland yet al 2011).

ABC Efflux Transporters and The Blood Brain Barrier (BBB): ATP-binding cassette (ABC) transporters at the BBB are important contributors to the pathogenesis of CNS disorders (Hartz, Bauer 2010). P-glycoprotein, an ATP driven drug efflux transporter is a critical element of the BBB (Miller et al 2006). VDR activation up-regulates P-glycoprotein in the brain capillaries of rat and human brain microvascular endothelia (Durk et al 2012) and may account for the experimental observation that AVD3 enhances the brain to blood efflux of beta A (1-40) through both genomic and non genomic pathways (Ito et al 2011) and also the AVD3 stimulated phagocytosis and clearance of beta Amyloid from the macrophages of patients with AD (Masoumi et al 2009).

### Vitamin D and Insulin Resistance

Pancreatic beta cells express specific cytosolic/nuclear and membrane VDR's. Vitamin D deficiency—at levels below 25 nmol/L—have been linked to an increased prevalence of various metabolic disorders including type I and type II diabetes (Ross et al 2011). Conversely, a meta analysis showed a significant 55% reduction in diabetes and a 51% decrease in the metabolic syndrome (Parker et al 2010) with high serum concentrations of 25 hydroxy vitamin D.

Factors that affect insulin release and resultant insulin resistance include vitamin D associated gene polymorphism involving vitamin D production, transport and action; as a modifiable environmental factor in autoimmune disease (type I diabetes) and through its immunoregulatory function that protects pancreatic beta cells via its anti-inflammatory actions (Sung et al 2012). In addition, there is evidence that vitamin D may stimulate insulin secretion directly, provided calcium levels are adequate (Tai et al 2008). Vitamin D binds directly to the beta cell VDR, and by stimulating insulin receptor expression, enhances insulin responsiveness for glucose transport (Maestro et al 2000). Vitamin D increases bioconversion of pro-insulin which is inactive to bioactive insulin.

Clinical data regarding the benefit of vitamin D supplementation is sparse, but relevant to the multiple pathway approach to health promotion as provided herein, vitamin D—and genistein—have been shown to reduce free fatty acids—an important and common association with peripheral insulin resistance (Inomata et al 1986).

The optimal vitamin D concentration for reducing insulin resistance has been shown to range between 80 to 119 nmol/L (Takiishi et al 2010).

Pharmacokinetics: The pharmacokinetics of vitamin D—a fat soluble and stored hormone—is complex. In short, concentrations of serum 25(OH) after intake of vitamin D3 is biphasic: a rapid increase occurs at low vitamin D3 levels and a slower response at higher concentrations. At typical vitamin D3 dosing, there is a rapid and near quantitative conversion to 25(OH) D which then serves as both the functional status indicator of the nutrient and as its major storage form in the body. At a vitamin D3 concentration—equivalent to a daily input of 2000 IU—the 25 hydroxylase activity becomes saturated and the reaction switches from first to zero order. The constant maximal production of 25(OH)D—irrespective of the precursor concentration of vitamin D3—is probably in excess of metabolic consumption, and is the reason why serum 25(OH)D levels continue to rise as the vitamin D3 dose increases. Based on this explanation, the point at which hepatic 25(OH)D production reaches zero order, constitutes the low end of normal vitamin D status: this has been calculated to be 88 nmol/L and is consistent with the plasma serum levels required for optimal calcium absorption and normal parathyroid hormone homeostasis (Heaney et al 2008).

Epidemiology: numerous population studies have confirmed the relationship between low levels of vitamin D (hypovitaminosis D) and cognitive decline, with reduced executive function and reasoning, in the elderly. This appears to be a universal problem irrespective of the society, race and to a certain extent, the geographic location. Most of the published studies involve women 65 years and older and include subjects from the US (Llewellyn et al 2011), Italy (Llwellyn et al 2010), France (Annweiler et al 2010), England (Llwellyn 2008) including one study that compared African American women with a similar aged cohort of European Americans over age 55 years. The former had significantly lower levels of 25 (OH)D with decreased cognitive performance (Wilkens et al 2009).

Women with vitamin D (25 (OH) D) values less than 50 nmol/L were more likely to have cognitive impairment compared to cohorts with values above 75 nmol/L; plasma 25 (OH) D levels below 25 nmol/L was associated with 40 to 60% or greater risk of cognitive dysfunction.

A recent analysis of 37 studies suggested that values less than 50 nmol/L was associated with poorer cognitive function, and a greater risk of AD (Balion et al 2012).

### Clinical Evidence: Randomized Clinical Trials Vs Applied Translational Medicine.

Vitamin D has an important physiologic role in promoting and maintaining brain health via validated metabolic pathways. The functional effect of vitamin D is complementary and/or additive to the other ingredients of the disclosed composition: Huperzine A and soy isofalvones. These include: factors preventing neurodegeneration; the regulation of neurotrophins (BDNF; NGF); enhancement of acetylcholine neurotransmitter function, insulin sensitivity, BBB protection and the clearance of amyloid beta peptide.

The mechanism(s) underlying the cognitive changes associated with “normal” aging—including the pathogenesis of MCI and AD—are multiple, heterogeneous and evolve over decades of “silent” change.

It is therefore highly unlikely that a meaningful blinded randomized vitamin D alone study, even in an appropriately selected group of early post menopausal “healthy” versus women at risk, will ever meet statistical power and be affordable (Annweiler and Beauchet 2011).

Instead, reliance will need to be placed on surrogate biomarkers that confirm levels of vitamin D consistent with a known brain health effect. These are described below.

### Additives

The compositions and pharmaceutical compositions disclosed herein may comprise one or more additives. Examples of additives include but are not limited to coffee, xanthine alkaloids, chlorogenic acid, and sweeteners. Examples of xanthine alkaloids include but are not limited to cafeine, theobromine, paraxanthine. Examples of sweetener includes low glycemic sweetener selected from the group consisting of sucromalt, tagatose, isomalt, sucralose, acesulfame potassium, analogs and derivatives thereof, and combinations thereof.

Caffeine: Caffeine is a xanthene alkaloid extracted from the seed of the coffee plant. It functions as a central nervous system stimulant. Caffeine is typically used to increase wakefulness, faster and clearer thought and to combat drowsiness. Caffeine is thus a naturally occurring cognitive enhancer (Simons et al 2011) and its long term use correlated with an increase in cognitive ability and memory in later life (Corley et al 2010), a reduced risk of cognitive decline and risk in midlife (Eskelinen et al 2009) and—given the heterogeneity of results inherent in epidemiologic studies—a lowered prevalence of dementia and AD (Eskelinen and Kivipelto 2010; Santos et al 2010 (a)). This benefit of caffeine is associated with an average daily consumption of 3 to 5 cups of coffee a day and is more likely to be found in women than in men (Santos et al 2010 (b); Arab et al 2011).

Caffeine inhibits adenosine (Simons et al 2011). Adenosine is found in all tissues, and in the central nervous system, suppresses neurotransmitter activity. By antagonizing adenosine, caffeine increases the activity of acetylcholine, epinephrine, dopamine, serotonin, norepinephrine and glutamate.

Caffeine also inhibits acetylcholinesterase (Karadishem et al 1991). Through this mechanism, caffeine has—in addition to its enhancing effect on ACh cognitive mediated function—been shown to counteract the cumulative burden of anticholinergic medications commonly used by the elderly (Nebes et al 2011).

### Brain Health Protection:

Brain Derived Neurotrophic Factor (BDNF): Long-term potentiation (LTP) modulates synaptic plasticity and is widely accepted as one of the initial events needed for memory encoding. LTP is impaired with aging and also in AD. BDNF regulates this synaptic plasticity in the adult brain (Diogenes et al 2011).

Caffeine increases hippocampal BDNF by modulating adenosine receptors and with chronic usage stimulates the conversion of proBDNF to mature BDNF (Sallaberry et al 2013); caffeine reverses the decrease in hippocampal BDNF noted in high fat fed animals. High fat diets, obesity and resulting type 2 diabetes, are recognized risk factors for AD (Moy and McNay 2012); caffeine freely crosses the blood-brain-barrier and in so doing promotes an increase in the length, branching and density of basal dendrites in hippocampal neurons (Vila-Luna et al 2012) and via an associated increase in BDNF synthesis, prevents the stress related reduction in synaptic long-term potentiation (LTP). The latter function is key to maintenance of long term memory (Alzoubiet al 2013).

Neurodegeneration: Caffeine, by modulating the antioxidant system in the brain prevents the age associated decline in cognitive function (Abreu et al 2011); in addition, caffeine shifts the balance between neurodegeneration and neuronal survival by stimulating pro-survival cascades and inhibition of proapoptotic pathways in the cerebral cortex (Zeitlin et al 2011).

Reducing the brain beta amyloid load: A number of animal experiments have demonstrated that caffeine decreases brain amyloid and improves the cognitive impairment associated with AD. Three main mechanisms were identified:

A decrease in the synthesis of beta amyloid from APP via the suppression of the beta-secretase and gamma-secretase expression. In one study involving AD in transgenic mice, the deposition of beta amyloid was reduced by 40% in the hipocampus and 46% in the entorhinal cortex (Arendash et al 2009). This results from caffeine itself—in a dose equivalent to 5 cups of coffee—and not the metabolites of caffeine (Arendash and Cao 2010). Although treatment with other beta and gamma-secretase inhibitors also reduced APP induced damage, caffeine was the most promising therapeutic intervention in both APP and tau-induced AD models (Stoppelkamp et al 2011).

Enhanced Brain Amyloid Clearance: caffeine up regulates the low density lipoprotein receptor related protein (LRP1 0 and the P-glycoprotein (P-gp) at the BBB. This is associated with an enhanced efflux of beta amyloid from the brain with an increase in the brain efflux index of 80% (Qosa et al 2012).

Facilitating CSF Production and Turnover: Compromised function of the choroid plexus and defective CSF production and turnover has been associated with a diminished clearance of beta amyloid and may be one mechanism implicated in the pathogenesis of late onset AD (Wostyn et al 2011). Caffeine increases CSF production together with an increased expression of Na+-K+-ATPase and an increased cerebral blood flow. This is a result of caffeine's inhibition of the A1 adenosine receptors in the choroid plexus and its negative regulation of Na+-K+ ATPase (Han et al 2009).

Increasing Insulin Sensitivity: Although the role of caffeine consumption on insulin action is still being debated, recent animal studies (Guarino et al 2012) and a large scale clinical study that included 954 multi-ethnic non-diabetic adults (Loopstra-Masters et al 2010) have confirmed that the chronic use of caffeine was associated with a decrease in age related insulin resistance via mechanisms involving beta cell function (enhanced bioconversion of proinsulin to insulin), by decreasing the production of non-esterified fatty acids (which increase peripheral insulin resistance) and by enhancing Glut 4 expression in skeletal muscle. The soy isoflavone estrogen genistein, up-regulates the expression of Glut 4 and decreases non-esterified fatty acid (NEFA) metabolism and peripheral concentrations.

### Relevant Clinical Outcomes:

Utilizing functional MRI (fMRI) testing, caffeine was shown to have a modulating effect on the brain regions—medial frontopolar and anterior cingulated cortex—associated with attention and executive functions (Koppelstaetter et al 2010).

Caffeine plus glucose: a double blind randomized study indicated that there is synergistic effect on sustained attention and verbal memory, when 75 mg of caffeine was combined with 75 g glucose (Adan and Serra-Grabulosa 2010).

Glucose energy drinks (Red Bull) combined with caffeine, have shown improvements in reaction times and a decrease in mental fatigue (Howard and Marczinski 2010).

### Metabolism and Pharmacokinetics.

Caffeine is absorbed by the small intestine within 45 minutes of ingestion and is then distributed throughout all tissues of the body (Liguori et al 1997). Peak blood levels are reached within one hour, and subsequently eliminated via first order kinetics (Newton et al 1981; Lelo et al 1986). The half life of caffeine—the time taken to eliminate one half of the total amount of caffeine—is about 4 to 6 hours (Newton et al; Lelo et al 1986).

Caffeine is metabolized by the liver's cytochrome P450 oxidase enzyme system into three metabolic and functional dimethylxanthines: Paraxanthine (84%); Theobromine (12%) and Theophylline (4%). Paraxanthine increases lipolysis and may lead to increased glycerol and free fatty acid blood levels; theobromine dilates blood vessels and increases urine volume; theophylline relaxes the smooth muscle of the bronchi, and in much higher concentrations is used to treat asthma.

Both caffeine and its major metabolite—paraxanthine—can be quantified and their systemic levels monitored in blood, plasma or serum (Klebanoff et al 1998).

### Natural Glucagon-Like Peptide-1 (GLP-1) Secretagogues

Sweeteners have been reported to enhance the release of GLP-1. GLP-1 has two main physiologic properties that are of relevance to the disclosed subject matter: stimulation of insulin secretion and enhancement of its peripheral tissue sensitivity; function as a neuroprotective peptide.

Glucagon-like peptide 1: The major source of GLP-1 is the ileal intestinal L cell that secretes GLP-1 as a gut hormone. It is the product of the proglucagon gene that is selectively cleaved into its biologically active form. GLP-1 and its receptor GLP-1R is also found in the pancreas (Hoist 2007). The GLP-1Rs have been identified throughout the CNS with binding sites present on glia and neuronal cells (Chowen et al 1999; Iwai et al 2006).

GLP-1 is an incretin and responds to nutrients in the lumen of the small intestine. The agents that stimulate its secretion—secretagogues—include nutrients such as carbohydrates, proteins and lipids. GLP-1 enhances the sensitivity of the pancreactic beta cells to glucose by increasing the expression of GLUT2. GLP-1 has a half life of only 2 minutes due to its rapid degradation by dipeptidyl peptidase IN/(Thum et al 2002). It is an important anti-hyperglycemic hormone as it induces both glucose-dependent insulin secretion and the suppression of glucagon secretion. GLP-1 does not stimulate insulin when the plasma glucose levels are in a normal fasting range (Koole et al 2013).

GLP-1 and GLP-1R regulate the differentiation of pancreatic progenitor cells and stimulate beta cell mass (Harkavyi and Whitton 2010; Yabe and Seimo 2011). The GLP-1R has a well accepted role as an anti-apoptotic agent by negating or reducing the pro-apoptotic actions of peroxides including exposure to reactive oxygen species (ROS), cytokines and fatty acids (Li et al 2003). In addition, GLP-1 increases the expression of anti-apoptotic genes such as Bcl2 and BclxI (Buteau et al 2004).

GLP-1 as a neuroprotective peptide: Evidence for the CNS effect of GLP-1 was originally based on its central control of satiety (Gunn et al 1996). As reviewed recently (Holscher 2012; Salcedo et al 2012) it has now been clearly established (in pre-clinical studies) that GLP-1 crosses the BBB and prevents neurodegeneration including preservation of memory function in AD and motor activity in PD. This is probably due to a number of processes: protection of synaptic activity and function; NGF-like induced neurogenesis (Perry et al 2002); reduced apoptosis; protection from oxidative stress; and possibly, the increased CNS effect of GLP-1 mediated insulin sensitivity. Insulin acts as a growth factor in the brain and supports neuronal repair, dendritic sprouting, synaptogenesis and negation of oxidative stress (Holscher 2012). Cell culture studies have shown that GLP-1R agonists protect neurons against beta amyloid and glutamate induced apoptotsis by modifying the processing of APP (Perry et al 2003) and by attenuating neuron atrophy following excitotoxic stimulation (Perry and Greig 2005).

Natural Stimulants of Endogenous GLP-1: The extremely short half life on GLP-1—2 minutes—was thought to preclude the clinical utility of natural GLP-1 secretagogues. A number of studies have investigated a variety of compounds that do have small intestine GLP-1 releasing activity. These include: olive leaves that secrete GLP-1 via a naturally occurring compound oleanic acid, and its activation of TGR5 receptors (Sato et al 2007); the amino acid glutamine that has been shown to stimulate GLP-1 in vitro and in vivo (Greenfield et al 2009); and chlorogenic acid, a biologically active dietary phenol found in coffee (Johnston et al 2003). This compound has an inhibitory effect on glucose absorption, has a direct action on beta cells and their response to an increase in plasma glucose and has anti-oxidant properties. Chlorogenic acid, counteracts the adverse impact of chronic free fatty acid overexposure on beta cell function in overweight insulin resistant subjects (McCarty 2005; Johnston et al 2003).

Macronutrients that slow gastric emptying and stimulate insulin secretion in advance of the main nutrient load, have also been shown to stimulate endogenous GLP-1. Thus, treatment with a tagatose/isomalt mixture did result in a delayed GLP-1 secretion due in part to the slowing of gastric emptying time with distal gut production of short chain fatty acids stimulating GLP-1 (Wu et al 2012).

Artificial sweeteners synergize with glucose to enhance GLP-1 release. This is mediated via stimulation of the sweet-taste receptors on the gut mucosa (Brown et al 2009). Absent of carbohydrates, sweeteners do not stimulate GLP-1 (Ma et al 2009). Slowing and prolonging the rate of absorption elicits postprandial responses characterized by smaller rises and slower falls of blood glucose and insulin, prolonged suppression of free fatty acids and a reduced glycaemic response after a subsequent meal (Wolver et al 1995; Liljeberg et al 1999).

Sucromalt (Xtend® Cargill) is an enzymatically modified blend of sucrose and corn syrup containing fructose, leucrose and glucose oligosaccharides. In a recent randomized crossover study, sucromalt increased the plasma levels of GLP-1 (sustained over a four hour time frame) to twice that of a test meal of high-fructose corn syrup (Grysman et al 2008). This was associated with a delayed rise in FFA's. These results—together with the lack in rise of the simultaneous measurement of breath H2—confirmed that the sucromalt was absorbed more slowly, principally from the colon. This is consistent with earlier studies demonstrating that slowly digested carbohydrates travel further down the intestine before being absorbed and stimulate a late rise in GLP-1 (Krause et al 1982; Juntunen et al 2003).

In another recent randomized cross over study, subjects showed significantly improved mental and physical energy (over 4 to 5 hours) after a solution of 75 g sucromalt compared to 75 g of glucose (Dammann et al 2012).

Glucose to stimulate Glucose Intestinal Polypeptide (GIP). Glucose stimulates the secretion of GIP. In experimental models, GIP induces the proliferation of hippocampal progenitor cells (Nyberg et al 2005) and also enhances the induction of long term potential (LTP) which is the physiologic cellular mechanism controlling learning. GIP protects the synapses from the detrimental effects of beta amyloid and thus on LTP (Gault et al 2008). Over-expression of GIP increases coordination and memory recognition (Ding et al 2006)

Since GIP is rapidly degraded by the enzyme DPP IV, the added glucose will be added to the BBBBB™ powder/beverage in a delayed and time released formulation.

### Formulations

Described herein are compositions formulated as pharmaceutical compositions and nutraceutical compositions for use in the treatment and prevention of diseases and conditions and for promoting brain health.

The compositions described herein optionally include one or more pharmaceutically acceptable carriers, diluents, or excipients. Pharmaceutically acceptable carrier, diluent, or excipient, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5th Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

The compositions described herein may be formulated into preparations in solid, semi-solid (e.g., gel), liquid or gaseous forms, such as tablets, capsules, powders, granules, ointments, solutions, suppositories, injections, inhalants and aerosols. As such, administration of the formulation may be achieved in various ways, including, but not limited to, oral, nasal, buccal (e.g. sub-lingual), rectal, topical (including both skin and mucosal surfaces, including airway surfaces), parenteral (e.g., subcutaneous, intramuscular, intradermal, intravenous and intrathecal), intraperitoneal, transdermal, intracheal, intravaginal, endocervical, intrathecal, intranasal, intravesicular, in or on the eye, in the ear canal, etc., administration. In certain embodiments, one or more pharmacological agents may be administered via a transdermal patch or film system.

In one embodiment, the compositions may be formulated for oral administration using pharmaceutically acceptable carriers well known in the art in dosages suitable for oral administration. Such carriers enable the pharmaceutical and nutraceutical formulations to be formulated in unit dosage forms as tablets, pills, powder, dragees, capsules, liquids, lozenges, gels, syrups, slurries, suspensions, etc., suitable for ingestion by the patient. Pharmaceutical and nutraceutical preparations for oral use may be obtained through combination of at least one pharmacological agent with a solid excipient, optionally grinding a resulting mixture, and processing the mixture of granules, after adding suitable additional compounds, if desired, to obtain tablets or dragee cores.

Accordingly, the formulations suitable for oral administration can be present in discrete units, such as capsules, cachets, lozenges, tablets, and the like, each containing a predetermined amount of the active components of the composition described herein; as a powder or granules; as a solution or a suspension in an aqueous or non-aqueous liquid; or as an oil-in-water or water-in-oil emulsion. Such formulations may be prepared by any suitable method of pharmacy which includes, but is not limited to, bringing into association the active pharmacological agent and a suitable carrier (which may contain one or more optional ingredients as noted above). For example, formulations for use can be prepared by uniformly and intimately admixing the active pharmacological agent(s) with a liquid or finely divided solid carrier, or both, and then, if necessary, shaping the resulting mixture. For example, a tablet may be prepared by compressing or molding a powder or granules containing the active pharmacological agent, optionally with one or more accessory ingredients. Compressed tablets can be prepared by compressing, in a suitable machine, in a free-flowing form, such as a powder or granules optionally mixed with a binder, lubricant, inert diluent, and/or surface active/dispersing agent(s). Molded tablets may be made by molding, in a suitable machine, the powdered pharmacological agent moistened with an inert liquid binder.

### Composition: Broad Based Balanced Bioactive Brain Blend™ (BBBBB™)

Huperzine A: As an extract of the plant Huperzia serrata of the locopodium alkaloid family (Lycopodiaceae) to contain from 1% to no less than 90% of pure Huperzine A, its synthetic equivalent (Wang et al 2007; Tudhope et al 2012; Koshiba et al 2009; Tun and Herzon 2012) or any derivative, analog, metabolite or combination thereof. This to include other acetylcholine esterase inhibitors: donepezil (Aricept®; Rivastigmine (Excelon®); Galantamine (Razadyne®) and Memantine (Namenda®) reference: Mayeux N. Eng. J Med 2012; 362:21942201. The dose of the hyperzine extract (and equivalence in all listed derivatives) to include 0.01 mg (10 mcg) to 150 mg (1500 mcg).

Phytoestrogens: The classes of phytoestrogens that may be used in the disclosed composition include at least one or more isoflavones, coumestans, lignans, or any combination thereof. The isoflavones which display estrogenic activity are preferred and include genistein, daidzein equol, biochanin A, formononetin, glycitein, the natural glycosides or metabolites of any of the isoflavones including their synthetic derivatives and in particular synthetic genistein (geniVida®—Metzner et al Arzneimitterforschung 2009: 59: 513).

The preferred phytoestrogens are extracted from soy; however, other sources may be used including clover, legumes, kudzu root, oilseeds, or any other phytoestrogen containing plants or chemically synthesized phytoestrogens (See U.S. Pat. No. 6,524,616; geniVida®).

The phytoestrogen—singly or with other similar natural estrogens in the combination product—to include a dose from 0.01 mg to about 1000 mg and in equivalent doses for all other derivatives natural or synthetic. Similarly, the phytoestrogen component of a combination estrogen product will include all commercially available phytoestrogens at all dosages.

The estrogen in the combination product can include—or be used together with—17-beta estradiol, estradiol valearate, ethinyl estradiol, estriol, conjugated equine estrogens (CEE-Premarin®), any active estrogenic ingredients of CEE, estrone, esterified estrogens, or any derivative, analog or metabolite of estrogen. The composition includes about 0.2 mg to about 2 mg estrogen. In one embodiment, the composition includes about 0.2 mg to about 1.0 mg of 17-beta estradiol; about 0.3 mg to about 0.625 mg of natural conjugated equine and/or synthetic conjugated estrogens; about 0.3 mg to about 0.9 mg of estradiol acetate; and about 5 mg to about 50 mcg of ethinyl estradiol.

Vitamin D3: The vitamin D3 component will be principally in the form of D3 cholecalciferol in a daily dose ranging between about 50iU to about 20,000 IU and/or equivalent doses of other vitamin D3 synthetic analogs or derivates, and adjusted according to the route of administration.

### Formulation: Broad Based Balanced Bioactive Brain Blend™ (BBBBB™)

The manufacture and dosing of the three principal components—Huperzine A, soy isoflavones and vitamin D—will be adjusted according to their pharmacokinetic absorptive and tissue distribution properties, so as to optimize their combined pharmacodynamic metabolic activity.

This will vary—but not be limited to—the development of combination blends as: a brain health supplement; a functional nutraceutical complement and/or as a medical nutraceutical complement. The clinical criteria determining the blend formulation, includes its use as either a supplement to support normal healthy aging; a functional nutraceutical complement for use in subjects with age related difficulties in memory, cognition and related CNS dysfunction including evidence of early mild cognitive impairment (MCI); or as a pharmaceutical complement for established MCI and evidence of early mild to moderate AD. Depending on the clinical situation and at the discretion of the supervising health care provider, all three formulations may be used adjunctive to treatments for disease specific conditions: MCI; AD; Type II diabetes: post menopausal HT; obesity; osteoporosis; osteopenia; hypertension; and other relevant cognition disabling conditions.

### Immediate and Extended Release Formulations

The compositions described herein can be formulated for immediate release, timed release, or extended release. The compositions can be administered once daily or twice daily. In one embodiment, for extended release (sequenced extended release), the composition may be administered once daily. In another embodiment, for immediate release, the composition may be administered twice daily.

Huperzine A: The definitive pK study for Huperzine A was published in 2008 (Wei et al 2008). Healthy subjects received 0.2 mg of pure Huperzine A orally. Plasma levels rose rapidly after administration peaking at about hour 1.2 to 1.3. The plasma levels declined rapidly over the next 24 hours and a terminal half life of approximately 6 hours determined. Although this form of immediate release Huperzine A, at appropriately adjusted doses for a given indication, may well serve the needs as a brain health supplement in otherwise healthy peri- and early postmenopausal women and/or as a complementary adjunct to subjects on specific disease related drugs, a controlled release formulation that would have a more prolonged therapeutic effect & provide once a day administration, is desirable.

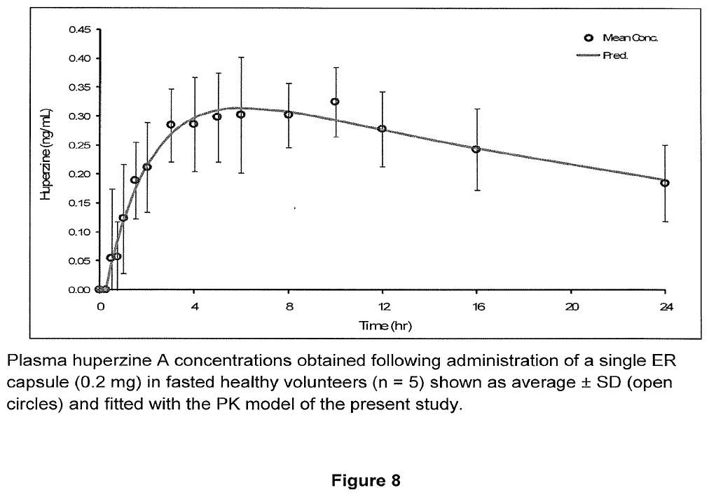

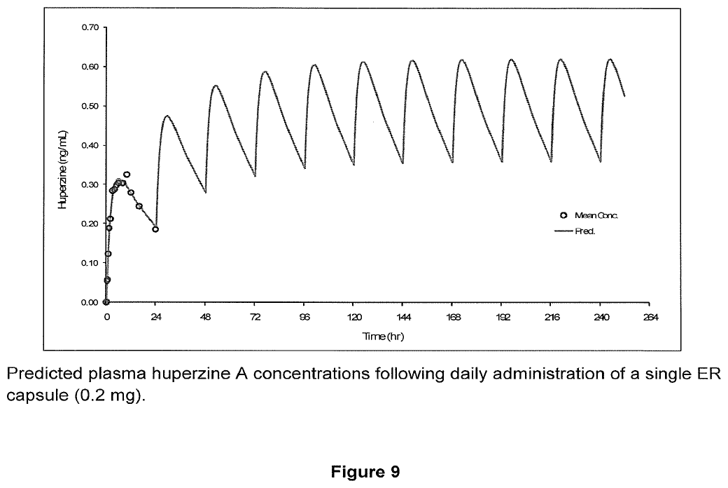

A novel, extended release Huperzine A formulation was studied under the principal aegis of the inventor, and was designed to compare the same doses (200 mcg) of an immediate release (IR) formulation of the Huperzine A herb with a specially manufactured extended release (ER) comparator. The result: The ER formulation raised plasma levels of Huperzine A in a much more gradual manner than the IR formulation and resulted in consistent plasma levels of Huperzine A in a range found to be both beneficial and safe in human subjects. (See FIGS. 8 and 9.)

Sequencing and Absorption with Specific Tissue Receptor Expression.

The vitamin D receptor (VDR) is up-regulated by 17-beta estradiol (Gilad et al 2005). In addition, estrogens increase tissue levels of activated vitamin D {1,25(OH)2D3} by increasing the vitamin D anabolic gene CYP27B1 and by decreasing CYP24 levels the Vitamin D catabolic gene (Lechner et al 2006). The same has been shown for genistein (Cross et al 2004).

Estrogen and vitamin D have complementing effects on sensory nerve pathways (Tague and Smith 2011). Although the same relationship has not as yet been proven for the CNS, this principle has been incorporated into the design for both of the planned functional nutraceutical and pharmaceutical complement products.

Estrogen and vitamin D are steroid hormones and have membrane and cytosolic receptors that result in expression abruptly in seconds to 60 minutes (membrane rapid response—RR) followed by a similar but slower cytosolic genomic response a few hours later (Norman 2006).

This ligand binding-/expression differential is incorporated into the design and manufacture of specific time release formulations that will allow estrogen absorption to precede that of Vitamin D.

### Manufacture of Broad Based Balanced Bioactive Brain Blend™ (BBBBB™)

All routes of administration for the above combinations can be used and include the following pill, capsules (hard and gel), tablet, powder, beverage, suspension, emulsion, syrup, solution, patch, gel, combinations thereof and the like.

For administrative purposes, the compositions can further include pharmaceutically acceptable, carriers, diluents, solubilizers, lubricants, binders, and the like or excipients thereof.

Formulation and Manufacture of Broad Based Balanced Bioactive Brain Blend™ (BBBBB™) with Additives

Caffeine: Caffeine is absorbed from the small intestine within 45 minutes of ingestion. Peak levels are reached within one hour, and subsequently eliminated via first order kinetics (Newton et al 1981; Lelo et al 1986). To optimize caffeine's complimenting & additive effect on brain health promotion and function to that of the BBBBB™, IR formulations of the caffeine ingredient will be added to the BBBBB™ brain health supplement; an ER formulation that will allow for a more sustained blood level of caffeine over a 10 to 12 hour time frame, will be added to the BBBBB™ functional nutraceutical and the BBBB™ medical nutraceutical. The doses of caffeine will range from 25 mg to 250 mg daily.

The BBBBB™ blend can include addition of chlorogenic acid, the biologically active dietary phenol found in coffee and green coffee, in IR and ER doses equivalent to that of caffeine.

The BBBBB™ with caffeine combination can be manufactured in capsules (hard and gel); pills; tablet; powder; beverage; suspension; emulsion; syrup; solution; patch; gel, combinations thereof and the like. The compositions may include pharmaceutically acceptable carriers, diluent, solubilizers, lubricants, binders, combinations thereof and the like.

Natural Sweeteners (NS). The natural sweeteners—for the reasons noted previously—are important ingredients with important GLP-1 stimulating activity and well documented positive CNS effects, complementary to the actions of the BBBBB™ ingredients and that of caffeine. The best NS example is that of sucromalt, a Cargill developed product (Xtend®) and a constituent of Abbot's Glucerna®. This is for the BBBBB™ Beverage, which in addition, is formulated to provide adequate and sustained amounts of glucose for brain energy.

Glucose: The addition in beverages and powder mixes of 75 G (range 25 to 200 G) of glucose for nocturnal brain energy to stimulate glucose-dependent insulinotropic polypeptide (GIP). Together with GLP-1, GIP is a physiologic incretin that is stimulated by enteroendocrine K-cells in the pancreas, adipose tissue, small intestine, bone and brain. GIP stimulates potent glucose dependent insulin and may have an important role in modulation of brain function and insulin resistance (Irwin et al 2010). GIP receptors have been identified in several areas of the brain—including the hippocampus and amygdala (Nyberg et al 2007)—as well as the GIP gene and GIP protein expression (Nyberg et al 2005).

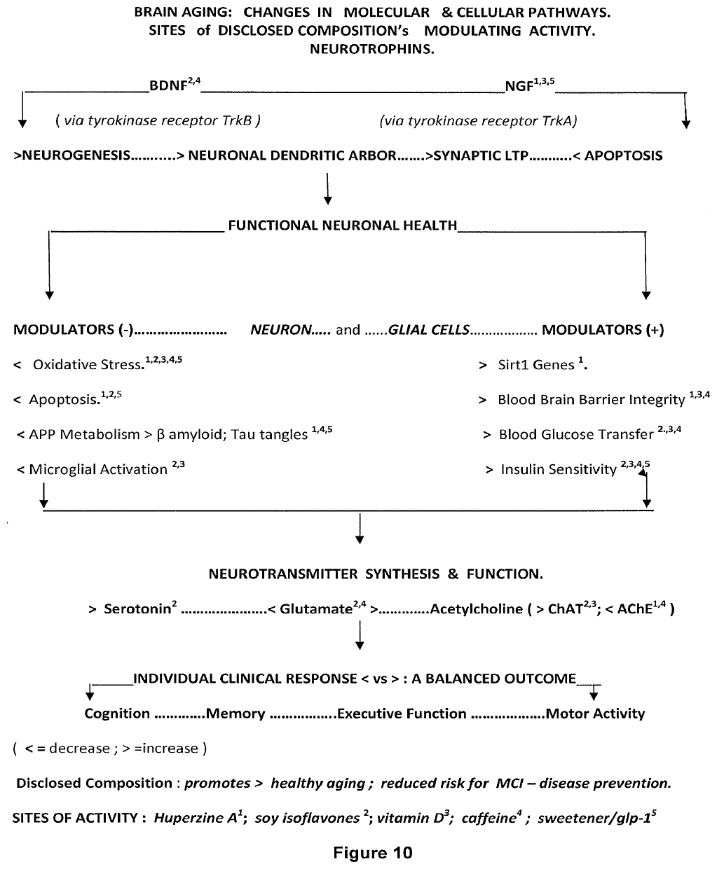

Pharmacologic Rationale: The rationale for developing the BBBBB™ with and without additives, is to provide a range of products that can promote the health of the brain as it ages and to modify metabolic abnormalities associated with the aging process per se, which would otherwise lead to the development of severe cognitive dysfunction and disease including MCI and AD. (See FIG. 10.)

The “art” of our BBBBB™ alone and BBBBB™ PLUS additive combination products is to combine the proven pharmacologic actions of each ingredient in order to promote healthy brain aging and/or to modulate abnormal molecular pathways associated with an increased risk of cognitive dysfunction. Some examples of the pharmacological “art” include the following “balanced” pharmacodynamic brain protective combinations:

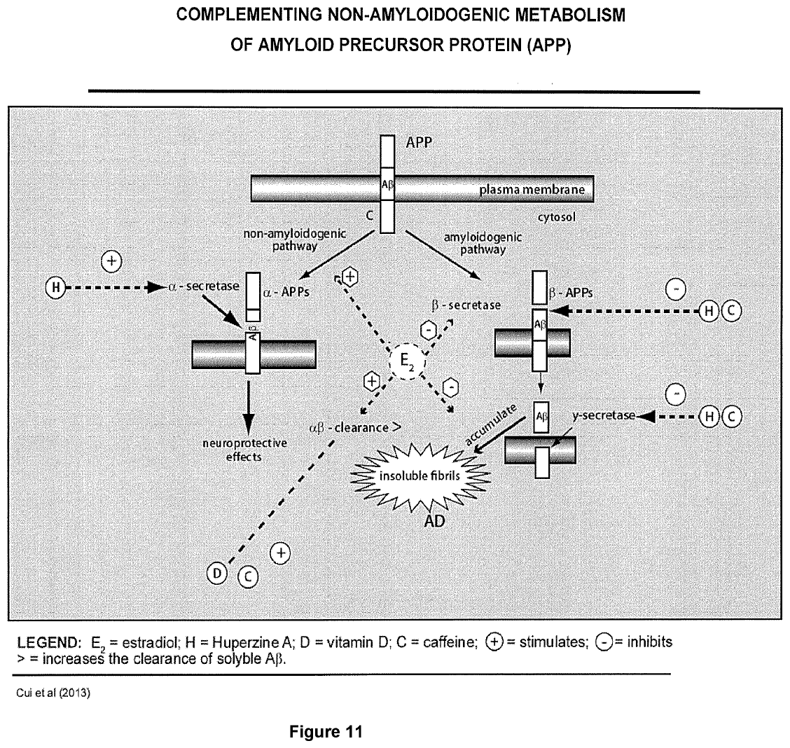

“Complementary”: Enhancing acetylcholine neurotransmission. Estrogen and vitamin D increase acetylcholine synthesis via increase choline acetyltransferase (ChAT) activity; Huperzine A and caffeine inhibits its breakdown by decreasing acetylcholinesterase (AChE).
a. Enhancing neurogenesis: Huperzine A, vitamin D, and GLP-1 increase nerve growth factor via the TrkA pathway; estrogen (genistein) and caffeine increase BDNF via the TrkB pathway.
b. Modulating APP metabolism: Huperzine A stimulates the alpha secretase and caffeine inhibits the beta and gamma secretase APP pathways with a resultant decrease in amyloid beta and tau protein accumulation. (See FIG. 11)
c. Beta amyloid clearance: 17-beta estradiol (E2), vitamin D, and caffeine increase beta amyloid clearance.

“Additive”: ingredients with the same biologic effect. Huperzine A, genistein, vitamin D all inhibit oxidative stress and hence enhance neuronal apoptosis.

“Synergistic”: first ingredient up-regulates the receptors for a second ingredient thus enhancing the biologic activity of the latter. Estrogens, natural and synthetic, and phytoestrogens up-regulate the vitamin D receptor.

Clinical Practice: Positive clinical outcomes of the methods provided herein are predicated on:

Timing: Subjects age and stage of cognitive disease if present. The presence of viable neurons responsive to the pharmacologic action of the various ingredients are important.

Continuence: Long term supplementation: the progression of the neuronal changes in both healthy and unhealthy aging, is gradual and requires long term continuance of the indicated health supplements and/or the functional & medical nutraceutical complements. Benefit is lost when treatment is stopped.

Biomarker measurement: Measurement of biomarkers indicative of ingredient absorption and efficacy to provide supportive evidence of healthy brain aging in otherwise asymptomatic women and so encourage long term continuance; in women with cognitive and memory dysfunction, biomarker testing for adjustment of the dosage of prescribed product depending on the clinical symptomatic response. The increase in the level of biomarkers in a subject is compared with the level of the same biomarkers in a healthy subject.

### Molecular Biology of Brain Aging and Pharmacodynamics of the Composition

Product Formulation: Provided herein is a range of compositions and formulations that will promote the health of the brain as it ages in otherwise healthy asymptomatic subjects; to modulate the multiple metabolic pathways associated with aging resulting in reduced cognition, memory and loss of executive function; and to prevent and/or delay the progression of cellular changes associated with severe cognitive dysfunction and disease including mild cognitive impairment (MCI) and Alzheimer's Disease. The composition's will be respectively formulated as a brain supplement; a functional brain nutraceutical and a brain medical food/nutraceutical. Each of these products will be used for both single preventive management and as an adjunctive to specific drug therapy for conditions associated with an increased risk of age related cognitive decline including established MCI and AD.

Brain Health Supplement: the preferred daily dose ranges of the three ingredients will include but will not be necessarily limited to: soy iosflavones 110 mg; Huperzine A 50 mcg; Vitamin D 800 iu.

Functional Brain Nutraceutical: this product will include three formulations to allow for a subject's individualized needs and response to a given prescribed dosage. The preferred daily dose ranges of the four ingredients will include but not be limited to the following combinations:

Soy isoflavones 110 mg; Vitamin D 1200 iu; Huperzine A 175 mcg; Caffeine 75 mg.

Soy isoflavones 110 mg; Vitamin D 1200 iu; Huperzine A 275 mcg; Caffeine 75 mg.

Soy isflavones 110 mg; Vitamin D 1200 iu; Huperzine A 375 mcg; caffeine 75 mg.

Brain Pharmaceutical Composition: This product includes but is not limited to the preferred daily dose ranges noted under “functional brain nutraceutical” and also the following: synthetic genistein 30 mg; synthetic huperzine in dose equivalent to Huperzine A 175, 275, and 375 mcg; vitamin D 1200 iu and caffeine 75 mg.

Product and Subject selection: successful treatment outcomes depend on matching the composition to the clinical needs of the subject and to monitor/adjust the treatment over time, depending on the clinical response. In addition to maintenance and/or symptomatic improvement in cognition, memory and executive function this requires baseline physical assessments and the measurement of biomarkers relevant to the subjects general and brain health status plus risk factors for cognitive dysfunction, including but not limited to MCI and AD.

### Subject Evaluation:

### No Known Risk Factors

General physical examination to include: weight and body mass index (<27 kg/m2); waist/hip ratio measurement (<0.8); blood pressure (<130/75 mmHg) and the following blood tests (normative values in parenthesis).

Total cholesterol (120-200 mg/dL); HDL cholesterol (>40 mg/dL); Triglycerides (<150 mg/dL); LDL cholesterol (<130 mg/dL); free fatty acids (0.07-0.88 mmol/L); Fasting blood glucose (70-110 mg/dl); Hemoglobin A1C % (<6); C-reactive protein (<5 mgL); 25-OH Vitamin D (30-100 ng/ml); Liver Function test panel.

### Known Risk Factors

Obesity and Type II Diabetes: above plus fasting insulin (4-27 ulU/ml); oral glucose tolerance test;

Hypercholesterolemia: above plus 27-hydroxycholesterol (Ghribi 2008) and Apolipoprotein panel: Apo A, Apo H and Apo J (Song et al 2012).

Osteopenia and Osteoporosis: above plus bone density measurement of the hip and lumbar spine with DEXA testing utilizing standard definitions (t score for osteopenia 1 to 2 standard deviations below young normal with no clinical radiologic fractures deformation of lumbar/thoracic vertebrae; osteoporosis: Bone mineral density (BMD) t score 3 or more standard deviations below young normal with or without evidence of vertebral deformation/fracture). Also, selective use of biomarkers of excess bone turnover: urinary/serum n-telopeptide levels; excess urinary calcium excretion (ca/creatinine ratio >16).

Hypertension: above plus hypertension (blood pressure greater than 140/90) or progressive increasing blood pressure.

Inflammatory Markers: Given the significant role of inflammation in the pathogenesis of AD, the following biomarkers are included (but are not limited) to the following cytokines, chemokines, growth factors, complement and adhesion molecules: they can be selectively used as both risk factors, measures of progression of disease and response to treatment: IL-1; IL-2; IL-4; IL-8; IL-10; IL-13; TNF-alpha; osteopontin and two anti-inflammatory markers: G-CSF, Fetuin-A and combinations thereof.

Symptomatic with/without Family History of MCI & AD

Cognitive tests: Clinical dementia rating (CDR: 0 equals normal; 0.5 very mild impairment; 1 mild impairment); Mini-Mental State Examination (MMSE: 0 equals severe impairment vs 30 no impairment); Wechsler Memory Scale-Revised (0 equals no recall to 25 complete recall) (Bateman et al 2012).

Blood tests: APOE genotype (Apoe4 allele); sirtuin 1 (alpha secretase; beta and gamma secretase); proteomics biomarkers assay of AD autoantibody biomarkers (Nagele et al 2011; Shi et al 2009) and other related and recognized blood, urine and CSF biomarkers of risk for MCI and AD.

Monitoring Treatment: Dosage and Efficacy.

Early brain aging is asymptomatic with multiple molecular pathways regulating neuronal health and function. The metabolic heterogeneity of individual subjects adds an additional variable that will determine whether clinically effective concentrations of the composition's constituents are absorbed. Only measurement of relevant biomarkers can confirm that adequate dosing has been achieved and in addition, allow for the adjustment of the composition disclosed herein over time if needed. The goal is for the tested components and biomarkers to reach the optimal bioactive level after administration of the composition. The optimal level indicates that the subject is being effectively treated for the disease or condition or that the composition is effective in promoting and maintaining the health of the brain of the subject. Tests include, but are not limited, to the following:

Measurements of Tested Components: Assays are performed to measure the levels of various components in the subject. Plasma assays of Huperzine A (to be within the range of 0.3 to 1.5 ng/ml); total genistein (to be within the range of of 3.5 to 18 microM—Takimoto et al 2003); 30 mg of synthetic genistein (to be within the range of 400 to 500 ng/ml-Metzner et al 2009); 25-OH vitamin D (to be within the range of 30 to 110 ng/ml); caffeine (to be within the range 2 to 10 mg/L). The suggested test time intervals: three months after treatment; 6 months later and then annually.

Brain Health & Function Biomarkers: The following assays for measuring biomarkers were performed, before treatment commences, at 3 months, 9 months and then annually. The measured neurotrophic factors serve as surrogate biomarkers of neurogenesis, the balance between acetylcholine synthesis and catabolism and the metabolism of Amyloid Precursor Protein (APP) and the Wnt/beta catenin pathway.

Brain Derived Neurotrophic Factor (BDNF) Eliza Immunoassay: (to be within the range of 0.066 to 16 ng/ml); Nerve Growth Factor Immunoassay (NGF): (to be within the range of 3.9 to 250 pg/ml); Acetylcholinesterase activity (to be within the range of 10 to 600 U/L); Acetylcholine (Quantitative Colormetric assay: to be within the range of 10 to 200 micoM; fluormetric assay: to be within the range of 0.4 to 10 microM acetylcholine); assays measuring the expression of alpha secretase and beta/gamma secretase enzyme activity; titers of AD autoantibodies using proteonomic and related assay technology before and after treatment.

### Microencapsulation: A Technique for Controlled Drug Delivery.

The compositions described herein can be in a microencapsulated formulation and administered once per day for sequenced extended release. Microencapsulation is a process by which small droplets or particles of liquid or solid material are coated with a continuous film of polymeric material. The principle reasons for microencapsulation is to provide for a sustained or prolonged rate of drug release and to alter the site of absorption. This can be accurately controlled over a period of hours or even days and designed for pre-programmed drug release profiles in order to meet the therapeutic needs of the patient. Microencapsulation technology is particularly suited to orally controlled release drug formulation systems (Singh et al 2010; Bansode et al 2010), especially when multiple doses are required.

Given the varying pharmacokinetic profiles of the constituents of the disclosed composition (see individual pharmacokinetics in the text), specifically designed immediate and extended release combinations will be formulated to optimize local tissue bioactivity and function

Examples include an ER form of Huperzine A to allow for 24 hour tissue availability; slow release of caffeine over 10 to 12 hours; sequencing of early genistein absorption with slightly delayed vitamin D absorption to allow for the estrogen induced up-regulation and expression of the VDR to enhance the latter's in situ activity.

Comparison of Immediate Release (IR) with an Extended Release (ER) Formulation of Huperzine A.

This pK study was performed at Cetero Laboratories (St. Louis, Mo.) under the aegis of CogniFem LLC and Osmopharm Capsules USA. IR and matching ER formulated capsules containing 200 mcg of the Huperzine A herb were prepared by Osmopharm USA to meet specified clinical requirements.

### Huperzine A: Duration and Dose.

The definitive pK study for Huperzine A was published in 2008 (Li et al). Healthy subjects received 0.2 mg of pure Huperzine A orally. Plasma levels rose rapidly after administration peaking at about hour 1.2 to 1.3. The plasma levels declined rapidly over the next 24 hours and a terminal half life of approximately 6 hours was determined. Based on this data, an extended release product would be required for once daily administration, in order to meet optimal brain tissue concentrations.

Over the major part of the day plasma levels ranged from 0.3 to about 1.0 ng/ml where 0.6 ng/ml was determined to be the optimal level (Li et al 2008). Values above this level produce unnecessary exposure of the tissues to brief high levels of Huperzine A and increased loss due to excretion. Doses of 0.15 mg pure Huperzine A twice daily was shown to effective for the treatment of mild cognitive impairment, a potential pre-condition to AD (Li et al 2008).

Pharmacokinetics: Molecular Pathway Counter Balancing Thus Formulation.

(a) Huperzine A has short half life: thus formulation in twice daily dosage or as extended release.

(b) Genistein: up regulates estrogen receptor (mainly ER beta) and <ER alpha, and acts as a SERM. Can therefore be used together with estradiol.

(c) Ingredients have complementing pharmacologic actions: eg Huperzine A & vitamin D increases the blood level of NGF and soy and caffeine increases the blood level of BDNF.

(d) Ingredients have synergistic activity: genistein increases the tissue concentration of vitamin D receptor and promotes its in situ synthesis while decreasing its catabolism: hence sequencing the absorption of each ingredient.

(e) Natural product extracts vs synthetic active component: vitamin D, derivatives and analogs thereof and Vitamin D receptor modulators. In some embodiments, the natural product or extracts are used to make nutraceuticals and synthetic active components are used in pharmaceutical compositions.

As an example, the method of using the disclosed compositions is as follows.

(a) Gender specific: as an example, a female subject.

(b) Timing and thus dosage of nutraceutical and pharmaceutical compositions: lower dosage is administered during early “critical window” for healthy brain aging vs higher dosage is administered for cognitively impaired and with MCI/AD. The disclosure compositions are formulated for nutraceutical and pharmaceutical use. In one embodiment, the “critical window” is within 10 years of menopause of a female subject.

(c) Primary therapy or adjunctive use with disease specific therapies in women at greater risk of MCI/AD.

(d) Risk factors: type II diabetes; metabolic syndrome; obesity; osteopenia/osteoporosis; hypertension; and cardiovascular disease;

(e) Other conditions associated with neuronal damage: post concussion; PTSD; stroke; Huntingdon's disease; schizophrenia.

(f) Biomarkers are used to determine and measure risk factors, absorption of ingredients and biomarkers of brain efficacy. The level of biomarkers can be used to adjust dosages if needed and to aid with long term compliance in asymptomatic women.

### Results:

Five volunteer subjects were tested in a blinded cross over study with both the IR and ER formulated capsules, all containing 200 mcg of the Huperzine A herb. As expected, the plasma levels following the immediate release formulation rose rapidly after administration, peaking at about 1.4 hours. This was followed by a rapid decline over the next 24 hours. These data are similar to the results obtained by Wei et al, using the pure huperzine alkaloid. The ER formulation was absorbed more slowly and a smoother peak was obtained by 5.4 hours. The levels then declined slowly for the next 20 hours giving a blood half life that was significantly greater than was observed after the IR form.

Using a standard model {Phoenix™ WinNonlin 6.0 (Pharsight Corp, St. Louis, Mo.)} for simulating “steady state” plasma levels following multiple doses and using data obtained in the single dose study described above, it was demonstrated that within 5 days a steady state level was reached with an estimated Cmax of 0.62 ng/ml and a Cmin of 0.36 ng/ml. These values were identical to those observed in the Chinese studies where positive effects on mild cognitive loss had been observed.

### Conclusions:

In comparison with the IR formulation, the ER formulation Huperzine A was absorbed over a longer period of time with a resulting increase in the plasma half life. The initial gradual rise in plasma levels were then maintained at a steady level over an extended period.

Unlike the IR formulation, absorption from the ER formulation did not cause a spike in the plasma levels of Huperzine A. Less material is therefore lost due to early excretion following the administration of the ER formulation.

The ER formulation generated more consistent absorption of Huperzine A between subjects than the absorption observed following the IR formulation.

Steady-state simulations using an accepted computer model predict that by the fifth dose a steady state will be reached with plasma levels fluctuating over a narrow range of blood levels consistent with beneficial effects on memory dysfunction.

### Methods of Use

The compositions described herein can be formulated for use in the promotion or maintaining a healthy brain. Various pathways and factors are involved in maintaining a healthy brain or for the prevention or treatment of diseases or conditions associated with the brain health.

The compositions can be formulated for administering to subjects in need of treatment or in need thereof such as to increase level of neurotrophins, improve neuronal health, and promote neurogenesis.

The compositions described herein includes its use as either a supplement to support normal healthy aging; a functional nutraceutical complement for use in subjects with age related difficulties in memory, cognition and related CNS dysfunction including evidence of early mild cognitive impairment (MCI); or as a medical nutraceutical complement for established MCI and evidence of early mild to moderate AD. Depending on the clinical situation and at the discretion of the supervising health care provider, all three formulations may be used adjunctive to treatments for disease specific conditions: MCI; AD; Type II diabetes: post menopausal HT; obesity, osteopenia and/or osteoporosis; cardiovascular disease; and hypertension; and other relevant cognition disabling conditions. The composition can be used alone or as adjunctive therapy with other drugs.

The compositions can be formulated in the form of blends of a nutraceutical and/or pharmaceutical combination for the treatment of all cognitive/memory dysfunction resulting from and/or associated with Parkinson's Disease, Huntingdon's Disease; Stroke; Post Traumatic Concussion; Post Traumatic Stress Disorder, Schizophrenia. The composition can be used alone or as adjunctive therapy with disease specific drugs.

### Neurotrophins and Neuronal Health

Linking the clinically observed age related changes in cognition, memory and executive/motor function that occur in “healthy” aging with that associated with “unhealthy” aging (benign senescent forgetfulness) and as a pre-condition to and risk for mild cognitive impairment and its later progression to Alzheimer's Disease.

Linking these observations with validated molecular brain research—in animal experiments and observational & noninvasive human studies—that establishes and defines the multiple interconnected pathways responsible for the clinically noted changes in cognition, memory and executive/motor function associated with “healthy” and “unhealthy” aging.

Linking the known molecular function(s) of botanicals and other natural compounds and their physiologic/pharmacologic effect on the established neurocognitive pathways associated with the altered cognition, memory and executive function, in both “healthy” and “unhealthy” aging Linking the multiple biologically altered molecular pathways associated with “healthy” and “unhealthy” aging, and combinations of botanicals and other natural compounds, that have complementary, additive and or synergistic effects on brain function and health. (See FIG. 10.)

Linking the pharmacokinetics of the botanical and natural compounds into blends—with or without additional additives—in order to optimize their combined brain cellular function.

Linking the utility of adding clinically proven bioactive combination botanical products with complimenting molecular activity, to that of disease specific conditions associated with an increased risk of cognitive and other brain dysfunction and their treatment: Mild cognitive impairment and early stages of Alzheimer's Disease; post menopausal hormonal therapy; type II diabetes; obesity; osteoporosis; osteopenia; hypertension; chronic use of anticholinergic preparations, anti-depressant SSRI treatment (Deltheil et al 2008).

Brain-Derived Neurotrophic Factor (BDNF): Brain derived neurotrophic factor is a neurotrophin that regulates a variety of neural functions including selection of neural progenitor cells; increases the number and growth of hippocampal neuronal dendritic spines and their development into mature spines; enhances the production and survival of new neurons from stem cells in the hippocampus; matures and integrates new neurons into existing neuronal circuits.

Most importantly, BDNF increases synaptic number and enhances their plasticity and resistance to injury and disease. Together with its tyrokinase membrane receptor, full length TrkB, BDNF stimulates long term synaptic potentiation (LTP) an essential information storage function. Insulin-like growth factor interfaces with BDNF to enhance exercise induced synaptic plasticity (Ding et al)

BDNF also increases presynaptic glutamate release and induces neuronal proteins encoded for mitochondrial biogenesis, anti-oxidant and DNA repair enzymes (Rothman et al 2012; Gomez-Pinilla et al 2008; Yoshi and Constantine-Paton 2010).

Nerve Growth Factor. Nerve Growth factor is the first described member of the neurotrophin family. The mature form of NGF is derived from a precursor form (ProNGF) and in its activated form has both pro-apoptopic and neurotrophic properties. NGF binds to high affinity tyrosine kinase receptor TrkA.

Although NGF circulates throughout the body, its most important function with respect to the methods and compositions provided herein is its synthesis in the cerebral cortex and hippocampus and its promotion of the survival and outgrowth of CNS cholinergic neurons especially in the basal forebrain complex. As such, it is regarded as a potential protective factor for neurodegenerative disorders associated with these neurons (Aloe et al 2012).

Cholinergic pathways are associated with the regulation of NGF synthesis. Some acetylcholine esterase inhibitors (AChE) stimulate NGF like activity by potentiating the neuritogenic effect of NGF, and by increasing mRNA in primary astrocytes promote NGF-induced neuronal survival and function. NGF also protects responsive neurons from oxidative injury (Wang et al 2006).

### Neurotransmitters

Changes in neurotransmitters have an important role in modulating normal brain aging. The three main neurotransmitters relevant to the methods and compositions disclosed herein include serotonin, glutamate and most importantly acetylcholine. The composition described herein can be used to increase the levels of neurotransmitters, to inhibit the activity or level of cholinesterase, to increase the level of or activity of acetyl choline transferase, and to promote normal brain aging. Neurotransmitters include, but are not limited to, serotonin, glutamate, acetycholine and combinations thereof.

Serotonin: The levels of serotonin, which is principally associated with executive function, are age related and in addition, influence brain function by the signaling pathways with other age related molecules such as BDNF and IGF-I (Glorioso and Sibille 2011).

Glutamate: Glutamate is the main excitatory neurotransmitter in the central nervous system, with important roles in both neurotransmission and functional plasticity. Thus, glutamate facilitates the release of BDNF, is essential for LTP synaptic plasticity, neurogenesis and other activities associated with neuronal survival including changes in dendritic architecture (Glorioso and Sibille). Conversely even though the glutamate receptors decrease with age, excessive glutamate signaling in the aging brain may lead to neuronal death through excitotoxicity (Uranga et al). This is the result of an excessive Ca++ influx, with elevated intracellular concentrations of Ca++ and resulting cellular necrosis and apoptosis. Blockade of the glutamate receptors reduces the Ca++ influx and neuronal death due to glutamate exposure (Wang et al). Neuronal death by overstimulation of glutamate receptors is thought to be the final common pathway for a number of neurodegenerative diseases, including AD.

Acetylcholine (ACh): ACh is the neurotransmitter used by cholinergic neurons at the neurotransmitter junction and plays a key role in the brain's memory related circuit. ACh is synthesized from choline and acetyl coenzyme A by the enzyme choline acetyltransferase (ChAT). This requires the transport of choline into cells from the extracellular space and the activity of ChAT. The levels of acetylcholine and cholinergic activity decline in the aging brain (Uranga et al) and especially in patients with cognitive dysfunction, including Alzheimer's Disease (AD). The synthesis, and therefore the levels of Ach, is balanced by acetylcholinesterase inhibitors (AChE). Reduction in AChE activity is the basis for most currently available AD treatment and is associated with a variable increase in ACh. Although positive correlations have been noted between ACh levels and AChE activity in the frontal cortex and whole brain, the efficacy of AChE inhibitor treatment is ultimately dependent on the presence of sufficient cholinergic neurons capable of synthesizing acetylcholine. AChE treatment does not retard the loss of cholinergic neurons, and at best only provides temporary symptomatic improvement in cognition.

### Oxidative Stress, Cellular Damage, and Cellular Death

The compositions described herein can be used to reduce cellular damage and cell death. Excess oxidative stress results in cellular damage with subsequent tissue and organ dysfunction. Oxidative stress induces an increase in inflammatory signaling within the aging brain resulting in dysregulation of neurotransmitter function. This is due to the accumulation of nuclear and mitochondrial DNA damage and via an ROS-mediated mechanism leads to accelerated brain aging and neurodegeneration. Studies have shown that oxygen radicals also initiate the build up of amyloid and enhanced neurodegeneration. The severity of age related memory loss has been correlated with brain and plasma levels of antioxidants.

Cellular death (apoptosis) is a physiologic consequence of normal aging but is also a feature of various acute and chronic neurodegenerative diseases. Typical apoptotic changes occur when neuronal cells are exposed to stressors such as H2O2, Beta amyloid peptides and oxygen-glucose deprivation. The likelihood of neuronal apoptosis is in large measure regulated by the Bcl-2 family of proteins. High levels of Bcl-2 expression inhibit apoptosis. Conversely, an increase expression of P53 and Bax is associated with the initiation of apoptosis (Wang et al).

The sirtuin family of longevity genes have been identified as key brain aging modulators. Their effects have been noted in both neuronal and glial cells and are associated with a reduction in the accumulation of misfolded proteins, the response to stress and the prevention of inflammatory pathways in glial cells that lead to mitochondrial dysfunction and cell death. SIRT1 has been shown to be a key player in neurogenesis by activating the gene for BDNF and potentiating its transcription factor, as well as that of other CREB target genes in the brain. SIRT1 regulates glucose homeostasis, controls insulin sensitivity in skeletal muscle and energy expenditure in the brain (Dong 2012).

SIRT1 activates the alpha secretase pathway that directs the processing of the amyloid precursor protein away from the production of beta amyloid peptide, thereby reducing the risk of AD. Over expression of brain SIRT1 in mice has been shown to reduce the load of the beta amyloid protein aggregates characteristic of the extracellular amyloid plaques in AD. In separate studies, SIRT1 was shown to destabilized the tau protein and reduce intracellular tau tangles (Guarente 2011). A loss of SIRT1 is closely associated with the accumulation of beta amyloid and tau in the cerebral cortex of patients with AD (Julien et al 2009).

### Inflammation and Brain Health.

The compositions described herein can be used to reduce inflammation and promote brain health. Inflammation has a significant role in the pathogenesis of brain health including both MCI (Roberts et al 2009; Sun et al 2013) and AD (Leung et al 2013; Kim et al). A number of cytokines and chemokines have been identified as contributing to activation of the microglia leading to the formation of beta amyloid/microglial complexes that in the early stages of AD precedes subsequent tau related neurofibrillary pathology and neuronal death (Eikelenboom et al 1996; Griffin 2006; Ray et al 2007).

Elevation of a number of different plasma cytokines have been positively correlated with severity of disease and progression of disease as assessed by memory tests, and even neuroimaging studies (Leng et al 2013). Plasma cytokines communicate with the brain, and circulating levels of peripheral cytokines have been shown to reflect brain cytokine levels (Banks et al 2002). One route involves diffusion of cytokines from the blood to the brain through an impaired blood brain barrier (BBB), with active transport across the BBB (Banks et al 2002). Another involves cytokine activation of the endothelium signaling to macrophages in the brain (Perry 2004). Apart from inflicting cellular damage, certain cytokines may stimulate the GSK-3 beta and p38-MAPK kinase pathways and via the up-regulation of Dkk1 antagonist, decrease Wnt/beta catenin signaling. Disruption of the Wnt/beta ctenin pathway has been implicated in neurodevelopment and many neurologic diseases such as AD and schizophrenia. Over expression of GSK-3beta impairs neurogenesis (He and Shen 2009) and increases tau hyperphosphorylation in the hippocampus (Lucas et al 2001). Blocking interleukin-1 signaling, improves cognition, attenuates tau pathology and restores the Wnt/beta catenin function in an animal model (Kitazawa et al 2011).

There are two protective proteins: G-CSF (granulocyte colony stimulating factor) which suppresses the production or activity of pro-inflammatory cytokines (Sanchez-Ramos et al 2009) with reduced plasma levels found in patients with AD (Laske et al 2009). Fetuin A, an abundant plasma protein that is synthesized in the liver and in the context of cerebral ischemia has been shown to be anti-inflammatory. A recent study correlated plasma levels of fetuin-A and the pro-inflammatory cytokine TNF-alpha in subjects with early AD and age matched controls. The patients with AD had significantly lower levels of fetuin A and higher concentrations of TNF-alpha (Smith et al 2011).

In addition, higher plasma levels of plasma fetuin-A have been associated with better performance on tests of global cognitive and executive function, with a lower likelihood of decline in theses cognitive parameters in older adults (mean age 75) when followed for 4 years (Laughlin et al 2013).

Examples of pro-activating inflammatory markers include but are not limited to cytokines, chemokines, growth factors, complement and adhesion molecules: they can be selectively used as both risk factors, measures of progression of disease and response to treatment: IL-1; IL-2; IL-4; IL-8; IL-10; IL-13; TNF-alpha; osteopontin and two anti-inflammatory markers: G-CSF and Fetuin-A.

### Blood-Brain Barrier (BBB) and Brain Health

The compositions described herein can be used to promote and maintain blood-brain barrier (BBB). The BBB consists of a specialized endothelium of brain capillaries that protects the central nervous system by separating it from the systemic circulation. It serves as both a physical and metabolic barrier that protects the microenvironment of the brain and hence its functional activities. Disruption of the BBB leads to compromised synaptic and neuronal function. The integrity of the BBB is due to tight junctions between adjacent endothelial cells that consist of three highly specialized transmembrane proteins that exert their protective effect via the blockage of cell surface adenosine receptors, inhibition of cAMP phosphodiesterase activity and by modulationg the release of calcium from intracellular stores (Chen et al).

Altered BBB function is key to the processes leading to mild cognitive impairment (MCI) and AD, due in part to the accumulation of beta amyloid in the brain. This results from allowing an increased beta amyloid influx into the brain and an inadequate beta amyloid efflux from the brain. In addition, beta amyloid is synthesized in and around the BBB and in the brain microvasculature. The presence of beta amyloid adversely effects brain endothelial cell function. BBB dysfunction is one of the earliest pathologic events leading to AD.

Associated risk factors for disruption of the BBB include atherosclerosis, stroke, diabetes and proinflammatory and other neurotoxic factors such as reactive oxygen species (ROS). The result: a leaky BBB that allows peripheral inflammatory cells to infiltrate into the brain parenchyma with subsequent activation of astrocytes and microglia both of which have been implicated in the pathogenesis of AD (Chen et al).

### Regulation of Brain Glucose and Insulin Resistance

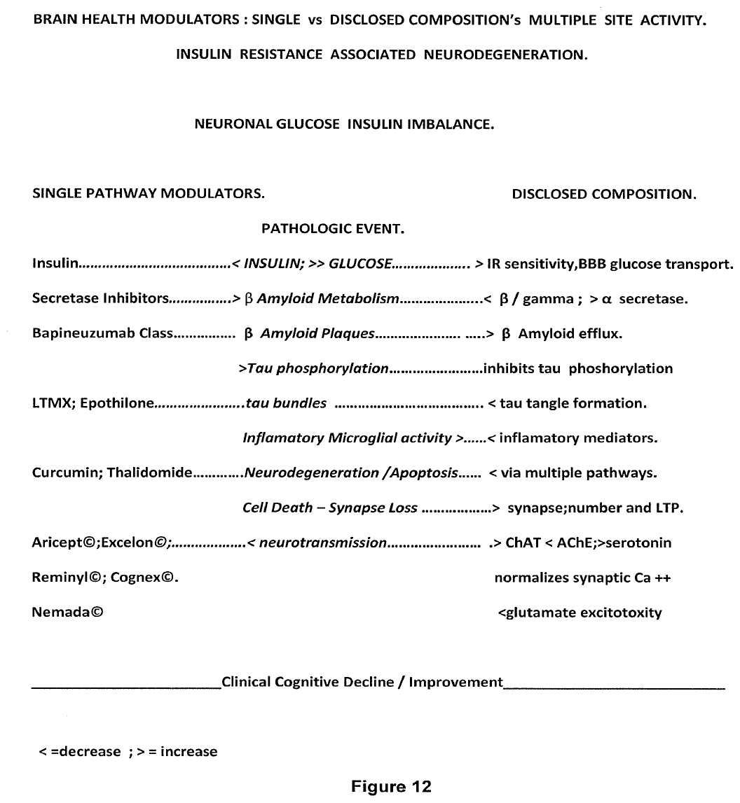

The compositions described herein can be used to regulate the supply of glucose and facilitate the transport of glucose across the BBB. Glucose is the essential nutrient for brain glucose metabolism and the energy needs of neurons. To meet ongoing mental demands and since the brain only maintains a 2 minute supply of glucose, two essential physiologic processes are needed: facilitation of glucose transport across the BBB; utilization of this glucose, and hence brain tissue insulin sensitivity. (See FIG. 12.)

Regulation of Glucose in the Brain: Three coupling steps are involved to initiate neuron activation and their need for glucose: release of glutamate from astrocytes signaling glucose metabolism and via this neurobarrier process stimulating the second stage; allowing for movement of glucose from plasma into the brain via the endothelial BBB cells glucose carrier protein GLUT1; with a final coupling step involving the relaxation of smooth muscles of the relevant arterioles, an increase in the blood vessel diameter and blood flow. This neurovascular and neurobarrier coupling is mediated through the metabolic activities of the neurons and astrocytes. The need for neuronal glucose is thus predicated by: brain activation, glucose transport, glucose support and the ability of the brain to utilize this energy source (Dorm ire 2009).

Brain glucose uptake and its metabolism is compromised in AD. This has been linked to a deficiency in the glucose transporters GLUT 1 and GLUT 3, and correlated with hyperphosphorylation of tau and to the density of neurofibrillary tangles (a hall mark of AD) in human brains (Liu et al 2008).

### Brain Insulin Resistance and Type Three Diabetes.

The compositions described herein can be used to prevent or inhibit insulin resistance in the central nervous system (CNS). Insulin is present in the adult CNS and is primarily derived from pancreatic beta cells. This insulin crosses the BBB via a carrier-mediated active process that is limited by the tight junctions between endothelial cells in the BBB. Chronic hyperinsulinemia down regulates insulin receptors (IR) at the BBB, thus impairing insulin transport into the brain. There is some animal data to suggest that insulin may be synthesized in the CNS following the detection of preproinsulin mRNA in the neurons (but not glial cells) of the hippocampus and prefrontal cortex.

Insulin is essential for normal CNS function. Once in the brain, insulin binds to IR that are widely distributed throughout the CNS, especially in the cerebral cortex and hippocampus. Both insulin and IGF-1 signaling pathways are involved in the regulation of brain metabolism, neuronal growth and differentiation and neuromodulation. Brain insulin increases neurite outgrowth, regenerates small myelinated fibers and by stimulating neuronal protein synthesis enhances synaptic activity and plasticity with resulting memory formation and storage. This effect is mediated via the expression of NMDA receptors, an increase in neuronal Ca++ influx and by reinforcing synaptic communication between neurons enhanced long-term potentiation (LTP).

Insulin and IGF-1 are also neuroprotective: brain neuronal apoptosis induced by oxidative stress is attenuated by insulin; IR/IGF-1 signaling mediates the gene transcription of anti-apoptotic factors such as increased Bcl-2 expression (Duarte et al 2012).

Insulin resistance: A number of studies have suggested that AD may represent the outcome of a metabolic disorder characterized by a deficit in brain glucose utilization. This is based on the demonstrated progressive decline in cerebral glucose utilization in subjects with AD. The abnormalities in insulin and insulin like growth factor (IGF) signaling and expression of insulin regulated genes results in insulin resistance and contributes the following AD like neurodegenerative changes an increase in the activity of kinases that hyperphosphorylate tau; the expression and accumulation of beta Amyloid Precursor Protein (beta APP) and its metabolism to its end product beta Amyloid; oxidative and endoplasmic reticulum stress; generation of ROS and reactive nitrogen species that damage RNA and DNA; mitochondrial dysfunction; activation of pro-inflammatory and pro-apoptopic (death) cascades (de la Monte 2012).

Although the “physiologic” insulin resistance associated with aging is the dominant risk factor for MCI and AD, insulin resistance associated with the following conditions also contribute to the neurodegenerative changes characteristic of AD: obesity; type two diabetes; and metabolic syndrome. Treatment with hypoglycemic or insulin sensitizing drugs may contribute to reducing the prevalence and/or severity of AD pathology and its clinical outcome (Luchsinger J A 2010).

At a functional level, insulin and IGF resistance down regulates the genes for the cholinergic activity that mediate neuronal plasticity and its concomitant effect on memory and cognition (de la Monte 2012).

### Wnt/Beta-Catenin Signaling and Regulation of its Dkk1 Antagonist.

### Wnt/Beta Catenin Signaling

The compositions described herein may be used to regulate the Wnt/Beta catenin pathway which is associated with the health of the brain and the development of AD. The compositions described herein can inhibit the activity of GSK-3 beta and increase the level of beta catenin, which inhibits the formation of amyloid plaques.

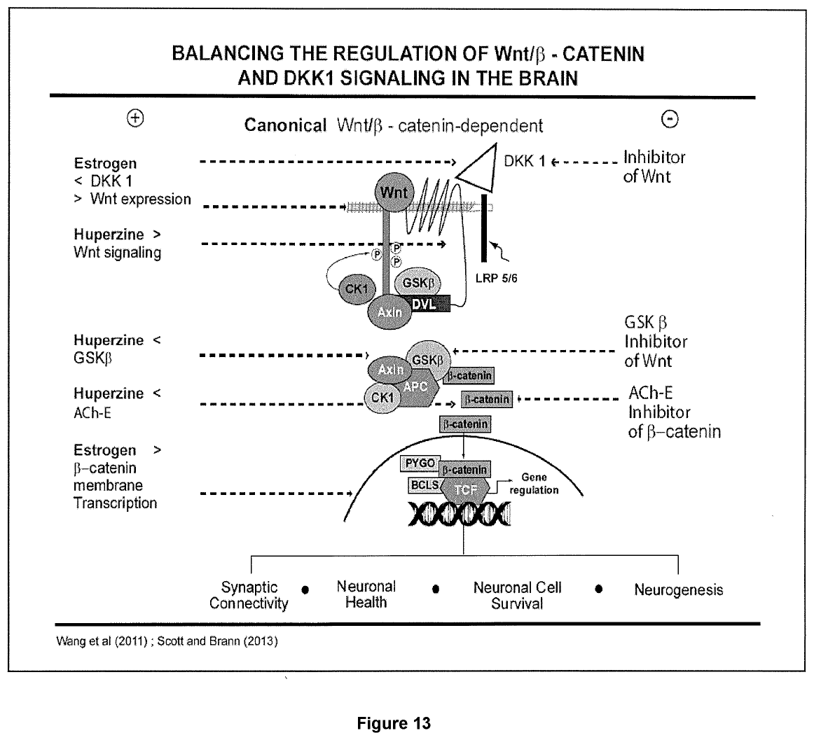

Wnt signaling is a transduction pathway governed by a variety of Wnt glycoproteins, which in addition to having a role in the development of the forebrain and the hippocampus (see later), are associated via alterations in its level and/or mutations with several pathologies including mood disorders, schizophrenia and Alzheimer's Disease (Inestrosa et al 2012; Maguschak and Ressler 2012; Kim et al 2013). (See FIG. 13.)

Although the Wnt proteins are traditionally classified as either canonical (eg Wnt-1 and Wnt 3a) or non-canonical (Wnt-4; Wnt-5 and Wnt-11), their activity at the cellular level depends in large measure to the presence of the frizzle (Fz) receptor on the receiving cell, and in the canonical pathway, a low density lipoprotein co-receptor (LRP 5/6). There are 19 Wnt ligands, 10 Frizzled receptors and 3 LRP co-receptors. There are over 120 target genes (Inestrosa et al 2012).

The classical canonical signaling pathway involves the binding of the extracellular Wnt ligand to the Fz receptor protein forming a cell surface complex with the related low density lipoprotein co-receptor (LRP5/6). This complex activates the phosphorylation of the cytoplasmic protein Dishevelled (Dvl), which in turn inactivates Glycogen-synthase-kinase-3beta (GSK-3 beta), thus preventing the degradation of beta catenin, which is then able to enter the nucleus of the receiving cell. Beta-catenin binds to a T-cell factor thus initiating the transcription of Wnt target genes. This results in a number of CNS functions including: development of the cerebral cortex and hippocampus; cell differentiation and adult neurogenesis (see later); cell proliferation, migration and differentiation; synaptic differentiation and glutamatergic functioning; inactivation of the inhibitor GSK-3beta; intracellular calcium dependent regulation thus strengthening the synaptic efficacy in developing neurons (Inestrosa et al 2012).

In short, the expression in the mature CNS of the Wnt ligand and its associated protein signaling pathways is central to its neuroprotection, pre and post synaptic plasticity (including an increase in its LTP—Chen et al 2006); axon guidance and dendritic morphogenesis (Zhou et al 2006); the up regulation of synaptic NMDA receptors (Cerpa et al 2011) and an increased efficacy of GABAergic synapses (Cuitino et al 2010). More recently, non-canonical Wnt/Ca signaling in the hippocampus has been shown to trigger nitric oxide production (NO) which in turn enhances NMDA trafficking and fine tuning of synaptic activity (Munoz et al 2012; Varela-Nallar et al 2011).

### Beta Amyloid and Wnt Signaling Pathways.

Wnt signaling protects against beta amyloid induced neuronal damage, and the activation of its pathway has been suggested as a therapeutic approach to the prevention of Alzheimer's Disease (Inestrosa et al 2012). There is a strong association between impaired Wnt signaling, beta amyloid induced neuronal damage and an increase in tau protein phosphorylation—all hallmarks of AD.

A number of the aforementioned components of the Wnt pathway are involved. For example, elevated levels of the Wnt inhibitory GSK-3 beta has been found in brains with established AD neurofibrillary changes and increased tau hyperphosphorylation with a concomitant decrease in the protective beta catenin (Pei et al 1999). Inhibition of GSK-3beta (with Lithium) protects rat neurons from beta amyloid damage (Inestrosa et al 2012) and up regulation of beta catenin prevents tau protein induced neuronal apoptosis (Li et al 2007). In short, exposure of hippocampal neurons (in rats) to beta amyloid results in the following three main Wnt related consequences: destabilization of the protective endogenous levels of beta-catenin; an increase in the inhibitory GSK-3beta activity; a decrease in Wnt target gene transcription.

### Wnt Signaling, Acetylcholinesterase (AChE), Alzheimer's Disease and Huperzine A.

Acetylcholinesterase is found in the neuritic plaques in the brain of AD sufferers (Guela and Mesulam 1995; Guillozet et al 1997) and enhances beta amyloid aggregation and plaque formation. The AChE-beta amyloid complexes may result in greater neuronal loss than just the beta-amyloid (Alvarez et al 1998; Reyes et al 2004). In addition, AChE-beta amyloid complexes have been shown to reduce the levels of cytoplasmic beta-catenin in cultured hippocampal neurons (Alvarez et al 2004) which is reversed by up regulation of the Wnt signaling by co-treatment with cascade activators (lithium) or antagonists (Alvarez et al 1999).

Huperzine A—in addition to its other neuroprotective effects (see before)—inhibits the activity of GSK-3 beta and increases the level of beta catenin, in both mouse brain and in cultured human neuroblastoma cells (Wang et al 2011). A recent study has shown that cross talk between the Wnt signaling system and PKC inhibits the activity of GSK-3beta and modulates the Wnt-catenin signaling thus regulating the phosphorylation of tau protein (and inhibiting neurofibrillary tangle formation) plus the processing of APP (Amyloid Precursor Protein) via the non-amyloidogenic pathway. The result: decreased amyloid plaque formation and neuronal apoptosis (Alvarez et al 2004; DeFerrari et al 2003; Wang et al 2011). These actions are complimentary to studies demonstrating Huperzine A's processing of APP via the non-amyloidogenic alpha-secretase pathway (Zhang et al 2004; Peng et al 2007; Wang et al 2011) and more recently, Huperzine A's inhibition of the amyloidogenic beta secretase pathway via its mediator, BACE1 (Wang et al 2011).

### Dickkopf-1 (Dkk-1) A Physiologic Wnt/Beta-Catenin Antagonist.

Memory impairment is associated with an age related decline in neurogenesis, with Dkk-1 a notable promoting factor via its inhibition of the canonical Wnt signaling pathway (Mac Donald et al 2009; Scott and Brann 2013). Long term estrogen deprivation leads to an elevation of Dkk-1 and dysregulation of Wnt/beta catenin signaling in the hippocampal neurons (Scott et al 2012). Conversely, loss of Dkk-1 in old age, restores hippocampal neurogenesis (Seib et al 2013).

Many studies have linked elevated levels of Dkk-1 to neurodegenerative diseases such as Alzheimer's Disease, Parkinson's disease, stroke and temporal lobe epilepsy (Scott and Brann 2013).

The cellular mechanism leading to Dkk-1 related neuronal dysfunction and death may result from an excess release of the excitatory neurotransmitter glutamate, with subsequent dose dependent (Cappuccio et al 2003) NMDA receptor activation and intracellular calcium overload (Zipfel et al 2000); the loss of protective Bcl-2, the induction of harmful Bax with hyperphosphorylation of microtubular tau protein (Scali et al 2006) following cerebral ischemic insults (Cappucio et al 2005; Scali et al 2006). The latter observation is complimented by the observation that patients with both ischemic stroke and confirmed coronary atherosclerotic plaques have elevated plasma levels of Dkk-1 compared with matched controls (Seifert-Heald et al 2011; Kimet al 2011). Dkk1 may therefore serve as a biomarker for these two diseases, and their association with an increased risk of cognitive dysfunction.

The accumulation of beta amyloid in cultured neuronal cells induces an over expression of Dkk-1 with subsequent hyperphosphorylation of tau protein and neuronal death (Caricasole et al 2004); higher levels of Dkk-1 expression is also found in post mortem human AD brain specimens (Caricasole et al 2004). Dkk-1 is up-regulated in the mouse model of fronto-temporal dementia and as in humans, was co-localized with neurons containing tau neurofibrillary tangles (Rosi et al 2010).

By blocking Wnt signaling, Dkk-1 prevents astrocyte associated neuroprotection (L'Episcopo et al 2011) and most importantly a decrease in the size of both the presynaptic and post synaptic terminals in mature neurons—without affecting cell viability—a feature typical of early memory loss due to “physiologic” brain aging related change (Purro et al 2012).

### Estrogen and the Balancing of Dkk-1 Expression and Wnt/Beta Signaling.

Estrogen (17-beta estradiol) promotes a favorable balance between Dkk-1 and Wnt signaling in the brain (Scott and Brann 2013) Mechanisms include suppression of post ischemic elevation of Dkk-1 following experimental global cerebral ischemia (Zhang et al 2008); by enhanced neuronal expression of Survivin, a Wnt target gene that inhibits neuronal apoptosis (Scott and Brann 2013); and inhibition of the GSK3 beta Wnt signaling antagonist via both ER alpha and ER beta (Varea et al 2010; Goodenough et al 2005) This results in stabilization of beta catenin and the prevention of tau hyper-phosphorylation (Zhang et al 2008).

Estrogen, also actives the neuroprotective PI3K-Akt kinase signaling pathway thus inhibiting/inactivating GSK3 beta with further stabilization and nuclear retention of beta catenin (Wandosellet al 2012).

In short, estrogen can modulate the Dkk-1 and Wnt/beta Catenin signaling by: suppression of the neurodenerative Wnt antagonist Dkk-1; up regulation of canonical Wnt/beta catenin signaling in neurons; promoting Wnt independent beta catenin transcription via a membrane ER initiated intracellular cascade involving the PI3K/Akt/GSK3 beta complex noted above.

### Complementing Ingredients of the Disclosed Composition on Wnt/Beta Catenin Signaling, and Linking to Clinical Biomarkers.

Huperzine A and estrogen have respective complementing effects on stimulating the Wnt/beta catenin signaling pathway and inhibiting its Dkk-1 antagonist. The bioactivity of this combination of the ingredients will promote the “balance” of this pathway and thus maintenance of synaptic connectivity, neuronal health, neurogenesis and neuronal cell survival.

Central to the methods provided herein, is the recognition of the need for early therapeutic intervention and so the need to define surrogate clinical biomarkers as measures of both brain health and the risk of later cognitive decline and dysfunction. One such biomarker is the measurement of plasma Dkk-1. Another can be measurement of bone density.

The biologic role of the Wnt/beta catenin signaling pathway has been demonstrated in a variety of other organ systems, with one central and common issue—the cytosolic concentration and mediation of the beta-catenin protein, with its subsequent organ specific target gene expression.

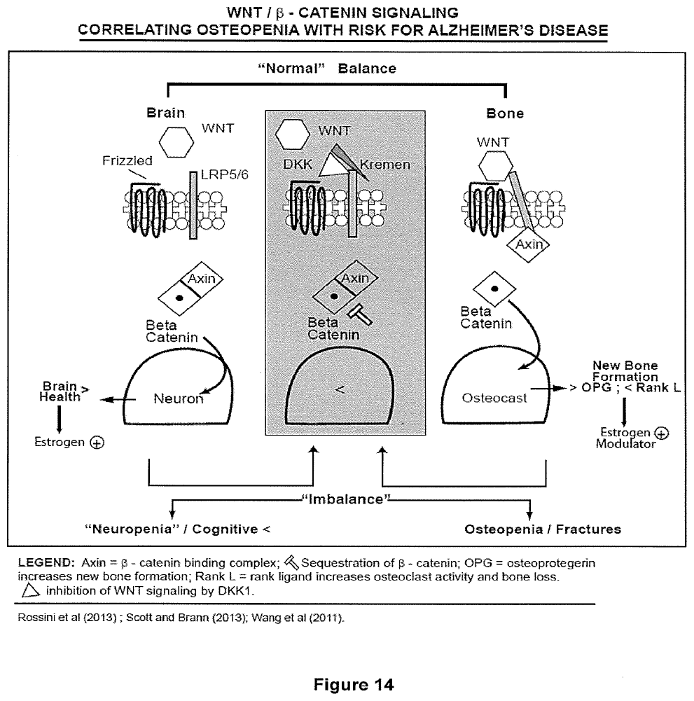

The Wnt/beta catenin pathway has a critical role in bone cells by enhancing osteoblastic activity and bone remodeling, mediated in part via the estrogen receptor (Rossini et al 2013). A number of clinical studies have noted the correlation between low bone density in patients with Alzheimer's Disease (Zhou et al 2011) and cognitive impairment in post menopausal women with low bone mass (Lee et al 2012) and hip fracture (Friedman et al 2010). The association with impaired cognitive performance has also been linked to vitamin D deficiency and lowered bone density in older African American women (Wilkins et al 2009). A phytoestrogen—diarylheptanoid (from the curuma comosa plant)—has been shown to activate the Wnt/beta catenin signaling pathway via an ER alpha Akt/GSK-3-beta complex (Bhukhai et al 2012) an action similar to that of estradiol. (See FIG. 14.)

Measurement of bone density (using DEXA technology) can thus be useful as a surrogate biomarker of Wnt signaling with reduced bone mass—osteopenia and or osteoporosis—indicative of an increased risk for later dementia.

### Adult Stem Cell Neurogenesis.

The compositions described herein can be used to promote adult stem cell neurogenesis. As an example, the compositions described herein can be used to increase the level of bone morphogenetic proteins in the brain, which is associated with the activation of neural stem cells. The compositions described herein can be used to activate the Wnt/beta-catenin signaling pathway and inhibit the Dkk1 and GSK3 beta activity.

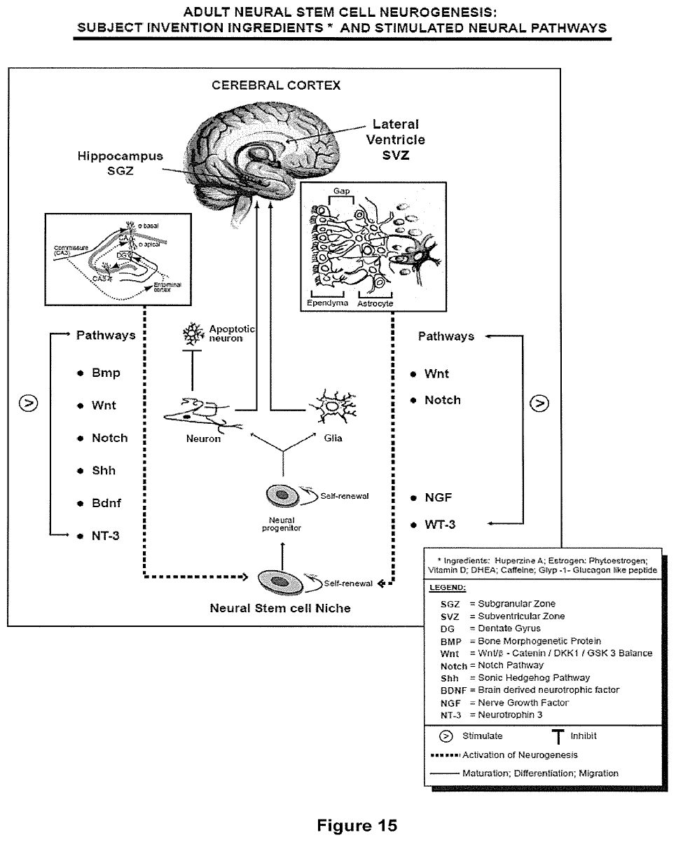

It has been clearly established that neurogenesis continues throughout life in the mammalian brain, including that of humans (Faigle and Song 2013; Encinas et al 2013; Eriksson et al 1998; Roy et al 2000; Wang et al 2011). This complex process takes place in just two regions of the mammalian brain: the subventricular zone (SVZ) of the lateral ventricles and the subgranular zone (SGZ) of the hippocampal dentate gyrus (DG). This complex and dynamic process is governed by a number of integrated factors that create a local “check and balance” microenvironment in the so-called “stem cell niche”. It is in this part of the brain where neural precursors—via cell to cell interaction—react to secreted factors and neurotransmitters resulting in differentiated glial cells and neurons, and some into hippocampal astrocytes (Song et al 2002). (See FIG. 15.)

A number of soluble extracellular factors have been identified that regulate stem cell signaling pathways: bone morphogenetic protein (Choe et al 2013-review); Wnt/beta catenin pathway (see before); Notch (Louvi et al 2006; Yoon and Gaiano 2005); sonic hedgehog (Traiffort et al 1998; Lai et al 2003); neurotrophins and neurotransmitters (see before).

Bone morphogenetic proteins (Bmp) constitute a sub group of the transforming growth factor-beta (TGF-beta) super family and are highly expressed in the adult nervous system (Bragdon et al 2011). They regulate a number of cell processes including cell survival, proliferation and differentiation (Harvey et al 2005; Liu and Niswander 2005). Bmp is derived from the menengial choroid plexus and regulates the stem cell niche in the DG via the Acvr1 receptor. This in turn regulates the expression of Lef1 in the DG stem cells, a key factor in Wnt signaling responsiveness (Choe et al 2013; Faigle and Somg 2013).

Bmp also plays a key role—together with its natural Noggin inhibitor—in activating neural stem cells (via Smad4—Colak et al 2008) to enter the cell cycle (Mira et al 2010). Changes in neurogenesis during the initial stages and progression of AD, has been associated with the modulation of new brain cell formation at neurogenic sites and subsequent hippocampal function. This is related in part to the balance between Bmp4 and its Noggin antagonist (Xu et al 2013).

Application: Estrogen up regulates Bmp in the brain and has been linked to both pituitary and hypothalamic function (Otani et al 2009). The expression of Bmp in hypothalamic neurons is via the rapid membrane associated ER. Membrane ER's have been observed in other brain regions, including the cortex, hippocampus and brain stem.

The surrogate low bone mass biomarker (osteopenia/osteoporosis) noted before is also associated with Bmp signaling: estrogen up-regulates Bmp4 and facilitates osteoblast differentiation (Matsumoto et al 2013), while genistein promotes osteogenic differentiation through Bmp/Smad signaling (Dai et al 2013) Thus, adding to the “connection” between brain health and bone health.

Wnt/beta-catenin pathway. The Wnt glycoprotein is highly expressed in the DG hilar cells and in cultured hippocampal astrocytes. Through its signaling pathway, Wnt mediates neuroblast proliferation and the neuronal differentiation of adult hippocampal progenitor cells (Lie et al 2005). The latter occurs via NeuroD1 transcriptional activation (Kuwabara et al 2009). The Wnt pathway, by stabilizing beta catenin and its cytoplasmic inclusion, activates other downstream transcription factors that prevent premature cell cycle exit and so neuronal differentiation (Mao et al 2009).

Over expression of Wnt subtypes have been shown to promote proliferation and neuronal differentiation of adult SVZ neuronal progenitor cells (Adachi et al 2007).

Application: Huperzine A activates Wnt/beta-catenin signaling (Wang et al 2001) while estrogen and phytoestrogens (respectively) inhibit its Dkk1 and GSK 3 beta antagonist activity (Scott and Brann 2013; Bhukhai et al 2012).

Notch pathway. The Notch pathway participates in many cellular processes in the developing nervous system including cell proliferation, differentiation and apoptosis (Louvi et al 2006; Yoon et al 2005). Notch is expressed in both the SVZ and the SGZ and regulates the NSC by controlling cell cycle exit, as well as maintenance and differentiation of adult neural stem cells (Breunig et al 2007; Imayoshi et al 2010). Notch has an important role in the dendritic arborization of immature neurons in the adult brain (Breunig et al 2007).

Application: A recently discovered selective beta estrogen receptor agonist, liquiritigenin, in addition to inhibiting beta amyloid peptide toxicity (Liu et al 2009), has been shown to promote neurogenesis by modulating the notch-2 signaling (Liu et al 2010). Liquiritigenin is a flavanoid extracted from Glycyrrhizae radix, a traditional Chinese medicine used to treat inflammation.

Sonic Hedgehog pathway (Shh). Sonic Hedgehog is a soluble extracellular signaling protein that is important for neurogenesis in the adult mammalian brain. In addition to increasing hippocampal progenitor cell proliferation in the DG, Shh promotes the self-renewal and proliferation of adult neural stem cells and regulates their cellular migration (Angot et al 2008; Ihrie et al 2011) Estrogen supplementation triggers the up-regulation of Shh (Koga et al 2008).

Neurotrophic factors. Of the four identified neurotrophic factors—brain derived neurotrophic factor (BDNF); nerve growth factor (NGF), neurotrophin 3 (NT-3) and neurotrophin 4/5 (NT-4/5)—it is mainly BDNF that has been linked to the activation of the various downstream effectors involved in neurogenesis. This occurs via the binding of BDNF to its tyrosine kinase receptor, TrkB. Studies have documented that functional TrkB signaling is required to stimulate the proliferation of neural stem cells in the hippocampus (Li et al 2008) and that the survival, dendritic arborization and functional integration of newborn neurons in the adult DG is dependent on TrkB receptor activity (Bergami et al 2008). The role of BDNF in the SVZ is less clear.

NGF does not have an effect on the proliferation of progenitor cells in the DG, but has been associated with the enhanced survival of neurons in the adult hippocampus (Frielingsdorf et al 2007). NT-3 has been shown to mediate spatial learning and memory in the adult brain (Shimazu et al 2006; Frielingsdorf et al 2007).

Vitamin D and Neurogenesis.

The human brain has established pathways for both the synthesis and degradation of vitamin D3 (Garcion et al; Eyles et al 2005). Clinical studies have confirmed a linkage with low vitamin D and cognitive impairment (Morris 1993) and with vitamin D treatment, slowing down the cognitive impairment and deterioration of patients with AD (Buell and Dawson-Hughes 2008). These clinical observations are supported by the demonstration of reduced mRNA levels of the vitamin D receptor (VDR) in the hippocampal CA1 and CA2 regions in post mortem AD brains (Sutherland et al 1992) and the increased frequency of VDR polymorphisms in AD brains compared with age-matched normal controls (Gezen-Ak 2007).

Stem cells and neural progenitor cells in the hippocampal dentate gyrus (DG) retain the ability to proliferate and develop into neurons in adults (Christie and Cameron 2006). Vitamin D3 deficiency promotes the death of newly generated neurons and their neurite growth (Brown et al 2003) before they reach maturation (Zhu et al 2012). This occurs as a result of a decline in the level of hippocampal NGF which is needed for the late stage of normal neurogenesis. The decrease in NGF is associated with a reduction of the neuronal 1 alpha (OH)ase (CYP27B1) gene (Zhu et al 2012) and, experimentally, is corrected by treatment with NGF. The latter is up-regulated by genistein as is the CYP27B1 gene responsible for the synthesis of vitamin D3 and its eventual cellular activity. Vitamin D3 also regulates other important cell functions such the multiple Ca++-dependent signaling processes.

In the methods described herein, estrogen supplementation stimulates the mRNA of both BDNF and NGF as does soy phytoestrogen. Vitamin D also regulates the synthesis of NGF and other neurotrophins: NT3 and Glial derived neurotrophic factor. Two of the proposed additives also function as neuropeptides: GLP-1 and NGF induced neurogenesis and caffeine increases hippocampal BDNF.

### Oxygen and the Regulation of Neurogenesis in Health and Disease.

The compositions described herein can be used to up regulate CNS acetylcholine synthesis. The compositions described herein can also be used to regulate blood flow and angiogenesis. The two major substrates for brain energy and cellular function are glucose and oxygen.

The brain consumes about 20% of the total body requirements at a relatively low oxygen tension: 27+−6 mmHg in the cerebral cortex and 20+−3 mmHg in the hippocampus (Ivanovic 2009). This so-called “physiologic hypoxia” is central to neurogensis and to the local brain demands of brain metabolic activity.

Within the stem cell “neurogenic niche” (in the DG and SVZ) is the “vascular niche”, comprised of blood vessels adjacent to and within the neuroblast complexes, and which serves as an essential component of the “oxygen niche” (Shen et al 2008). Dividing stem cells are closely apposed to the vascular endothelial cells.

Proliferation of neural stem cell (NSC) is promoted and apoptosis is reduced when in an environment of low O2 tension. This includes the differentiation of precursor cells into neurons with specific neurotransmitter function. Reduced oxygen levels also promote cell survival and proliferation of CNS stem cells (Morrison et al 2000). Hypoxia promotes the proliferation of NSC via the hypoxia-inducible transcription factor 1 alpha (HIF-1) (Zhao et al 2008; Panchision 2009). The following molecular mechanisms modify the behavior and function of NSC's in lower oxygen levels: Notch pathway (Diez et al 2007); Bone morphogenetic protein pathway (Pistollato et al 2007) and the Wnt/beta-catenin pathway (Jolly et al 2009).

Angiogenesis and endothelial function: Estrogen modulates and enhances pro-angiogenic molecular expression and thereby cerebral angionenesis, via the up regulation of factors such as vascular endothelial growth factor (Jesmin et al 2003).

Estrogen has an essential role in the regulation of endothelium dependent vasodilation and relaxation mediated by different endothelial-dependent relaxing factors including prostacyclin and nitric oxide (NO). Nitric oxide is formed from L-arginine by nitric oxide synthase (eNOS). Treatment with estrogen activates eNOS in cerebral blood vessels and an increase in cerebral blood flow (Stirone et al 2005).

The expression of eNOS in vascular smooth muscle is enhanced by ER beta and repressed by ER alpha (Tsutsumi et al 2008: see Cui et al). ER beta is the predominant estrogen receptor in the brain with a high affinity to the binding of estradiol and genistein (Cui et al 2013).

In addition, cholinergic pathways regulate cerebral vascular resistance, relaxation and regional blood flow (Sato et al 2004). This is also mediated via muscarinic Ach receptors that trigger the release of the actual relaxing factor: NO. Acetylcholine induced relaxation occurs in cerebral but not peripheral blood vessels (Yamada et al 2001).

In the methods claimed herein, the 17-beta estradiol and genistein up regulate CNS acetylcholine synthesis and regulates blood flow and angiogenesis.

### Sex Hormones and Brain Aging: Biosynthesis and Signaling Pathways in Health and Disease.

The compositions described herein can be used to regulate neural development, synaptic plasticity, and cell survival. The compositions can also be used to induce neuroprotective effects, improve learning, and inhibit memory loss.

The sex steroid, estrogen, in addition to its functions in female reproduction, have extragonal sites of steriodgenesis and activity via signaling pathways, principally through the classical nuclear receptors and cell surface membrane receptors.

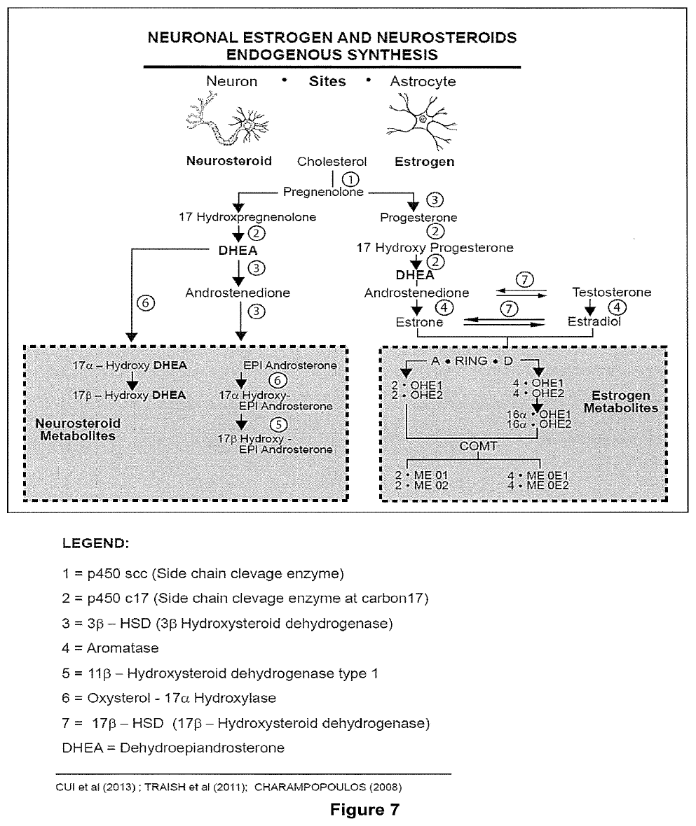

Estrogen Synthesis: The pathway for estrogen synthesis is summarized in FIG. 7. In premenopausal women the predominant estrogen is 17-beta estradiol (E2) whereas in postmenopausal women it is the less active estrogen, estrone (E1). Estrogen production starts with cholesterol and via the cytochrome P450 side chain cleavage enzyme (P450scc) catalyzed to progesterone, followed by conversion to progesterone (via 3-beta-hydroxysteroid dehydrogenase) and then to the androgen (androstenedione). The latter may be hydroxylated (via 17 beta hydoxysteroid dehydrogenase; 17-beta HSD) to testosterone or undergo aromatization to estrone. Estradiol is subsequently formed from either the 17-beta HSD of estrone and/or by aromatization of testosterone. The bioactivity of estrogen is subject to its peripheral metabolism and binding to organ specific tissue receptors.

The major site of estrogen synthesis in premenopausal women is primarily from the ovaries with small but significant amounts produced in non-gonad tissues including the brain. In post menopausal women, the major site of estrogen synthesis is in adipose tissue via the aromatization of adrenal dehydroepiandrosterone. This process increases with age (Misso et al). Aromatase activity has important role in the brain where it is potently inhibited by increased concentrations protein kinase dependent ATP, Mg++, or Ca++. Genistein (a tyrosine kinase inhibitor) blocks the ATP, Mg++ or Ca++ induced inhibition completely (Charlier et al 2011).

The brain has all of the enzymes required for the synthesis of estrogen from cholesterol as found in the hippocampus, amygdala, cerebral cortex and other relevant areas in the brain (Do Rego et al 2009). Cell specific E2 can be produced from both circulating E2 and from C19 steroid precursors that serve as substrates for brain estrogen synthesis (Kanecheva et al 2011). This takes place in neurons and astrocytes but not microglia or oligodendrocytes.

Brain estrogen is involved in the regulation of neural development, synaptic plasticity and cell survival (Azcoitia et al 2011).

Peripheral and Brain Estrogen Receptors: type, signaling and aging: The principal mode of estrogen signaling is via nuclear ER's (ER alpha; ER beta) and via subsequent activation of tissue specific target tissue factors, modulation and transcription of the respective organs specific gene function and/or via non-genomic membrane receptors such as GPR30. The mode of activation—seconds after cell membrane ER signaling (Maggiolini et al 2010) vs hours after the nuclear ER response—is factored into the disclosed formulation, designed to sequence the absorption of the three component ingredients in order to optimize the biologic efficacy of the respective ingredients [0034;0068]. The rapid non-genomic estrogen signaling is independent of ERalpha and ER beta (Thomas et al 2005).

ER alpha and ER beta co-localize in many cell types—including neurons and glial cells—and though they are encoded by separate genes (ESR1 and ESR2 respectively) there is functional cross talk between the two receptors (Enmark et al 1997). ER alpha is expressed primarily in the gonadal organs (and in those areas of the brain associated with neuroendocrine activity) and ER beta in non-gonadal tissues including the brain. ER beta concentrations are highest in the hippocampus, and cerebral cortex and are thus associated with mood and cognitive actions (Osterlund et al 2000).

Both ERs can stimulate or repress target gene transcription. Thus, the expression of inducible nitric oxide synthase in vascular smooth muscle (a positive effect) is increased by ER beta and decreased by ER alpha (Tsutsumi et al 2008).

Both ER's have a 97% homology with similar selectivity and affinity, when binding to their respective estrogen receptor elements (EREs) and co-promotor genes (Cui et al 2013). A significant difference is their effect on activator protein 1 (AP-1). ER alpha-AP-1 promotes breast cancer cell proliferation (via cyclin D1), whereas ER beta inhibits AP-1 dependent transcription of cyclin D1. Thus estrogens with a higher affinity for the ER beta receptor meet the criteria for classification as a SERM (selective estrogen receptor modulator) with predominant estrogen agonist activity in certain organs (brain and bone) and estrogen protective activity in the breast and endometrium. Isoflavones, for example genistein derived from soy (Clarkson et al 1995) and liquiritigenin (Mersereau et al 2008) have been shown to exhibit what some have termed NeuroSERM like activity (Zhao et al 2005).

The age related expression of ER alpha and ER beta differs: the ER alpha level in aging rats does not change whereas the ER beta level decreases significantly with advancing age (Sharma and Thakur 2006). This is reflected in an attenuated expression in hippocamapal spine synapse complexes, the associated markers of cholinergic activity and a decline both in cognitive function (Frick 2009; Foy 2011) and neural response to ET (Barron and Pike 2013).

This experimental data is consistent with the clinical “window of therapeutic opportunity” concept and the need for early vs late post menopausal ET (Sherwin and Henry 2008).

Estrogen loss and Alzheimer's Disease Risk.

The greater prevalence of AD in women (Plassman et al 2007) may be attributable to both the dramatic decrease in estrogen production following menopause and their longer life span, resulting in an extended period lived in an environment of sex steroid depletion. This may also account for a greater severity of cognitive deficits and beta amyloid neuropathology in women compared to men with AD (Barnes et al 2005).

Estrogen Reduces Beta-Amyloid Levels: Whereas a recent study demonstrated no relationship between brain estrogen levels and neuropathology in normal elderly women, the concentration of estrogen is reduced in the brains of older women with AD (Rosario et al 2011).

Estrogen is implicated in the regulation of beta amyloid production, including the processing of the amyloid precursor protein (APP) and in its clearance from the CNS. APP is metabolized by two competing pathways: the beta secretase (BACE) route, which cleaves APP into the toxic accumulation of beta amyloid comprising two species that are 40 and 42 amino acids in length, and the non-amyloidogenic alpha secretase pathway. This route prevents formation of the full-length amyloid peptide resulting in a more soluble protective form of APP, App alpha (Jaffe et al 1994).

This is said to occur via an ER independent mechanism involving the mitogen activated protein kinase (MAPK) and other extracellular-regulated kinases (Manthey et al 2001; Zhang et al 2005). Estrogen may also inhibit the expression of BACE (Amtul et al 2010) and the availability of the APP precursor substrate (Geenfield et al 2002).

Estrogen modulates beta amyloid clearance by stimulating microglial phagocytosis (Li et al 2000) and the degradation of beta amyloid peptide monomers and oligomers by a variety of beta amyloid degrading enzymes, including both an insulin degrading enzyme and angiotensin converting enzyme (Leissring et al 2008). This may have clinical implications given the increased risk of AD in women with insulin resistance and hypertension.

In addition to the above, studies involving two phytoestrogens—genistein (Oh et al 2004) and liquiritigenin (Liu et al 2009) have both confirmed amyloid-induced neuroprotective effects with an improvement in learning and memory deficits (Liu et al 2010) and an associated SERM like protection of the endometrium (Oh et al 2004). These actions are complementary to that of Huperzine A, vitamin D, and caffeine.

### EXAMPLES

The examples illustrate exemplary methods provided herein. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### Example 1: Translational Pharmacokinetic and Pharmacodynamic Studies

Goal: To apply experimental proof of concept principles by combining selected botanical and natural compounds and/or their synthetic derivatives, and to demonstrate their systemic bioavailability (pharmacokinetics) and bioactivity as brain health modulators (pharmcodynamics) in adult women with and without symptoms of cognitive, memory and/or mood impairment, and as promoters of healthy brain aging.

Rationale: The study design allows assessment of the combined effects of natural preparations and/or synthetic derivatives of soy isoflavones (genistein), Huperzine A and vitamin D (Broad Based Balanced Bioactive Brain Blend™ BBBBB™) with a caffeine and/or other additives on varying aspects of cognition, executive function and memory. The advantage of the BBBBB™ is based on the combination's additive and/or synergistic bioactivity of each ingredient's independent effect on relevant aspects of the multiple molecular signaling pathways involved in brain health and function, including but not limited to the non-amyloidogenic metabolism of amyloid precursor protein (APP).

Assessments are based on the evaluation of differing dosage regimens to meet the clinical needs of women with asymptomatic physiologic brain aging, those with accelerated and symptomatic change (benign senescent forgetfulness), and women at risk of developing mild cognitive impairment (MCI).

The studies include the measurement of each ingredient's pharmacokinetic profile based on the BBBBB™'s proprietary sequenced and time released formulation, and the standardized assay of biomarkers associated with neurotrophic function, neurotransmission and brain health protective activity. Clinical response is based on standardized neuropsychologic tests sensitive to the effects of the BBBBB™ ingredients.

Pharmacokinetic Study: To measure the absorption, bioavailability and bioactivity of two strengths of the BBBBB™ with a caffeine additive in healthy post menopausal female volunteers.

Aim: To determine the blood levels of each of the BBBBB™ proprietary formulated ingredients with specific assays (Huperzine A; genistein; 25 (OH) vitamin D3) plus caffeine over a 48 hour time interval, and to assay alterations in the biomarkers of two neurotrophic proteins: brain derived neurotrophic factor (BDNF) and Nerve Growth Factor (NGF); two biomarkers of acetylcholine metabolism: Choline acetyltransferase (ChAT) and Acetylcholinesterase (AChE); biomarker of Wnt/beta catenin: Dkk1.

Study Subjects:

Ten healthy adult female subjects aged 40 to 60 years.

Postmenopausal for at least 12 months or post total hysterectomy with bilateral oophorectomy, confirmed with a plasma FSH level >50 miu.

Currently not using a vitamin D supplement with at least a one month washout period.

Study design: Randomized four way open label crossover under fasting conditions of four prototype BBBBB™ regimens designated A, B, C and D. 1.

### Proprietary BBBBB™ Formulations.

Immediate but sequenced released (ISR) twice daily product dosing:

A: Soy Isoflavone 55 mg, Vitamin D3 600iu, Huperzine A 100 mcg, caffeine 75 mg (AM dose).

Soy Isoflavone 55 mg, Vitamin D3 600 iu, Huperzine A 75 mcg (PM dose)

B: Soy Isoflavone 55 mg, Vitamin D3 600iu, Huperzine A 100 mcg, caffeine 75 mg (AM dose).

Soy Isoflavone 55 mg, vitamin D3 600 iu, Huperzine A 175 mcg (PM dose).

Extended sequenced release (ESR) once daily product dosing:

C. Soy Isoflavones 110 mg, Vitamin D3 1200 mg, Huperzine A 175 mcg, caffeine 75 mg.

D. Soy Isoflavones 110 mg, Vitamin D3 1200 mg, Huperzine A 275 mcg, caffeine 75 mg.

Dosing regimen: For the ISR product a single capsule of the AM dose test product is taken with 8 ounces of room temperature water after an overnight fast of at least 10 hours at 8 am with the PM dose at 8 PM also with an 8 fluid ounce of room temperature water.

Washout: At least 7 days between each test period.

Confinement: At least 10 hours prior to dosing and 48 hours after each dosing period.

pK sampling: 12 blood samples per subject for each dosing period for later biochemical analysis. Times: 120 minutes prior to dosing then at 0.25; 0.05; 0.75; 1.0; 2.0; 4.0; 6.0; 8.0; 10.0; 12.0; 24.0; 48.0 hours post dose.

Pharmacokinetic and Statistical Data Analysis.

Pharmacokinetics: analysis is performed using standard non-compartmental methods.

Statistics: Analysis is performed using SAS and 90% confidence interval and ratios for relative mean in transformed AUC 0-t; AUC 0-∞, and Cmax of each test formulation is calculated.

Bioanalysis of samples: Plasma levels of Huperzine A is assayed by a Huperzine A specific bioassay developed at the University of Florida's Department of Pharmaceutics using HPLC/MS/MS technology (Gunther Hauchaus, Director); the other biomarkers are assayed utilizing standardized sandwich ELISA methodology at the University of California's Clinical and Translational Research Institute (Michael Rosenbach, Director)

Pharmacodynamic Study: Aim is to evaluate 90 early postmenopausal women with subjective memory/cognitive complaints in a randomized blinded three way placebo-controlled study comparing the placebo group (30 women) with two equally matched groups: 30 women randomized to receiving test product B (see before) and another 30 women randomized to product D (See before).

Study group criteria: as before

Treatment Duration: 12 weeks.

Procedure:

Cognitive assessments take place at baseline, after 8 weeks and 12 weeks of treatment with either the placebo or product B or product D. Tests include but are not limited to: a measure of immediate verbal memory (paragraph recall from the Wechsler Memory Scale III); a measure of executive function (Intradimensional/extradimensional Shift Measure from the CANTAB battery); Clinical Dementia Rating Scale (CDR); Mini-mental State Examination (MMSE).

Biomarkers are similarly measured at baseline, and after 8 and 12 weeks of treatment with either placebo or the two treatment products. These include:

Plasma ingredient levels: Huperzine A; Genistein; 25 (OH) vitamin D3; Caffeine.

Neurotrophins and neurotransmitters: BDNF; NGF; ChAT; AchE; Dkk1.

APP metabolism: Assays measuring the expression of alpha secretase and beta gamma secretase enzyme activity and SIRT1.

Inflammatory cytokines panel: IL-1; IL-2; IL-4; IL-8; IL-10; IL-13; TNF-alpha; osteopontin; and two anti-inflamatory markers G-CSF and Fetuin-A. These assays are performed utilizing human ELISA (enzyme linked immunosorbent assays) kits.

Oxidative stress: urinary 8-epi-prostaglandin F2 alpha using Immunoaffinity Extraction—Gas-Chromatography—Negative Ion Chemical Ionization Mass Spectrometry.

Glucose Tolerance Tests: a 10 subject subset per group is selected for a 100 gram two hour glucose tolerance tests at baseline and 12 weeks. This includes blood glucose and insulin assays utilizing standard assay technology.

Bioanalysis: The huperzine assay is performed at the University of Florida Department of Pharmaceutics (see before); the oxidative stress test at the University of Florida Department of Pharmacotherapy and Translational research (John S. Markowitz, Director) and all ELISA based tests at the University of California San Diego (see before).

Power Analysis is based on the study by File et al (2005) which determined that a sample size of 25 women in each group would allow for 74% power to test the main effect of each treatment group compared with the placebo group (File et al Menopause 2005; 12: 193-201).

### Example 2: Effect of Composition on Working Memory of Chronic Estrogen-Deficient Rats

A. Objectives: This study investigates whether the disclosed composition can reverse working memory deficiency in aged ovariectomized rats and, further, whether the treatments of rats with the disclosed composition would result in improved working memory of rats.

B. Materials and Methods

Animals: Thirty-six retired breeder female rats (8-10 months old) are purchased from Harlan Sprague Dawley, Inc. The rats are housed in separate cages and are initially maintained on a 12:12 hour light/dark cycle with access to Chow diet and water ad libitum. After bilateral ovariectomy, the rats are fed with a casein/lactalbumin-based control diet for about 12 months (until about 2 years of age). The rats are then evaluated in a 8-arm radial arm maze to determine their baseline working memory. The rats are then randomized into one of 3 groups and are fed with the control diet (Ctl), or the control diet supplemented with the disclosed composition. The rats are tested in the maze at 1 and 3 months after the initiation of the treatments.

After 3 months of treatment, each of the 3 groups are divided randomly into two subgroups. One subgroup was given the disclosed composition in addition to their control regimen and the other subgroup received only the their control regimen. After 3 weeks of supplementation with control regimen, working memory is reevaluated.

### Radial Arm Maze Training

A pellet of fudge brownie (Little Debbie, Mckee Foods, Collegedale, Tenn.) is placed in each of the 8 food wells located at the end of each arm to serve as a reward. The rats are allowed to explore the maze for 10 min or until all eight rewards were eaten. The training session is terminated when the rats are able to eat all 8 rewards within 10 min.

### Radial Arm Maze Test

After the training, the rats are tested once per day, for four consecutive days per week for two weeks. One day prior to the test, and during the first three test days, the food intake is reduced to 25% of the normal 40 g/day food intake. A visit to an arm is recorded if the rat reached three-fourths of the length of the arm. The maze performance is recorded as the number of correct choices in the first 8 visits. A mistake is counted if a rat reentered an arm from which the rat has already eaten the bait. A test session of a rat is terminated when the rat ate all eight rewards or 10 min has elapsed. If a rat has a perfect working memory, the rat should score 8 correct choices in the first 8 visits (or eat all eight baits without re-visiting an arm from which the rat has already eaten the bait). The number of correct choices in the first 8 visits equals 8 minus the number of mistakes in the first 8 visits. After the test, the mean of the 8 test results of a given rat is used in the statistical analyses.

Uterus and Body Weight: The body weights of the rats are recorded every two weeks during the study and at necropsy. At the end of the study, the rats are euthanized with pentobarbital (100 mg/kg). The uteri are collected and their weights are determined with an electronic balance.

Statistical Analyses: All data are analyzed using BMDP Statistical Software, version 7.0 (Los Angeles, Calif.).

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

### REFERENCES

1. Abreu R V, Silva-Oliveira E M, Moraes M F, et al. Chronic coffee and caffeine ingestion effects on the cognitive function and antioxidant system of rat brains. Pharmacology, Biochemistry, and Behavior. 2011; 99(4):659-664.
2. Adachi K, Mirzadeh Z, Sakaguchi M, et al. Beta-catenin signaling promotes proliferation of progenitor cells in the adult mouse subventricular zone. Stem Cells (Dayton, Ohio). 2007; 25(11):2827-2836.
3. Adan A, Serra-Grabulosa J M. Effects of caffeine and glucose, alone and combined, on cognitive performance. Human Psychopharmacology. 2010; 25(4):310-317.
4. Aloe L, Rocco M L, Bianchi P, et al. Nerve growth factor: from the early discoveries to the potential clinical use. Journal of Translational Medicine. 2012; 10:239.
5. Alvarez A, Alarcon R, Opazo C, et al. Stable complexes involving acetylcholinesterase and amyloid-beta peptide change the biochemical properties of the enzyme and increase the neurotoxicity of Alzheimer's fibrils. The Journal of Neuroscience: the official journal of the Society for Neuroscience. 1998; 18(9):3213-3223.
6. Alvarez A R, Godoy J A, Mullendorff K, et al. Wnt-3a overcomes beta-amyloid toxicity in rat hippocampal neurons. Experimental Cell Research. 2004; 297(1):186-196.
7. Alvarez G, Munoz-Montano J R, Satrustegui J, et al. Lithium protects cultured neurons against beta-amyloid-induced neurodegeneration. FEBS letters. 1999; 453(3):260-264.
8. Alzoubi K H, Srivareerat M, Aleisa A M, et al. Chronic caffeine treatment prevents stress-induced LTP impairment: the critical role of phosphorylated CaMKII and BDNF. Journal of Molecular Neuroscience: MN. 2013; 49(1):11-20.
9. Amtul Z, Wang L, Westaway D, et al. Neuroprotective mechanism conferred by 17beta-estradiol on the biochemical basis of Alzheimer's disease. Neuroscience. 2010; 169(2):781-786.
10. Angot E, Loulier K, Nguyen-Ba-Charvet K T, et al. Chemoattractive activity of sonic hedgehog in the adult subventricular zone modulates the number of neural precursors reaching the olfactory bulb. Stem Cells (Dayton, Ohio). 2008; 26(9):2311-2320.
11. Annweiler C, Beauchet O. Vitamin D-mentia: randomized clinical trials should be the next step. Neuroepidemiology. 2011; 37(3-4):249-258.
12. Annweiler C, Schott A M, Allah G, et al. Association of vitamin D deficiency with cognitive impairment in older women: cross-sectional study. Neurology. 2010; 74(1):27-32.
13. Anupongsanugool E, Teekachunhatean S, Rojanasthien N, et al. Pharmacokinetics of isoflavones, daidzein and genistein, after ingestion of soy beverage compared with soy extract capsules in postmenopausal Thai women. BMC Clinical Pharmacology. 2005; 5(1):2.
14. Arab L, Biggs M L, O'Meara E S, et al. Gender differences in tea, coffee, and cognitive decline in the elderly: the Cardiovascular Health Study. Journal of Alzheimer's disease: JAD. 2011; 27(3):553-566.
15. Arendash G W, Cao C. Caffeine and coffee as therapeutics against Alzheimer's disease. Journal of Alzheimer's disease: JAD. 2010; 20 Suppl 1:S117-126.
16. Arendash G W, Mori T, Cao C, et al. Caffeine reverses cognitive impairment and decreases brain amyloid-beta levels in aged Alzheimer's disease mice. Journal of Alzheimer's Disease: JAD. 2009; 17(3):661-680.
17. Atlante A, Bobba A, Paventi G, et al. Genistein and daidzein prevent low potassium-dependent apoptosis of cerebellar granule cells. Biochemical Pharmacology. 2010; 79(5):758-767.
18. Azcoitia I, Yague J G, Garcia-Segura L M. Estradiol synthesis within the human brain. Neuroscience. 2011; 191:139-147.
19. Balion C, Griffith L E, Strifler L, et al. Vitamin D, cognition, and dementia: a systematic review and meta-analysis. Neurology. 2012; 79(13):1397-1405.
20. Bang O Y, Hong H S, Kim D H, et al. Neuroprotective effect of genistein against beta amyloid-induced neurotoxicity. Neurobiology of Disease. 2004; 16(1):21-28.
21. Banks W A, Farr S A, Morley J E. Entry of blood-borne cytokines into the central nervous system: effects on cognitive processes.

Neuroimmunomodulation. 2002; 10(6):319-327.
22. Bansode S S, Banarjee S K, Gaikwad D D, et al. Microencapsulation: A Review. International Journal of Pharmaceutical Sciences Review and Research. 2010; 1(2):38.
23. Barnes L L, Wilson R S, Bienias J L, et al. Sex differences in the clinical manifestations of Alzheimer disease pathology. Archives of General Psychiatry. 2005; 62(6):685-691.
24. Bateman R J, Blazey T M, Holtzman D M, et al. Clinical and biomarker changes in dominantly inherited Alzheimer's disease. The New England Journal of Medicine. 2012; 367(9):795.
25. Behloul N, Wu G. Genistein: a promising therapeutic agent for obesity and diabetes treatment. European Journal of Pharmacology. 2013; 698(1-3):31-38.
26. Bergami M, Rimondini R, Santi S, et al. Deletion of TrkB in adult progenitors alters newborn neuron integration into hippocampal circuits and increases anxiety-like behavior. Proceedings of the National Academy of Sciences of the United States of America. 2008; 105(40):15570-15575.
27. Bhukhai K, Suksen K, Bhummaphan N, et al. A phytoestrogen diarylheptanoid mediates estrogen receptor/Akt/glycogen synthase kinase 3beta proteindependent activation of the Wnt/beta-catenin signaling pathway. The Journal of Biological Chemistry. 2012; 287(43):36168-36178.
28. Bitto A, Granese R, Triolo 0, et al. Genistein aglycone: a new therapeutic approach to reduce endometrial hyperplasia. Phytomedicine: International Journal of Phytotherapy and Phytopharmacology. 2010; 17(11):844-850.
29. Bloedon L T, Jeffcoat A R, Lopaczynski W, et al. Safety and pharmacokinetics of purified soy isoflavones: single-dose administration to postmenopausal women. The American Journal of Clinical Mutrition. 2002; 76(5):1126-1137.
30. Bragdon B, Moseychuk O, Saldanha S, et al. Bone morphogenetic proteins: a critical review. Cellular Signalling. 2011; 23(4):609-620.
31. Breunig J J, Silbereis J, Vaccarino F M, et al. Notch regulates cell fate and dendrite morphology of newborn neurons in the postnatal dentate gyrus. Proceedings of the National Academy of Sciences of the United States of America. 2007; 104(51):20558-20563.
32. Brewer L D, Thibault V, Chen K C, et al. Vitamin D hormone confers neuroprotection in parallel with downregulation of L-type calcium channel expression in hippocampal neurons. The Journal of Neuroscience: the official Journal of the Society for Neuroscience. 2001; 21(1):98-108.
33. Brinton R D. Estrogen-induced plasticity from cells to circuits: predictions for cognitive function. Trends in Pharmacological Sciences. 2009; 30(4):212-222.
34. Brown J, Bianco J I, McGrath J J, et al. 1,25-dihydroxyvitamin D3 induces nerve growth factor, promotes neurite outgrowth and inhibits mitosis in embryonic rat hippocampal neurons. Neuroscience Letters. 2003; 343(2):139-143.
35. Brown R J, Walter M, Rother K I. Ingestion of diet soda before a glucose load augments glucagon-like peptide-1 secretion. Diabetes Care. 2009; 32(12):2184-2186.
36. Buell J S, Dawson-Hughes B. Vitamin D and neurocognitive dysfunction: preventing “D”ecline? Molecular Aspects of Medicine. 2008; 29(6):415-422.
37. Buteau J, El-Assaad W, Rhodes C J, et al. Glucagon-like peptide-1 prevents beta cell glucolipotoxicity. Diabetologia. 2004; 47(5):806-815.
38. Cappuccio I, Calderone A, Busceti C L, et al. Induction of Dickkopf-1, a negative modulator of the Wnt pathway, is required for the development of ischemic neuronal death. The Journal of Neuroscience: the official journal of the Society for Neuroscience. 2005; 25(10):2647-2657.
39. Caricasole A, Copani A, Caraci F, et al. Induction of Dickkopf-1, a negative modulator of the Wnt pathway, is associated with neuronal degeneration in Alzheimer's brain. The Journal of Neuroscience: the official journal of the Society for Neuroscience. 2004; 24(26):6021-6027.
40. Cerpa W, Gambrill A, Inestrosa N C, et al. Regulation of NMDA-receptor synaptic transmission by Wnt signaling. The Journal of Neuroscience: the official journal of the Society for Neuroscience. 2011; 31(26):9466-9471.
41. Charlier T D, Cornil C A, Ball G F, et al. Diversity of mechanisms involved in aromatase regulation and estrogen action in the brain. Biochimica et Biophysica Acta. 2010; 1800(10):1094-1105.
42. Charlier T D, Harada N, Balthazart J, et al. Human and quail aromatase activity is rapidly and reversibly inhibited by phosphorylating conditions. Endocrinology. 2011; 152(11):4199-4210.
43. Chen J, Park C S, Tang S J. Activity-dependent synaptic Wnt release regulates hippocampal long term potentiation. The Journal of Biological Chemistry. 2006; 281(17):11910-11916.
44. Chen W Y, Bertone-Johnson E R, Hunter D J, et al. Associations between polymorphisms in the vitamin D receptor and breast cancer risk. Cancer Epidemiology, Biomarkers & Prevention: a publication of the American Association for Cancer Research, cosponsored by the American Society of Preventive Oncology. 2005; 14(10):2335-2339.
45. Chen X, Ghribi O, Geiger J D. Caffeine protects against disruptions of the bloodbrain barrier in animal models of Alzheimer's and Parkinson's diseases. Journal of Alzheimer's disease: JAD. 2010; 20 Suppl 1:S127-141.
46. Cheng C M, Cohen M, Wang J, et al. Estrogen augments glucose transporter and IGF1 expression in primate cerebral cortex. FASEB journal: official publication of the Federation of American Societies for Experimental Biology. 2001; 15(6):907-915.
47. Choe Y, Kozlova A, Graf D, et al. Bone morphogenic protein signaling is a major determinant of dentate development. The Journal of Neuroscience: the official journal of the Society for Neuroscience. 2013; 33(16):6766-6775.
48. Chowen J A, de Fonseca F R, Alvarez E, et al. Increased glucagon-like peptide-1 receptor expression in glia after mechanical lesion of the rat brain. Neuropeptides. 1999; 33(3):212-215.
49. Christie B R, Cameron H A. Neurogenesis in the adult hippocampus. Hippocampus. 2006; 16(3):199-207.
50. Clarkson T B, Anthony M S, Hughes C L, Jr. Estrogenic soybean isoflavones and chronic disease Risks and benefits. Trends in Endocrinology and Metabolism: TEM. 1995; 6(1):11-16.
51. Clarkson T B, Utian W H, Barnes S, et al. The role of soy isoflavones in menopausal health: report of The North American Menopause Society/Wulf H. Utian Translational Science Symposium in Chicago, Ill. (October 2010). Menopause (New York, N.Y.). 2011; 18(7):732-753.
52. Colak D, Mori T, Brill M S, et al. Adult neurogenesis requires Smad4-mediated bone morphogenic protein signaling in stem cells. The Journal of Neuroscience the official journal of the Society for Neuroscience. 2008; 28(2):434-446.
53. Corley J, Jia X, Kyle J A, et al. Caffeine consumption and cognitive function at age 70: the Lothian Birth Cohort 1936 study. Psychosomatic Medicine. 2010; 72(2):206-214.
54. Cornet A, Baudet C, Neveu I, et al. 1,25-Dihydroxyvitamin D3 regulates the expression of VDR and NGF gene in Schwann cells in vitro. Journal of Neuroscience Research. 1998; 53(6):742-746.
55. Cross H S, Kallay E, Lechner D, et al. Phytoestrogens and vitamin D metabolism: a new concept for the prevention and therapy of colorectal, prostate, and mammary carcinomas. The Journal of Nutrition. 2004; 134(5):1207S-1212S.
56. Cui J, Shen Y, Li R. Estrogen synthesis and signaling pathways during aging: from periphery to brain. Trends in Molecular Medicine. 2013; 19(3):197-209.
57. Dai J, Li Y, Zhou H, et al. Genistein promotion of osteogenic differentiation through BMP2/SMAD5/RUNX2 signaling. International Journal of Biological Sciences. 2013; 9(10):1089-1098.
58. Dammann K W, Bell M, Kanter M, et al. Effects of consumption of sucromalt, a slowly digestible carbohydrate, on mental and physical energy questionnaire responses. Nutritional Neuroscience. 2012.
59. Dawson V L, Dawson™. Nitric oxide actions in neurochemistry. Neurochemistry International. 1996; 29(2):97-110.
60. De Ferrari G V, Chacon M A, Barria M I, et al. Activation of Wnt signaling rescues neurodegeneration and behavioral impairments induced by beta-amyloid fibrils. Molecular Psychiatry. 2003; 8(2):195-208.
61. de la Monte S M. Brain insulin resistance and deficiency as therapeutic targets in Alzheimer's disease. Current Alzheimer Research. 2012; 9(1):35-66.
62. Deltheil T, Guiard B P, Cerdan J, et al. Behavioral and serotonergic consequences of decreasing or increasing hippocampus brain-derived neurotrophic factor protein levels in mice. Neuropharmacology. 2008; 55(6):1006-1014.
63. Diez H, Fischer A, Winkler A, et al. Hypoxia-mediated activation of D114-Notch-Hey2 signaling in endothelial progenitor cells and adoption of arterial cell fate. Experimental Cell Research. 2007; 313(1):1-9.
64. Ding Q, Vaynman S, Akhavan M, et al. Insulin-like growth factor I interfaces with brain-derived neurotrophic factor-mediated synaptic plasticity to modulate aspects of exercise-induced cognitive function. Neuroscience. 2006; 140(3):823-833.
65. Diogenes M J, Costenla A R, Lopes L V, et al. Enhancement of LTP in aged rats is dependent on endogenous BDNF. Neuropsychopharmacology: official publication of the American College of Neuropsychopharmacology. 2011; 36(9):1823-1836.
66. Do Rego J L, Seong J Y, Buret D, et al. Neurosteroid biosynthesis: enzymatic pathways and neuroendocrine regulation by neurotransmitters and neuropeptides. Frontiers in Neuroendocrinology. 2009; 30(3):259-301.
67. Dong X C. Sirtuin biology and relevance to diabetes treatment. Diabetes Management (London, England). 2012; 2(3):243-257.
68. Dormire S L. The potential role of glucose transport changes in hot flash physiology: a hypothesis. Biological Research for Nursing. 2009; 10(3):241-247.
69. Dringen R, Gutterer J M, Hirrlinger J. Glutathione metabolism in brain metabolic interaction between astrocytes and neurons in the defense against reactive oxygen species. European journal of biochemistry/FEBS. 2000; 267(16):4912-4916.
70. Duarte A I, Moreira P I, Oliveira C R. Insulin in central nervous system: more than just a peripheral hormone. Journal of Aging Research. 2012:384017.
71. Duffy R, Wiseman H, File S E. Improved cognitive function in postmenopausal women after 12 weeks of consumption of a soya extract containing isoflavones. Pharmacology, Biochemistry, and Behavior. 2003; 75(3):721-729.
72. Durk M R, Chan G N, Campos C R, et al. 1alpha,25-Dihydroxyvitamin D3-liganded vitamin D receptor increases expression and transport activity of P-glycoprotein in isolated rat brain capillaries and human and rat brain microvessel endothelial cells. Journal of Neurochemistry. 2012; 123(6):944-953.
73. Eagan D E, Gonzales M M, Tarumi T, et al. Elevated serum C-reactive protein relates to increased cerebral myoinositol levels in middle-aged adults. Cardiovascular Psychiatry and Neurology. 2012; 2012.
74. Eikelenboom P, Veerhuis R. The role of complement and activated microglia in the pathogenesis of Alzheimer's disease. Neurobiology of Aging. 1996; 17(5):673-680.
75. Encinas J M, Sierra A, Valcarcel-Martin R, et al. A developmental perspective on adult hippocampal neurogenesis. International Journal of Developmental Neuroscience: the official journal of the International Society for Developmental Neuroscience. 2013; 31(7):640-645.
76. Enmark E, Pelto-Huikko M, Grandien K, et al. Human estrogen receptor betagene structure, chromosomal localization, and expression pattern. The Journal of Clinical Endocrinology and Metabolism. 1997; 82(12):4258-4265.
77. Erickson K I, Colcombe S J, Raz N, et al. Selective sparing of brain tissue in postmenopausal women receiving hormone replacement therapy. Neurobiology of Aging. 2005; 26(8):1205-1213.
78. Eriksson P S, Perfilieva E, Bjork-Eriksson T, et al. Neurogenesis in the adult human hippocampus. Nature Medicine. 1998; 4(11):1313-1317.
79. Eskelinen M H, Kivipelto M. Caffeine as a protective factor in dementia and Alzheimer's disease. Journal of Alzheimer's disease: JAD. 2010; 20 Suppl 1:S167-174.
80. Eskelinen M H, Ngandu T, Tuomilehto J, et al. Midlife coffee and tea drinking and the risk of late-life dementia: a population-based CAIDE study. Journal of Alzheimer's Disease: JAD. 2009; 16(1):85-91.
81. Evans M, Elliott J G, Sharma P, et al. The effect of synthetic genistein on menopause symptom management in healthy postmenopausal women: a multicenter, randomized, placebo-controlled study. Maturitas. 2011; 68(2):189-196.
82. Eyles D W, Smith S, Kinobe R, et al. Distribution of the vitamin D receptor and 1 alpha-hydroxylase in human brain. Journal of Chemical Neuroanatomy. 2005; 29(1):21-30.
83. Faigle R, Song H. Signaling mechanisms regulating adult neural stem cells and neurogenesis. Biochimica et Biophysica acta. 2013; 1830(2):2435-2448.
84. Fiorelli G, Picariello L, Martineti V, et al. Estrogen synthesis in human colon cancer epithelial cells. The Journal of Steroid Biochemistry and Molecular Biology. 1999; 71(5-6):223-230.
85. Frick K M. Estrogens and age-related memory decline in rodents: what have we learned and where do we go from here? Hormones and Behavior. 2009; 55(1):2-23.
86. Friedman S M, Menzies I B, Bukata S V, et al. Dementia and hip fractures: development of a pathogenic framework for understanding and studying risk. Geriatric Orthopaedic Surgery & Rehabilitation. 2010; 1(2):52-62.
87. Frielingsdorf H, Simpson D R, Thal L J, et al. Nerve growth factor promotes survival of new neurons in the adult hippocampus. Neurobiology of Disease. 2007; 26(1):47-55.
88. Gao X, Liu K, Huang F, et al. Positive and negative regulation of insulin action by genistein in the endothelium. The Journal of Nutritional Biochemistry. 2013; 24(1):222-230.
89. Garcion E, Sindji L, Montero-Menei C, et al. Expression of inducible nitric oxide synthase during rat brain inflammation: regulation by 1,25-dihydroxyvitamin D3. Glia. 1998; 22(3):282-294.
90. Garcion E, Wion-Barbot N, Montero-Menei C N, et al. New clues about vitamin D functions in the nervous system. Trends in endocrinology and metabolism: TEM. 2002; 13(3):100-105.
91. Ge Y W, Lahiri D K. Regulation of promoter activity of the APP gene by cytokines and growth factors: implications in Alzheimer's disease. Annals of the New York Academy of Sciences. 2002; 973:463-467.
92. Geula C, Mesulam M M. Cholinesterases and the pathology of Alzheimer disease. Alzheimer Disease and Associated Disorders. 1995; 9 Suppl 2:23-28.
93. Gezen-Ak D, Dursun E, Ertan T, et al. Association between vitamin D receptor gene polymorphism and Alzheimer's disease. The Tohoku Journal of Experimental Medicine. 2007; 212(3):275-282.
94. Ghribi O. Potential mechanisms linking cholesterol to Alzheimer's disease-like pathology in rabbit brain, hippocampal organotypic slices, and skeletal muscle. Journal of Alzheimer's disease: JAD. 2008; 15(4):673.
95. Gibbs R B, Wu D, Hersh L B, et al. Effects of estrogen replacement on the relative levels of choline acetyltransferase, trkA, and nerve growth factor messenger RNAs in the basal forebrain and hippocampal formation of adult rats.

Experimental Neurology. 1994; 129(1):70-80.
96. Gilad L A, Bresler T, Gnainsky J, et al. Regulation of vitamin D receptor expression via estrogen-induced activation of the ERK 1/2 signaling pathway in colon and breast cancer cells. The Journal of Endocrinology. 2005; 185(3):577-592.
97. Gilad L A, Tirosh O, Schwartz B. Phytoestrogens regulate transcription and translation of vitamin D receptor in colon cancer cells. The Journal of Endocrinology. 2006; 191(2):387-398.
98. Gilbert E R, Liu D. Anti-diabetic functions of soy isoflavone genistein: mechanisms underlying its effects on pancreatic beta-cell function. Food & Function. 2013; 4(2):200-212.
99. Gillies G E, McArthur S. Estrogen actions in the brain and the basis for differential action in men and women: a case for sex-specific medicines. Pharmacological Reviews. 2010; 62(2):155-198.
100. Glorioso C, Sibille E. Between destiny and disease: genetics and molecular pathways of human central nervous system aging. Progress in Neurobiology. 2011; 93(2):165-181.
101. Gomez-Pinilla F, Vaynman S, Ying Z. Brain-derived neurotrophic factor functions as a metabotrophin to mediate the effects of exercise on cognition. The European Journal of Neuroscience. 2008; 28(11):2278-2287.
102. Greenfield J R, Farooqi I S, Keogh J M, et al. Oral glutamine increases circulating glucagon-like peptide 1, glucagon, and insulin concentrations in lean, obese, and type 2 diabetic subjects. The American Journal of Clinical Nutrition. 2009; 89(1):106-113.
103. Griffin W S. Inflammation and neurodegenerative diseases. The American Journal of Clinical Nutrition. 2006; 83(2):470S-474S.
104. Grysman A, Carlson T, Wolever T M. Effects of sucromalt on postprandial responses in human subjects. European Journal of Clinical Nutrition. 2008; 62(12):1364-1371.
105. Guarente L. Sirtuins, Aging, and Medicine. New England Journal of Medicine. 2011; 364(23):2235-2244.
106. Guarino M P, Ribeiro M J, Sacramento J F, et al. Chronic caffeine intake reverses age-induced insulin resistance in the rat: effect on skeletal muscle Glut4 transporters and AMPK activity. Age (Dordrecht, Netherlands). 2012:1755-1765.
107. Guillozet A L, Smiley J F, Mash D C, et al. Butyrylcholinesterase in the life cycle of amyloid plaques. Annals of Neurology. 1997; 42(6):909-918.
108. Gunn I, O'Shea D, Turton M D, et al. Central glucagon-like peptide-I in the control of feeding. Biochemical Society transactions. 1996; 24(2):581-584.
109. Han M E, Kim H J, Lee Y S, et al. Regulation of cerebrospinal fluid production by caffeine consumption. BMC Neuroscience. 2009; 10:110.
110. Harkavyi A, Whitton P S. Glucagon-like peptide 1 receptor stimulation as a means of neuroprotection. British Journal of Pharmacology. 2010; 159(3):495-501.
111. Harms L R, Burne T H, Eyles D W, et al. Vitamin D and the brain. Best practice & research. Clinical Endocrinology & Metabolism. 2011; 25(4):657-669.
112. Hartz A M, Bauer B. Regulation of ABC transporters at the blood-brain barrier: new targets for CNS therapy. Molecular Interventions. 2010; 10(5):293-304.
113. Harvey B K, Hoffer B J, Wang Y. Stroke and TGF-beta proteins: glial cell linederived neurotrophic factor and bone morphogenetic protein. Pharmacology & Therapeutics. 2005; 105(2):113-125.
114. He P, Shen Y. Interruption of beta-catenin signaling reduces neurogenesis in Alzheimer's disease. The Journal of Neuroscience: the official journal of the Society for Neuroscience. 2009; 29(20):6545-6557.
115. Heaney R P, Armas L A, Shary J R, et al. 25-Hydroxylation of vitamin D3: relation to circulating vitamin D3 under various input conditions. The American Journal of Clinical Nutrition. 2008; 87(6):1738-1742.
116. Holick M F. High prevalence of vitamin D inadequacy and implications for health. Mayo Clinic Proceedings. Mayo Clinic. 2006; 81(3):353-373.
117. Holscher C. Potential role of glucagon-like peptide-1 (GLP-1) in neuroprotection. CNS drugs. 2012; 26(10):871-882.
118. Hoist J J. The physiology of glucagon-like peptide 1. Physiological Reviews. 2007; 87(4):1409-1439.
119. Howard M A, Marczinski C A. Acute effects of a glucose energy drink on behavioral control. Experimental and Clinical Psychopharmacology. 2010; 18(6):553-561.
120. Huang M H, Schocken M, Block G, et al. Variation in nutrient intakes by ethnicity: results from the Study of Women's Health Across the Nation (SWAN). Menopause (New York, N.Y.). 2002; 9(5):309-319.
121. Ihrie R A, Shah J K, Harwell C C, et al. Persistent sonic hedgehog signaling in adult brain determines neural stem cell positional identity. Neuron. 2011; 71(2):250-262.
122. Inestrosa N C, Montecinos-Oliva C, Fuenzalida M. Wnt signaling: role in Alzheimer disease and schizophrenia. Journal of Neuroimmune Pharmacology: the official journal of the Society on Neurojmmune Pharmacology. 2012; 7(4):788-807.
123. Inomata S, Kadowaki S, Yamatani T, et al. Effect of 1 alpha (OH)-vitamin D3 on insulin secretion in diabetes mellitus. Bone and Mineral. 1986; 1(3):187-192.
124. Ito S, Ohtsuki S, Nezu Y, et al. 1alpha,25-Dihydroxyvitamin D3 enhances cerebral clearance of human amyloid-beta peptide(1-40) from mouse brain across the blood-brain barrier. Fluids and Barriers of the CNS. 2011; 8:20.
125. Ivanovic Z. Hypoxia or in situ normoxia: The stem cell paradigm. Journal of Cellular Physiology. 2009; 219(2):271-275.
126. Iwai T, Ito S, Tanimitsu K, et al. Glucagon-like peptide-1 inhibits LPS-induced IL-1beta production in cultured rat astrocytes. Neuroscience Research. 2006; 55(4):352-360.
127. Jaffe A B, Toran-Allerand C D, Greengard P, et al. Estrogen regulates metabolism of Alzheimer amyloid beta precursor protein. The Journal of Biological Chemistry. 1994; 269(18):13065-13068.
128. Jesmin S, Hattori Y, Sakuma I, et al. Estrogen deprivation and replacement modulate cerebral capillary density with vascular expression of angiogenic molecules in middle-aged female rats. Journal of Cerebral Blood Flow and Metabolism: official journal of the International Society of Cerebral Blood Flow and Metabolism. 2003; 23(2):181-189.
129. Johnston K L, Clifford M N, Morgan L M. Coffee acutely modifies gastrointestinal hormone secretion and glucose tolerance in humans: glycemic effects of chlorogenic acid and caffeine. The American Journal of Clinical Nutrition. 2003; 78(4):728-733.
130. Julien C, Tremblay C, Emond V, et al. Sirtuin 1 Reduction Parallels the Accumulation of Tau in Alzheimer Disease. Journal of Neuropathology and Experimental Neurology. 2009; 68(1):48-58.
131. Juntunen K S, Laaksonen D E, Autio K, et al. Structural differences between rye and wheat breads but not total fiber content may explain the lower postprandial insulin response to rye bread. The American Journal of Clinical Nutrition. 2003; 78(5):957-964.
132. Kancheva R, Hill M, Novak Z, et al. Neuroactive steroids in periphery and cerebrospinal fluid. Neuroscience. 2011; 191:22-27.
133. Karadsheh N, Kussie P, Linthicum D S. Inhibition of acetylcholinesterase by caffeine, anabasine, methyl pyrrolidine and their derivatives. Toxicology Letters. 1991; 55(3):335-342.
134. Kayisli U A, Aksu C A, Berkkanoglu M, et al. Estrogenicity of isoflavones on human endometrial stromal and glandular cells. The Journal of Clinical Endocrinology and Metabolism. 2002; 87(12):5539-5544.
135. Kim S M, Song J, Kim S, et al. Identification of peripheral inflammatory markers between normal control and Alzheimer's disease. BMC Neurology. 2011; 11:51.
136. Kim W, Kim M, Jho E H. Wnt/beta-catenin signalling: from plasma membrane to nucleus. The Biochemical Journal. 2013; 450(1):9-21.
137. King R A, Bursill D B. Plasma and urinary kinetics of the isoflavones daidzein and genistein after a single soy meal in humans. The American Journal of Clinical Nutrition. 1998; 67(5):867-872.
138. Kitazawa M, Cheng D, Tsukamoto M R, et al. Blocking IL-1 signaling rescues cognition, attenuates tau pathology, and restores neuronal beta-catenin pathway function in an Alzheimer's disease model. Journal of Immunology (Baltimore, Md.: 1950). 2011; 187(12):6539-6549.
139. Klebanoff M A, Levine R J, Dersimonian R, et al. Serum caffeine and paraxanthine as markers for reported caffeine intake in pregnancy. Annals of Epidemiology. 1998; 8(2):107-111.
140. Koga K, Nakamura M, Nakashima H, et al. Novel link between estrogen receptor alpha and hedgehog pathway in breast cancer. Anticancer Research. 2008; 28(2A):731-740.
141. Koole C, Pabreja K, Savage E E, et al. Recent advances in understanding GLP-1R (glucagon-like peptide-1 receptor) function. Biochemical Society Transactions. 2013; 41(1):172-179.
142. Koppelstaetter F, Poeppel T D, Siedentopf C M, et al. Caffeine and cognition in functional magnetic resonance imaging. Journal of Alzheimer's Disease: JAD. 2010; 20 Suppl 1:S71-84.
143. Koshiba T, Yokoshima S, Fukuyama T. Total Synthesis of (−)-Huperzine A. Organic Letters. 2009; 11(22):5354-5356.
144. Krause H P, Keup U, Puls W. Inhibition of disaccharide digestion in rat intestine by the alpha-glucosidase inhibitor acarbose (BAY g 5421). Digestion. 1982; 23(4):232-238.
145. Kuiper G G, Lemmen J G, Carlsson B, et al. Interaction of estrogenic chemicals and phytoestrogens with estrogen receptor beta. Endocrinology. 1998; 139(10):4252-4263.
146. Kuwabara T, Hsieh J, Muotri A, et al. Wnt-mediated activation of NeuroD1 and retro-elements during adult neurogenesis. Nature Neuroscience. 2009; 12(9):1097-1105.
147. L'Episcopo F, Tirolo C, Testa N, et al. Reactive astrocytes and Wnt/beta-catenin signaling link nigrostriatal injury to repair in 1-methyl-4-phenyl-1,2,3,6-tetrahydropyridine model of Parkinson's disease. Neurobiology of Disease. 2011; 41(2):508-527.
148. Lai K, Kaspar B K, Gage F H, et al. Sonic hedgehog regulates adult neural progenitor proliferation in vitro and in vivo. Nature Neuroscience. 2003; 6(1):21-27.
149. Lamprecht R, LeDoux J. Structural plasticity and memory. Nature Reviews. Neuroscience. 2004; 5(1):45-54.
150. Laske C, Stellos K, Stransky E, et al. Decreased plasma levels of granulocytecolony stimulating factor (G-CSF) in patients with early Alzheimer's disease. Journal of Alzheimer's Disease: JAD. 2009; 17(1):115-123.
151. Laughlin G A, McEvoy L K, Barrett-Connor E, et al. Fetuin-A, a New Vascular Biomarker of Cognitive Decline in Older Adults. Clinical Endocrinology. 2013.
152. Lechner D, Bajna E, Adlercreutz H, et al. Genistein and 17beta-estradiol, but not equol, regulate vitamin D synthesis in human colon and breast cancer cells. Anticancer Research. 2006; 26(4A):2597-2603.
153. Lee D Y, Na D L, Seo S W, et al. Association between cognitive impairment and bone mineral density in postmenopausal women. Menopause (New York, N.Y.). 2012; 19(6):636-641.
154. Lei H, Lu F, Dong H, et al. Genistein reverses free fatty acid-induced insulin resistance in HepG2 hepatocytes through targeting JNK. Journal of Huazhong University of Science and Technology. Medical Sciences. 2011; 31(2):185-189.
155. Leissring M A. The AbetaCs of Abeta-cleaving proteases. The Journal of Biological Chemistry. 2008; 283(44):29645-29649.
156. Lelo A, Birkett D J, Robson R A, et al. Comparative pharmacokinetics of caffeine and its primary demethylated metabolites paraxanthine, theobromine and theophylline in man. British Journal of Clinical Pharmacology. 1986; 22(2):177-182.
157. Leung R, Proitsi P, Simmons A, et al. Inflammatory proteins in plasma are associated with severity of Alzheimer's disease. PloS one. 2013; 8(6):e64971.
158. Li H L, Wang H H, Liu S J, et al. Phosphorylation of tau antagonizes apoptosis by stabilizing beta-catenin, a mechanism involved in Alzheimer's neurodegeneration. Proceedings of the National Academy of Sciences of the United States of America. 2007; 104(9):3591-3596.
159. Li R, Shen Y, Yang L B, et al. Estrogen enhances uptake of amyloid beta-protein by microglia derived from the human cortex. Journal of Neurochemistry. 2000; 75(4):1447-1454.
160. Li W, Li J, Hu Q. Determination of huperzine A in human plasma by liquid chromatography-electrospray tandem mass spectrometry: application to a bioequivalence study on Chinese volunteers. Biomedical chromatography: BMC. 2008; 22(4):354-360.
161. Li Y, Hansotia T, Yusta B, et al. Glucagon-like peptide-1 receptor signaling modulates beta cell apoptosis. The Journal of Biological Chemistry. 2003; 278(1):471-478.
162. Li Y, Luikart B W, Birnbaum S, et al. TrkB regulates hippocampal neurogenesis and governs sensitivity to antidepressive treatment. Neuron. 2008; 59(3):399-412.
163. Lie D C, Colamarino S A, Song H J, et al. Wnt signalling regulates adult hippocampal neurogenesis. Nature. 2005; 437(7063):1370-1375.
164. Liguori A, Hughes J R, Grass J A. Absorption and subjective effects of caffeine from coffee, cola and capsules. Pharmacology, Biochemistry, and Behavior. 1997; 58(3):721-726.
165. Liljeberg H G, Akerberg A K, Bjorck I M. Effect of the glycemic index and content of indigestible carbohydrates of cereal-based breakfast meals on glucose tolerance at lunch in healthy subjects. The American journal of Clinical Nutrition. 1999; 69(4):647-655.
166. Little J T, Walsh S, Aisen P S. An update on huperzine A as a treatment for Alzheimer's disease. Expert Opinion on Investigational Drugs. 2008; 17(2):209-215.
167. Liu A, Niswander L A. Bone morphogenetic protein signalling and vertebrate nervous system development. Nature reviews. Neuroscience. 2005; 6(12):945-954.
168. Liu R T, Zou L B, Fu J Y, et al. Promotion of rat brain-derived progenitor cell neurogenesis by liquiritigenin treatment: underlying mechanisms. Neuroscience Letters. 2010; 481(3):139-143.
169. Liu R T, Zou L B, Fu J Y, et al. Effects of liquiritigenin treatment on the learning and memory deficits induced by amyloid beta-peptide (25-35) in rats. Behavioural Brain Research. 2010; 210(1):24-31.
170. Liu R T, Zou L B, Lu Q J. Liquiritigenin inhibits Abeta(25-35)-induced neurotoxicity and secretion of Abeta(1-40) in rat hippocampal neurons. Acta Pharmacologica Sin ica. 2009; 30(7):899-906.
171. Liu Y, Liu F, Iqbal K, et al. Decreased glucose transporters correlate to abnormal hyperphosphorylation of tau in Alzheimer disease. FEBS letters. 2008; 582(2):359-364.
172. Llewellyn D J, Lang I A, Langa K M, et al. Vitamin D and cognitive impairment in the elderly U.S. population. The Journals of Gerontology. Series A, Biological sciences and medical sciences. 2011; 66(1):59-65.
173. Llewellyn D J, Lang I A, Langa K M, et al. Vitamin D and risk of cognitive decline in elderly persons. Archives of Internal Medicine. 2010; 170(13):1135-1141.
174. Llewellyn D J, Langa K M, Lang I A. Serum 25-hydroxyvitamin D concentration and cognitive impairment. Journal of Geriatric Psychiatry and Neurology. 2009; 22(3):188-195.
175. Loopstra-Masters R C, Fau-Liese A D, Liese Ad Fau-Haffner S M, Haffner Sm Fau-Wagenknecht L E, et al. Associations between the intake of caffeinated and decaffeinated coffee and measures of insulin sensitivity and beta cell function. Diabetologia 2001; 54:320-328.
176. Lord C, Buss C, Lupien S J, et al. Hippocampal volumes are larger in postmenopausal women using estrogen therapy compared to past users, never users and men: a possible window of opportunity effect. Neurobiology of Aging. 2008; 29(1):95-101.
177. Louvi A, Artavanis-Tsakonas S. Notch signalling in vertebrate neural development. Nature reviews. Neuroscience. 2006; 7(2):93-102.
178. LO PY YY, Wang W B, Liang C P, Li W B. Effects of huperzine A on [Ca2+]i level and expression of CaM, CaMPK II mRNA in hippocampal neurons of mice with vascular dementia. Clin J New Drugs Clin Rem. 2004; 23:73-76.
179. Lucas J J, Hernandez F, Gomez-Ramos P, et al. Decreased nuclear beta-catenin, tau hyperphosphorylation and neurodegeneration in GSK-3beta conditional transgenic mice. The EMBO Journal. 2001; 20(1-2):27-39.
180. Luchsinger J A. Type 2 diabetes, related conditions, in relation and dementia: an opportunity for prevention? Journal of Alzheimer's Disease: JAD. 2010; 20(3):723-736.
181. Luine V, Attalla S, Mohan G, et al. Dietary phytoestrogens enhance spatial memory and spine density in the hippocampus and prefrontal cortex of ovariectomized rats. Brain Research. 2006; 1126(1):183-187.
182. Luine V N. Estradiol increases choline acetyltransferase activity in specific basal forebrain nuclei and projection areas of female rats. Experimental Neurology. 1985; 89(2):484-490.
183. Ma J, Bellon M, Wishart J M, et al. Effect of the artificial sweetener, sucralose, on gastric emptying and incretin hormone release in healthy subjects. American Journal of Physiology. Gastrointestinal and Liver Physiology. 2009; 296(4):G735-739.
184. MacDonald B T, Tamai K, He X. Wnt/beta-catenin signaling: components, mechanisms, and diseases. Developmental Cell. 2009; 17(1):9-26.
185. Maestro B, Campion J, Davila N, et al. Stimulation by 1,25-dihydroxyvitamin D3 of insulin receptor expression and insulin responsiveness for glucose transport in U-937 human promonocytic cells. Endocrine journal. 2000; 47(4):383-391.
186. Maggiolini M, Picard D. The unfolding stories of GPR30, a new membrane-bound estrogen receptor. The Journal of Endocrinology. 2010; 204(2):105-114.
187. Maguschak K A, Ressler K J. A role for WNT/beta-catenin signaling in the neural mechanisms of behavior. Journal of Neuroimmune Pharmacology: the official journal of the Society on Neurolmmune Pharmacology. 2012; 7(4):763-773.
188. Maki P M. Hormone therapy and cognitive function: is there a critical period for benefit? Neuroscience. 2006; 138(3):1027-1030.
189. Maki P M, Resnick S M. Longitudinal effects of estrogen replacement therapy on PET cerebral blood flow and cognition. Neurobiology of Aging. 2000; 21(2):373-383.
190. Manthey D, Heck S, Engert S, et al. Estrogen induces a rapid secretion of amyloid beta precursor protein via the mitogen-activated protein kinase pathway. European Journal of Biochemistry/FEBS. 2001; 268(15):4285-4291.
191. Masoumi A, Goldenson B, Ghirmai S, et al. 1alpha,25-dihydroxyvitamin D3 interacts with curcuminoids to stimulate amyloid-beta clearance by macrophages of Alzheimer's disease patients. Journal of Alzheimer's Disease: JAD. 2009; 17(3):703-717.
192. Matsumoto Y, Otsuka F, Takano-Narazaki M, et al. Estrogen facilitates osteoblast differentiation by upregulating bone morphogenetic protein-4 signaling. Steroids. 2013; 78(5):513-520.
193. Mayeux R. Clinical practice. Early Alzheimer's disease. The New England Journal of Medicine. 2010; 362(23):2194-2201.
194. Mazumdar J, Dondeti V, Simon M C. Hypoxia-inducible factors in stem cells and cancer. Journal of Cellular and Molecular Medicine. 2009; 13(11-12):4319-4328.
195. McCarty M F. A chlorogenic acid-induced increase in GLP-1 production may mediate the impact of heavy coffee consumption on diabetes risk. Medical Hypotheses. 2005; 64(4):848-853.
196. McCarty M F. Down-regulation of microglial activation may represent a practical strategy for combating neurodegenerative disorders. Medical Hypotheses. 2006; 67(2):251-269.
197. Mersereau J E, Levy N, Staub R E, et al. Liquiritigenin is a plant-derived highly selective estrogen receptor beta agonist. Molecular and Cellular Endocrinology. 2008; 283(1-2):49-57.
198. Metzner J E, Frank T, Kunz I, et al. Study on the pharmacokinetics of synthetic genistein after multiple oral intake in post-menopausal women. Arzneimittel-Forschung. 2009; 59(10):513-520.
199. Miller D S, Bauer B, Hartz A M. Modulation of P-glycoprotein at the blood-brain barrier: opportunities to improve central nervous system pharmacotherapy. Pharmacological Reviews. 2008; 60(2):196-209.
200. Mira H, Andreu Z, Suh H, et al. Signaling through BMPR-IA regulates quiescence and long-term activity of neural stem cells in the adult hippocampus. Cell Stem Cell. 2010; 7(1):78-89.
201. Morris J C. The Clinical Dementia Rating (CDR): current version and scoring rules. Neurology. 1993; 43(11):2412-2414.
202. Morrison S J, Csete M, Groves A K, et al. Culture in reduced levels of oxygen promotes clonogenic sympathoadrenal differentiation by isolated neural crest stem cells. The Journal of Neuroscience: the official journal of the Society for Neuroscience. 2000; 20(19):7370-7376.
203. Moy G A, McNay E C. Caffeine prevents weight gain and cognitive impairment caused by a high-fat diet while elevating hippocampal BDNF. Physiology & Behavior. 2013; 109:69-74.
204. Nagele E, Han M, Demarshall C, et al. Diagnosis of Alzheimer's disease based on disease-specific autoantibody profiles in human sera. PloS one. 2011; 6(8):e23112.
205. Naveilhan P, Neveu I, Wion D, et al. 1,25-Dihydroxyvitamin D3, an inducer of glial cell line-derived neurotrophic factor. Neuroreport. 1996; 7(13):2171-2175.
206. Nebes R D, Pollock B G, Halligan E M, et al. Cognitive slowing associated with elevated serum anticholinergic activity in older individuals is decreased by caffeine use. The American Journal of Geriatric Psychiatry: official journal of the American Association for Geriatric Psychiatry. 2011; 19(2):169-175.
207. Neveu I, Naveilhan P, Baudet C, et al. 1,25-dihydroxyvitamin D3 regulates NT-3, NT-4 but not BDNF mRNA in astrocytes. Neuroreport. 1994; 6(1):124-126.
208. Neveu I, Naveilhan P, Jehan F, et al. 1,25-dihydroxyvitamin D3 regulates the synthesis of nerve growth factor in primary cultures of glial cells. Brain Research. Molecular Brain Research. 1994; 24(1-4):70-76.
209. Newton R, Broughton L J, Lind M J, et al. Plasma and salivary pharmacokinetics of caffeine in man. European Journal of Clinical Pharmacology. 1981; 21(1):45-52.
210. Norman A W. Minireview: vitamin D receptor: new assignments for an already busy receptor. Endocrinology. 2006; 147(12):5542-5548.
211. Osterlund M K, Gustafsson J A, Keller E, et al. Estrogen receptor beta (ERbeta) messenger ribonucleic acid (mRNA) expression within the human forebrain: distinct distribution pattern to ERalpha mRNA. The Journal of Clinical Endocrinology and Metabolism. 2000; 85(10):3840-3846.
212. Otani H, Otsuka F, Takeda M, et al. Regulation of GNRH production by estrogen and bone morphogenetic proteins in GT1-7 hypothalamic cells. The Journal of Endocrinology. 2009; 203(1):87-97.
213. Ottowitz W E, Siedlecki K L, Lindquist M A, et al. Evaluation of prefrontalhippocampal effective connectivity following 24 hours of estrogen infusion: an FDG-PET study. Psychoneuroendocrinology. 2008; 33(10):1419-1425.
214. Pan Y, Anthony M, Clarkson T B. Effect of estradiol and soy phytoestrogens on choline acetyltransferase and nerve growth factor mRNAs in the frontal cortex and hippocampus of female rats. Proceedings of the Society for Experimental Biology and Medicine. Society for Experimental Biology and Medicine (New York, N.Y.). 1999; 221(2):118-125.
215. Panchision D M. The role of oxygen in regulating neural stem cells in development and disease. Journal of cellular physiology. 2009; 220(3):562-568.
216. Park D C, Reuter-Lorenz P. The adaptive brain: aging and neurocognitive scaffolding. Annual Review of Psychology. 2009; 60:173-196.
217. Parker J, Hashmi O, Dutton D, et al. Levels of vitamin D and cardiometabolic disorders: systematic review and meta-analysis. Maturitas. 2010; 65(3):225-236.
218. Pei J J, Braak E, Braak H, et al. Distribution of active glycogen synthase kinase 3beta (GSK-3beta) in brains staged for Alzheimer disease neurofibrillary changes. Journal of Neuropathology and Experimental Neurology. 1999; 58(9):1010-1019.
219. Peng Y Fau-Lee D Y W, Lee Dy Fau-Jiang L, Jiang L Fau-Ma Z, et al. Huperzine A regulates amyloid precursor protein processing via protein kinase C and mitogen-activated protein kinase pathways in neuroblastoma SK-N-SH cells over-expressing wild type human amyloid precursor protein 695. (0306-4522 (Print)).
220. Perry T, Greig N H. Enhancing central nervous system endogenous GLP-1 receptor pathways for intervention in Alzheimer's disease. Current Alzheimer Research. 2005; 2(3):377-385.
221. Perry T, Lahiri D K, Chen D, et al. A novel neurotrophic property of glucagon-like peptide 1: a promoter of nerve growth factor-mediated differentiation in PC12 cells. The Journal of Pharmacology and Experimental Therapeutics. 2002; 300(3):958-966.
222. Perry T, Lahiri D K, Sambamurti K, et al. Glucagon-like peptide-1 decreases endogenous amyloid-beta peptide (Abeta) levels and protects hippocampal neurons from death induced by Abeta and iron. Journal of Neuroscience Research. 2003; 72(5):603-612.
223. Perry V H. The influence of systemic inflammation on inflammation in the brain: implications for chronic neurodegenerative disease. Brain, Behavior, and Immunity. 2004; 18(5):407-413.
224. Pistollato F, Chen H L, Rood B R, et al. Hypoxia and HIF1alpha repress the differentiative effects of BMPs in high-grade glioma. Stem Cells (Dayton, Ohio). 2009; 27(1):7-17.
225. Plassman B L, Langa K M, Fisher G G, et al. Prevalence of dementia in the United States: the aging, demographics, and memory study. Neuroepidemiology. 2007; 29(1-2):125-132.
226. Purro S A, Dickins E M, Salinas P C. The secreted Wnt antagonist Dickkopf-1 is required for amyloid beta-mediated synaptic loss. The Journal of Neuroscience: the official journal of the Society for Neuroscience. 2012; 32(10):3492-3498.
227. Qosa H, Abuznait A H, Hill R A, et al. Enhanced brain amyloid-beta clearance by rifampicin and caffeine as a possible protective mechanism against Alzheimer's disease. Journal of Alzheimer's disease: JAD. 2012; 31(1):151-165.
228. Ray S, Britschgi M, Herbert C, et al. Classification and prediction of clinical Alzheimer's diagnosis based on plasma signaling proteins. Nature Medicine. 2007; 13(11):1359-1362.
229. Raz N, Rodrigue K M, Kennedy K M, et al. Hormone replacement therapy and age-related brain shrinkage: regional effects. Neuroreport. 2004; 15(16):2531-2534.
230. Reyes A E, Chacon M A, Dinamarca M C, et al. Acetylcholinesterase-Abeta complexes are more toxic than Abeta fibrils in rat hippocampus: effect on rat beta-amyloid aggregation, laminin expression, reactive astrocytosis, and neuronal cell loss. The American Journal of Pathology. 2004; 164(6):2163-2174.
231. Roberts R O, Geda Y E, Knopman D S, et al. Association of C-reactive protein with mild cognitive impairment. Alzheimer's & Dementia: the journal of the Alzheimer's Association. 2009; 5(5):398-405.
232. Ronis M J, Chen Y, Badeaux J, et al. Dietary soy protein isolate attenuates metabolic syndrome in rats via effects on PPAR, LXR, and SREBP signaling. The Journal of Nutrition. 2009; 139(8):1431-1438.
233. Rosario E R, Chang L, Head E H, et al. Brain levels of sex steroid hormones in men and women during normal aging and in Alzheimer's disease. Neurobiology of Aging. 2011; 32(4):604-613.
234. Rosi M C, Luccarini I, Grossi C, et al. Increased Dickkopf-1 expression in transgenic mouse models of neurodegenerative disease. Journal of Neurochemistry. 2010; 112(6):1539-1551.
235. Ross A C, Manson J E, Abrams S A, et al. The 2011 report on dietary reference intakes for calcium and vitamin D from the Institute of Medicine: what clinicians need to know. The Journal of Clinical Endocrinology and Metabolism. 2011; 96(1):53-58.
236. Rossini M, Gatti D, Adami S. Involvement of WNT/beta-catenin signaling in the treatment of osteoporosis. Calcified Tissue International. 2013; 93(2):121-132.
237. Rothman S M, Griffioen K J, Wan R, et al. Brain-derived neurotrophic factor as a regulator of systemic and brain energy metabolism and cardiovascular health. Annals of the New York Academy of Sciences. 2012; 1264(1):49-63.
238. Roy N S, Wang S, Jiang L, et al. In vitro neurogenesis by progenitor cells isolated from the adult human hippocampus. Nature Medicine. 2000; 6(3):271-277.
239. Rozovsky I, Finch C E, Morgan T E. Age-related activation of microglia and astrocytes: in vitro studies show persistent phenotypes of aging, increased proliferation, and resistance to down-regulation. Neurobiology of Aging. 1998; 19(1):97-103.
240. Salcedo I, Tweedie D, Li Y, et al. Neuroprotective and neurotrophic actions of glucagon-like peptide-1: an emerging opportunity to treat neurodegenerative and cerebrovascular disorders. British Journal of Pharmacology. 2012; 166(5):1586-1599.
241. Sallaberry C, Nunes F, Costa M S, et al. Chronic caffeine prevents changes in inhibitory avoidance memory and hippocampal BDNF immunocontent in middleaged rats. Neuropharmacology. 2013; 64:153-159.
242. Sanchez-Ramos J, Song S, Sava V, et al. Granulocyte colony stimulating factor decreases brain amyloid burden and reverses cognitive impairment in Alzheimer's mice. Neuroscience. 2009; 163(1):55-72.
243. Santos C, Costa J, Santos J, et al. Caffeine intake and dementia: systematic review and meta-analysis. Journal of Alzheimer's disease: JAD. 2010; 20 Suppl 1:S187-204.
244. Santos C, Lunet N, Azevedo A, et al. Caffeine intake is associated with a lower risk of cognitive decline: a cohort study from Portugal. Journal of Alzheimer's Disease: JAD. 2010; 20 Suppl 1:S175-185.
245. Sato A, Sato Y, Uchida S. Activation of the intracerebral cholinergic nerve fibers originating in the basal forebrain increases regional cerebral blood flow in the rat's cortex and hippocampus. Neuroscience letters. 2004; 361(1-3):90-93.
246. Sato H, Genet C, Strehle A, et al. Anti-hyperglycemic activity of a TGR5 agonist isolated from Olea europaea. Biochemical and Biophysical Research Communications. 2007; 362(4):793-798.
247. Scali C, Caraci F, Gianfriddo M, et al. Inhibition of Wnt signaling, modulation of Tau phosphorylation and induction of neuronal cell death by DKK1. Neurobiology of disease. 2006; 24(2):254-265.
248. Scott E L, Brann D W. Estrogen regulation of Dkk1 and Wnt/beta-Catenin signaling in neurodegenerative disease. Brain Research. 2013; 1514:63-74.
249. Scott E L, Zhang O G, Han D, et al. Long-term estrogen deprivation leads to elevation of Dickkopf-1 and dysregulation of Wnt/beta-Catenin signaling in hippocampal CA1 neurons. Steroids. 2013; 78(6):624-632.
250. Seib D R, Corsini N S, Ellwanger K, et al. Loss of Dickkopf-1 restores neurogenesis in old age and counteracts cognitive decline. Cell Stem Cell. 2013; 12(2):204-214.
251. Seifert-Held T, Pekar T, Gattringer T, et al. Circulating Dickkopf-1 in acute ischemic stroke and clinically stable cerebrovascular disease. Atherosclerosis. 2011; 218(1):233-237.
252. Setchell K D, Brown N M, Lydeking-Olsen E. The clinical importance of the metabolite equol—a clue to the effectiveness of soy and its isoflavones. The Journal of Nutrition. 2002; 132(12):3577-3584.
253. Setchell K D, Cole S J. Method of defining equol-producer status and its frequency among vegetarians. The Journal of Nutrition. 2006; 136(8):2188-2193.
254. Shang Y Z, Ye J W, Tang X C. Improving effects of huperzine A on abnormal lipid peroxidation and superoxide dismutase in aged rats. Zhongguo Yao, Li Xue Bao Acta Pharmacologica Sinica. 1999; 20(9):824-828.
255. Shankar G M, Li S, Mehta T H, et al. Amyloid-beta protein dimers isolated directly from Alzheimer's brains impair synaptic plasticity and memory. Nature Medicine. 2008; 14(8):837-842.
256. Sharma P K, Thakur M K. Expression of estrogen receptor (ER) alpha and beta in mouse cerebral cortex: effect of age, sex and gonadal steroids. Neurobiology of Aging. 2006; 27(6):880-887.
257. Shen Q, Wang Y, Kokovay E, et al. Adult SVZ stem cells lie in a vascular niche: a quantitative analysis of niche cell-cell interactions. Cell Stem Cell. 2008; 3(3):289-300.
258. Sherwin B B, Henry J F. Brain aging modulates the neuroprotective effects of estrogen on selective aspects of cognition in women: a critical review. Frontiers in Neuroendocrinology. 2008; 29(1):88-113.
259. Shi M, Caudle W M, Zhang J. Biomarker discovery in neurodegenerative diseases: A proteomic approach. Neurobiology of Disease. 2009; 35(2):157-164.
260. Shimazu K, Zhao M, Sakata K, et al. NT-3 facilitates hippocampal plasticity and learning and memory by regulating neurogenesis. Learning & Memory (Cold Spring Harbor, N.Y.). 2006; 13(3):307-315.
261. Shu X O, Zheng Y, Cai H, et al. Soy food intake and breast cancer survival. JAMA: the Journal of the American Medical Association. 2009; 302(22):2437-2443.
262. Simons S B, Caruana D A, Zhao M, et al. Caffeine-induced synaptic potentiation in hippocampal CA2 neurons. Nature Neuroscience. 2012; 15(1):23-25.
263. Singh M, Meyer E M, Millard W J, et al. Ovarian steroid deprivation results in a reversible learning impairment and compromised cholinergic function in female Sprague-Dawley rats. Brain Research. 1994; 644(2):305-312.
264. Singh M, Meyer E M, Simpkins J W. The effect of ovariectomy and estradiol replacement on brain-derived neurotrophic factor messenger ribonucleic acid expression in cortical and hippocampal brain regions of female Sprague-Dawley rats. Endocrinology. 1995; 136(5):2320-2324.
265. Singh M N, Hemant K S Y, Ram M, et al. Microencapsulation: A promising technique for controlled drug delivery. Research in Pharmaceutical Sciences. 2010; 5(2):65-77.
266. Smiley D A, Khalil R A. Estrogenic compounds, estrogen receptors and vascular cell signaling in the aging blood vessels. Current Medicinal Chemistry. 2009; 16(15):1863-1887.
267. Smith E R, Nilforooshan R, Weaving G, et al. Plasma fetuin-A is associated with the severity of cognitive impairment in mild-to-moderate Alzheimer's disease. Journal of Alzheimer's disease: JAD. 2011; 24(2):327-333.
268. Society N A M. The role of soy isoflavones in menopausal health: report of The North American Menopause Society/Wulf H. Utian Translational Science Symposium in Chicago, Ill. (October 2010). Menopause (New York, N.Y.). 2011; 18(7):732-753.
269. Sohrabji F, Miranda R C, Toran-Allerand C D. Identification of a putative estrogen response element in the gene encoding brain-derived neurotrophic factor. Proceedings of the National Academy of Sciences of the United States of America. 1995; 92(24):11110-11114.
270. Song F, Smythe G A, Sachdev P S, et al. Plasma apolipoprotein levels are associated with cognitive status and decline in a community cohort of older individuals. PloS one. 2012; 7(6):e34078.
271. Song H, Stevens C F, Gage F H. Astroglia induce neurogenesis from adult neural stem cells. Nature. 2002; 417(6884):39-44.
272. Sonnenberg J, Luine V N, Krey L C, et al. 1,25-Dihydroxyvitamin D3 treatment results in increased choline acetyltransferase activity in specific brain nuclei. Endocrinology. 1986; 118(4):1433-1439.
273. Stewart A, Wong K, Cachat J, et al. Neurosteroid vitamin D system as a nontraditional drug target in neuropsychopharmacology. Behavioural Pharmacology. 2010; 21(5-6):420-426.
274. Stirone C, Boroujerdi A, Duckles S P, et al. Estrogen receptor activation of phosphoinositide-3 kinase, akt, and nitric oxide signaling in cerebral blood vessels: rapid and long-term effects. Molecular Pharmacology. 2005; 67(1):105-113.
275. Stoppelkamp S, Bell H S, Palacios-Filardo J, et al. In vitro modelling of Alzheimer's disease: degeneration and cell death induced by viral delivery of amyloid and tau. Experimental Neurology. 2011; 229(2):226-237.
276. Sun Y, Yin X S, Guo H, et al. Elevated osteopontin levels in mild cognitive impairment and Alzheimer's disease. Mediators of Inflammation. Hindawi 2013. ID 615745
277. Sung C C, Liao M T, Lu K C, et al. Role of vitamin D in insulin resistance. Journal of Biomedicine & Biotechnology. 2012; 2012:634195
278. Sutherland M K, Somerville M J, Yoong L K, et al. Reduction of vitamin D hormone receptor mRNA levels in Alzheimer as compared to Huntington hippocampus: correlation with calbindin-28k mRNA levels. Brain Research. Molecular Brain Research. 1992; 13(3):239-250.
279. Tague S E, Smith P G. Vitamin D receptor and enzyme expression in dorsal root ganglia of adult female rats: modulation by ovarian hormones. Journal of Chemical Neuroanatomy. 2011; 41(1):1-12.
280. Tai K, Need A G, Horowitz M, et al. Vitamin D, glucose, insulin, and insulin sensitivity. Nutrition (Burbank, Los Angeles County, Calif.). 2008; 24(3):279-285.
281. Takiishi T, Gysemans C, Bouillon R, et al. Vitamin D and diabetes. Endocrinology and Metabolism Clinics of North America. 2010; 39(2):419-446.
282. Takimoto C H, Goetz A, Llorens V, et al. Phase I pharmacokinetic and pharmacodynamic analysis of unconjugated soy isoflavones administered to individuals with cancer. Cancer Epidemiology, Biomarkers & Prevention: a publication of the American Association for Cancer Research, cosponsored by the American Society of Preventive Oncology. 2003; 12(11 Pt 1):1213-1221.
283. Thomas P, Pang Y, Filardo E J, et al. Identity of an estrogen membrane receptor coupled to a G protein in human breast cancer cells. Endocrinology. 2005; 146(2):624-632.
284. Thum A, Hupe-Sodmann K, Goke R, et al. Endoproteolysis by isolated membrane peptidases reveal metabolic stability of glucagon-like peptide-1 analogs, exendins-3 and -4. Experimental and Clinical Endocrinology & Diabetes: official journal, German Society of Endocrinology and German Diabetes Association. 2002; 110(3):113-118.
285. Traiffort E, Charytoniuk D A, Faure H, et al. Regional distribution of Sonic Hedgehog, patched, and smoothened mRNA in the adult rat brain. Journal of Neurochemistry. 1998; 70(3):1327-1330.
286. Tsutsumi S, Zhang X, Takata K, et al. Differential regulation of the inducible nitric oxide synthase gene by estrogen receptors 1 and 2. The Journal of Endocrinology. 2008; 199(2):267-273.
287. Tudhope S R, Bellamy J A, Ball A, et al. Development of a Large-Scale Synthetic Route to Manufacture (−)-Huperzine A. Organic Process Research & Development. 2012; 16(4):635-642.
288. Tun M K H S. The pharmacology and therapeutic potential of (−)-huperzine A. J Exp Pharmacol. 2012; 4:113-123.
289. Unfer V, Casini M L, Costabile L, et al. Endometrial effects of long-term treatment with phytoestrogens: a randomized, double-blind, placebo-controlled study. Fertility and Sterility. 2004; 82(1):145-148, quiz 265.
290. Uranga R M, Bruce-Keller A J, Morrison C D, et al. Intersection between metabolic dysfunction, high fat diet consumption, and brain aging. Journal of Neurochemistry. 2010; 114(2):344-361.
291. Varea O, Arevalo M A, Garrido J J, et al. Interaction of estrogen receptors with insulin-like growth factor-I and Wnt signaling in the nervous system. Steroids. 2010; 75(8-9):565-569.
292. Varela-Nallar L, Alfaro I E, Serrano F G, et al. Wingless-type family member 5A (Wnt-5a) stimulates synaptic differentiation and function of glutamatergic synapses. Proceedings of the National Academy of Sciences of the United States of America. 2010; 107(49):21164-21169.
293. Verheus M, van Gils C H, Keinan-Boker L, et al. Plasma phytoestrogens and subsequent breast cancer risk. Journal of Clinical Oncology: official journal of the American Society of Clinical Oncology. 2007; 25(6):648-655.
294. Vila-Luna S, Cabrera-Isidoro S, Vila-Luna L, et al. Chronic caffeine consumption prevents cognitive decline from young to middle age in rats, and is associated with increased length, branching, and spine density of basal dendrites in CA1 hippocampal neurons. Neuroscience. 2012; 202:384-395.
295. Voytko M L, Tinkler G P, Browne C, et al. Neuroprotective effects of estrogen therapy for cognitive and neurobiological profiles of monkey models of menopause. American Journal of Primatology. 2009; 71(9):794-801.
296. Wandosell F, Varea O, Arevalo M A, et al. Oestradiol regulates beta-cateninmediated transcription in neurones. Journal of Neuroendocrinology. 2012; 24(1):191-194.
297. Wang C, Liu F, Liu Y Y, et al. Identification and characterization of neuroblasts in the subventricular zone and rostral migratory stream of the adult human brain. Cell Research. 2011; 21(11):1534-1550.
298. Wang C Y, Zheng W, Wang T, et al. Huperzine A activates Wnt/beta-catenin signaling and enhances the nonamyloidogenic pathway in an Alzheimer transgenic mouse model. Neuropsychopharmacology: official publication of the American College of Neuropsychopharmacology. 2011; 36(5):1073-1089.
299. Wang R, Yan H, Tang X C. Progress in studies of huperzine A, a natural cholinesterase inhibitor from Chinese herbal medicine. Acta Pharmacologica Sinica. 2006; 27(1):1-26.
300. Wang Y, Chiang Y H, Su T P, et al. Vitamin D(3) attenuates cortical infarction induced by middle cerebral arterial ligation in rats. Neuropharmacology. 2000; 39(5):873-880.
301. Wergeland S, Torkildsen O, Myhr K M, et al. Dietary vitamin D3 supplements reduce demyelination in the cuprizone model. PloS one. 2011; 6(10):e26262.
302. Wilkins C H, Birge S J, Sheline Y I, et al. Vitamin D deficiency is associated with worse cognitive performance and lower bone density in older African Americans. Journal of the National Medical Association. 2009; 101(4):349-354.
303. Wolever™, Bentum-Williams A, Jenkins D J. Physiological modulation of plasma free fatty acid concentrations by diet. Metabolic implications in nondiabetic subjects. Diabetes Care. 1995; 18(7):962-970.
304. Woolley C S. Acute effects of estrogen on neuronal physiology. Annual Review of Pharmacology and Toxicology. 2007; 47:657-680.
305. Wostyn P, Van Dam D, Audenaert K, et al. Increased Cerebrospinal Fluid Production as a Possible Mechanism Underlying Caffeine's Protective Effect against Alzheimer's Disease. International Journal of Alzheimer's Disease. 2011; 2011:617420.
306. Wu T, Zhao B R, Bound M J, et al. Effects of different sweet preloads on incretin hormone secretion, gastric emptying, and postprandial glycemia in healthy humans. The American Journal of Clinical Nutrition. 2012; 95(1):78-83.
307. Xiao X Q, Zhang H Y, Tang X C. Huperzine A attenuates amyloid beta-peptide fragment 25-35-induced apoptosis in rat cortical neurons via inhibiting reactive oxygen species formation and caspase-3 activation. Journal of Neuroscience Research. 2002; 67(1):30-36.
308. Xu H, Huang W, Wang Y, et al. The function of BMP4 during neurogenesis in the adult hippocampus in Alzheimer's disease. Ageing Research Reviews. 2013; 12(1):157-164.
309. Xu S L, Bi C W, Choi R C, et al. Flavonoids induce the synthesis and secretion of neurotrophic factors in cultured rat astrocytes: a signaling response mediated by estrogen receptor. Evidence-based Complementary and Alternative Medicine: eCAM. 2013; 2013:127075.
310. Yabe D, Seino Y. Two incretin hormones GLP-1 and GIP: comparison of their actions in insulin secretion and beta cell preservation. Progress in Biophysics and Molecular Biology. 2011; 107(2):248-256.
311. Yamada M, Lamping K G, Duttaroy A, et al. Cholinergic dilation of cerebral blood vessels is abolished in M(5) muscarinic acetylcholine receptor knockout mice. Proceedings of the National Academy of Sciences of the United States of America. 2001; 98(24):14096-14101.
312. Yang G, Shu X O, Li H, et al. Prospective cohort study of soy food intake and colorectal cancer risk in women. The American Journal of Clinical Nutrition. 2009; 89(2):577-583.
313. Yoon K, Gaiano N. Notch signaling in the mammalian central nervous system: insights from mouse mutants. Nature Neuroscience. 2005; 8(6):709-715.
314. Yoshii A, Constantine-Paton M. Postsynaptic BDNF-TrkB signaling in synapse maturation, plasticity, and disease. Developmental Neurobiology. 2010; 70(5):304-322.
315. Zeitlin R, Patel S, Burgess S, et al. Caffeine induces beneficial changes in PKA signaling and JNK and ERK activities in the striatum and cortex of Alzheimer's transgenic mice. Brain Research. 2011; 1417:127-136.
316. Zhang H Y, Liang Y Q, Tang X C, et al. Stereoselectivities of enantiomers of huperzine A in protection against beta-amyloid(25-35)-induced injury in PC12 and NG108-15 cells and cholinesterase inhibition in mice. Neuroscience Letters. 2002; 317(3):143-146.
317. Zhang H Y, Yan H, Tang X C. Huperzine A enhances the level of secretory amyloid precursor protein and protein kinase C-alpha in intracerebroventricular beta-amyloid-(1-40) infused rats and human embryonic kidney 293 Swedish mutant cells. Neuroscience Letters. 2004; 360(1-2):21-24.
318. Zhang J M, Hu G Y. Huperzine A, a nootropic alkaloid, inhibits N-methyl-Daspartate-induced current in rat dissociated hippocampal neurons. Neuroscience. 2001; 105(3):663-669.
319. Zhang Q G, Wang R, Khan M, et al. Role of Dickkopf-1, an antagonist of the Wnt/beta-catenin signaling pathway, in estrogen-induced neuroprotection and attenuation of tau phosphorylation. The Journal of Neuroscience: the official journal of the Society for Neuroscience. 2008; 28(34):8430-8441.
320. Zhang S, Huang Y, Zhu Y C, et al. Estrogen stimulates release of secreted amyloid precursor protein from primary rat cortical neurons via protein kinase C pathway. Acta Pharmacologica Sinica. 2005; 26(2):171-176.
321. Zhang Z Fau-Wang X, Wang X Fau-Chen Q, Chen Q Fau-Shu L, et al. [Clinical efficacy and safety of huperzine Alpha in treatment of mild to moderate Alzheimer disease, a placebo-controlled, double-blind, randomized trial]. (0376-2491.
322. Zhao L, O'Neill K, Diaz Brinton R. Selective estrogen receptor modulators (SERMs) for the brain: current status and remaining challenges for developing NeuroSERMs. Brain Research. Brain research reviews. 2005; 49(3):472-493.
323. Zhao T, Zhang C P, Liu Z H, et al. Hypoxia-driven proliferation of embryonic neural stem/progenitor cells—role of hypoxia-inducible transcription factor-1 alpha. The FEBS journal. 2008; 275(8):1824-1834.
324. Zhou C J, Borello U, Rubenstein J L, et al. Neuronal production and precursor proliferation defects in the neocortex of mice with loss of function in the canonical Wnt signaling pathway. Neuroscience. 2006; 142(4):1119-1131.
325. Zhou J, Fu Y, Tang X C. Huperzine A and donepezil protect rat pheochromocytoma cells against oxygen-glucose deprivation. Neuroscience Letters. 2001; 306(1-2):53-56.
326. Zhou R, Deng J, Zhang M, et al. Association between bone mineral density and the risk of Alzheimer's disease. Journal of Alzheimer's disease: JAD. 2011; 24(1):101-108.
327. Zhu Y, Zhou R, Yang R, et al. Abnormal neurogenesis in the dentate gyrus of adult mice lacking 1,25-dihydroxy vitamin D3 (1,25-(OH)2 D3). Hippocampus. 2012; 22(3):421-433.
328. Zipfel G J, Babcock D J, Lee J M, et al. Neuronal apoptosis after CNS injury: the roles of glutamate and calcium. Journal of neurotrauma. 2000; 17(10):857-869.
329. Zlokovic B V. The blood-brain barrier in health and chronic neurodegenerative disorders. Neuron. 2008; 57(2):178-201.
330. U.S. Pat. No. 6,524,616.

1.-102. (canceled)

103. A composition comprising Huperzine A or a derivative or analog thereof; one or more estrogens and phytoestrogens; and a vitamin D.

104. The composition of claim 103, wherein the phytoestrogen is a soy phytoestrogen selected from the group consisting of an isoflavone, a coumestan, a lignan, synthetic analogs and derivatives thereof, and combinations thereof.

105. The composition of claim 103, wherein the vitamin D is selected from the group consisting of calcitriol, doxercalciferol, paricalcitol, cholecalciferol (vitamin D3), ergocalciferol (vitamin D2), analogs and derivatives thereof, vitamin D receptor agonists and modulators, and combinations thereof.

106. The composition of claim 103, further comprising one or more additives selected from the group consisting of coffee, xanthine alkaloids, chlorogenic acid, sweeteners, and combinations thereof, and wherein the xanthene alkoloid is selected from the group consisting of caffeine, theobromine, paraxanthine, and combinations thereof, and the sweetener is selected from the group consisting sucromalt, tagatose, isomalt, sucralose, acesulfame potassium, analogs and derivatives thereof, and combinations thereof.

107. The composition of claim 103, wherein the composition comprises from about 0.01 mg to about 150 mg of Huperzine A or an analog or derivative thereof.

108. The composition of claim 103, wherein the composition comprises from about 0.01 mg to about 1000 mg of at least one phytoestrogen.

109. The composition of claim 103, wherein the composition comprises from about 200 iu to about 5000 iu of vitamin D, an analog thereof, or a vitamin D receptor agonist and modulator.

110. The composition of claim 106, wherein the composition comprises from about 10 mg to about 100 mg of the xanthine alkaloid.

111. The composition of claim 106, wherein the composition comprises from about 10 g to about 100 g of the sweetener.

112. The composition of claim 103, wherein the composition comprises Huperzine A, soy isoflavone, and vitamin D.

113. The composition of claim 106, wherein the composition comprises from about 40 mcg to about 400 mcg of Huperzine A, about 110 mg of soy isoflavone, about 1200 iu of vitamin D, about 75 mg of caffeine, and about 75 g of sucromalt.

114. The composition of claim 103, wherein the composition is a nutraceutical composition.

115. The composition of claim 103, wherein the composition is a pharmaceutical composition comprising genistein or daidzein, vitamin D, Huperzine A, and estrogen.

116. The composition of claim 115, wherein Huperzine A, genistein or daidzein, and vitamin D are synthetic compounds and estrogen is 17-beta estradiol.

117. The composition of claim 103, wherein the composition is formulated for immediate release, extended release, or timed release.

118. The composition of claim 103, wherein the composition is formulated in the form of a tablet, a capsule, a powder, an emulsion, a suspension, a syrup, a solution, a gel, or a patch.

119. A pharmaceutical composition comprising the composition of claim 103 and a pharmaceutically acceptable carrier.

120. A method of promoting healthy brain aging of a subject, wherein the method comprises administering an effective amount of the pharmaceutical composition of claim 119 to the subject in need thereof.

121. A method of promoting, stimulating or inducing neurogenisis of cells, wherein neurogenesis comprises production of neurotrophins and/or neurotransmitters, the method comprising administering an effective amount of the pharmaceutical composition of claim 119 to a subject in need thereof, and wherein the neurotrophins are selected from the group consisting of brain derived neurotrophic factor, nerve growth factor, Sonic hedgehog, Notch, brain morphogenetic protein, and combinations thereof, and the neurotransmitters are selected from the group consisting of serotonin, glutamate, acetylcholine, and combinations thereof.

122. The method of claim 121, wherein the cells are neural stem cells or neural progenitor cells.

123. The method of stimulating and/or activating the wnt/beta catenin pathway by a combination of stimulating the synthesis of beta catenin and the binding of beta catenin to its receptor, and/or by inhibiting natural antagonists of the wnt/beta catenin pathway, wherein the method comprises administering an effective amount of the pharmaceutical composition of claim 119 to a subject in need thereof.

124. The method of promoting the wnt/beta catenin pathway according to claim 123, wherein the natural antagonist is selected from Dkk-1 and GSK-3 beta.

125. A method of inhibiting apoptosis of neuronal cells wherein the method comprises administering an effective amount of the pharmaceutical composition of claim 119 to a subject in need thereof to promote the expression of Bcl-2 and/or to inhibit the expression of P53 or Bax.

126. A method of providing neuroprotection of the brain, the method comprising administering an effective amount of the pharmaceutical composition of claim 119 to a subject in need thereof.

127. The method of claim 126, wherein the method inhibits the formation and/or accumulation of beta amyloid to neuronal cells expressing amyloid precursor protein (APP), and/or stimulates cleavage of APP via the alpha secretase pathway, and/or inhibits the beta and gamma secretase pathways.

128. A method of inhibiting the formation of neurofibrillary tangles and deacetylating of tau protein, wherein the method comprises administering an effective amount of the pharmaceutical composition of claim 119 to a subject in need thereof.

129. A method of inducing the expression of sirtuin genes, wherein the sirtuin genes comprise a SIRT1 gene, and wherein the method comprises administering an effective amount of the pharmaceutical composition of claim 119 to a subject in need thereof.

130. A method of promoting an increase in efflux of soluble non-amyloidogenic beta amyloid metabolites from neuronal cells into the blood stream, wherein the method comprises administering an effective amount of the pharmaceutical composition of claim 119 to a subject in need thereof.

131. A method of protecting and/or maintaining the integrity of the blood brain barrier (BBB) and/or facilitating glucose transport across the BBB, wherein the method comprises administering an effective amount of the pharmaceutical composition of claim 119 to a subject in need thereof.

132. A method of inhibiting inflammation in a subject, wherein the method comprises administering an effective amount of the pharmaceutical composition of claim 119 to the subject in need thereof such that secretion of inflammatory cytokines is inhibited and/or cytokine levels in the brain are reduced.

133. A method of inhibiting activation of microglial cells, wherein the method comprises administering an effective amount of the pharmaceutical composition of claim 119 to a subject in need thereof.

134. A method of modulating, treating, inhibiting, retarding, reducing and/or preventing oxidative stress and/or accumulation of oxygen radicals in the brain, wherein the method comprises administering an effective amount of the pharmaceutical composition of claim 119 to a subject in need thereof.

135. A method of enhancing brain insulin metabolism by stimulating synthesis of insulin and/or promoting insulin sensitivity in the brain of a subject and/or by inhibiting insulin resistance in neuronal cells, wherein the method comprises administering an effective amount of the pharmaceutical composition of claim 119 to the subject in need thereof.

136. A method of enhancing cerebral blood flow and/or supply of oxygen to the brain of a subject, wherein the method comprises administering an effective amount of the pharmaceutical composition of claim 119 to the subject in need thereof.

137. A method of stimulating neurotransmitters and their respective neurotransmission, wherein the neurotransmitters are selected from a group consisting of serotonin, glutamate, acetylcholine and a combination thereof, and wherein the method comprises administering an effective amount of the pharmaceutical composition of claim 119 to a subject in need thereof.

138. A method of enhancing acetylcholine synthesis in a subject by stimulating acetylcholine transferase activity and/or inhibiting cholinesterase activity, wherein the method comprises administering an effective amount of the pharmaceutical composition of claim 119 to the subject in need thereof.

139. A method of inhibiting glutamate toxicity, wherein the method comprises administering an effective amount of the pharmaceutical composition of claim 119 to a subject in need thereof.

140. A method of modulating N-methyl-D-aspartate (NMDA) receptors, wherein the method comprises administering an effective amount of the pharmaceutical composition of claim 119 to a subject in need thereof.

141. A method of preventing, inhibiting, retarding or treating neuronal degeneration and/or a decline in cognitive function in a subject at increased risk of impaired cognitive function, executive function or memory disorder, wherein the method comprises administering an effective amount of the pharmaceutical composition of claim 119 to the subject in need thereof.

142. A method of alleviating the symptoms of a subject diagnosed with mild cognitive impairment and/or Alzheimer's Disease, wherein the method comprises administering an effective amount of the pharmaceutical composition of claim 119 to the subject in need thereof.

143. A method of preventing, retarding or inhibiting mild cognitive impairment and/or Alzheimer's disease to a subject at risk of developing or diagnosed with mild cognitive impairment and/or Alzheimer's disease, wherein the method comprises administering an effective amount of the pharmaceutical composition of claim 119 to the subject in need thereof.

144. A method of preventing, retarding or treating dementia and/or cognitive impairment, wherein the method comprises administering an effective amount of the pharmaceutical composition of claim 119 to a subject in need thereof.

145. A method of treating a subject with hypercholesteremia, metabolic syndrome, type II diabetes, obesity, osteopenia, osteoporosis, hypertension, and post menopausal women on hormone replacement therapy wherein the method comprises administering an effective amount of the pharmaceutical composition of claim 119 to the subject in need thereof as adjunctive therapy with other drugs, wherein the other drugs are for treating a primary disease in the subject.

146. A method of individualizing the dosage of the pharmaceutical composition of claim 119 to promote brain health and treatment of cognitive dysfunction and age related dementia in a subject, wherein the method comprises administering an individual effective amount of the composition of claim 119 to the subject in need thereof.

147. A method of measuring and monitoring absorption of bioactive levels of the components of the pharmaceutical composition of claim 119, wherein the method comprises
(a) administering an effective amount of the composition of claim 119 to a subject in need thereof;
(b) measuring and monitoring the absorption of bioactive levels of the components; and
(c) determining whether an optimal level or range of each component has been reached for maintaining a healthy brain or for the treat of the symptoms of mild cognitive impairment, dementia, or Alzheimer's disease.

148. A method of measuring and monitoring the bioactive brain health protective efficacy of the pharmaceutical composition of claim 119, the method comprising
(a) assaying brain specific biomarkers;
(b) measuring oxidative stress; and
(c) assessing clinical tests of cognitive function.

149. The method of claim 148, wherein the brain specific biomarker is selected from the group consisting of brain derived neurotrophic factors, nerve growth factor, acetylcholine esterase, acetylcholine transferase, Dkk1, gsk-3 beta, fetuin and inflammatory cytokines.
